(19) United States
(12) Patent Application Publication
(10) Pub. No.: US 2008/0081781 A1
(43) Pub. Date: Apr. 3, 2008

(54) Methods and compositions for the treatment of metabolic syndrome

(76) Inventors: Arnold S. Lippa, Ridgewood, NJ (US); Jian-Dong Jiang, Beijing (CN); Jing Wei, Nanjing (CN); Wei-Jia Kong, Beijing (CN); Li-Xun Zhao, Beijing (CN); Dan-Qing Song, Beijing (CN)

Correspondence Address: BLACK LOWE & GRAHAM PLLC, Suite 4800, 701 Fifth Avenue, Seattle, WA, 98104, US

(21) Appl. No.: 11/784,294

(22) Filed: Apr. 6, 2007

Related U.S. Application Data
(63) Continuation of application No. 11/502,837, filed on Aug. 10, 2006.
Application No. 11/502,837 is a continuation-in-part of application No. 11/229,339, filed on Sep. 16, 2005.

(30) Foreign Application Priority Data
Nov. 23, 2004 (CN) 200410095066.X
Sep. 17, 2004 (CN) 200410078150.0

(51) Int. Cl. A61K 31/4355; A61K 31/555; A61K 38/28; A61P 3/00

(52) U.S. Cl. 514/3; 514/185; 514/280

(57) ABSTRACT

Methods and compositions containing a berberine compound or berberine related or proto-berberine or derivative compound are provided for the prevention and treatment of metabolic and cardiovascular disorders including metabolic syndrome, hyperlipidemia, obesity, diabetes, insulin resistance, hyperglycemia, hypertension and elevated cholesterol in mammalian subjects. The methods and compositions of the invention are effective for prevention and treatment of metabolic syndrome, hyperlipidemia, obesity, diabetes, insulin resistance, hyperglycemia, hypertension and elevated cholesterol. Additional compositions and methods are provided which employ a berberine compound or berberine related or derivative compound in combination with a second anti-therapeutic agent to yield more effective treatment tools against metabolic disorders, and/or dual activity therapeutic methods and formulations useful to prevent or reduce hyperlipidemia and/or hyperglycemia and one or more causal or related symptoms or conditions associated with hyperlipidemia and/or hyperglycemia in mammalian subjects.

### RELATED APPLICATIONS

This application claims priority as a continuation-in-part of U.S. patent application Ser. No. 11/229,339, filed Sep. 16, 2005, which claims all priority benefits of Chinese Patent Application No. 200410095066.X, filed Nov. 23, 2004; Chinese Patent Application No. 200410078150.0, filed Sep. 17, 2004, each of which is incorporated herein by reference.

### TECHNICAL FIELD

The present invention relates to methods and compositions for treating metabolic and cardiovascular disorders in mammalian subjects. More specifically, the invention relates to methods and compositions for treating and/or preventing metabolic and cardiovascular disorders such as hyperlipidemia, obesity, diabetes, insulin resistance, glucose intolerance, hyperglycemia, metabolic syndrome and hypertension, as well as conditions or complications associated with these metabolic and cardiovascular disorders in mammals.

### BACKGROUND

Metabolic disorders, particularly glucose and lipid regulatory disorders, are becoming increasingly prevalent as the populations in industrialized nations age and sedentary lifestyles become more common. Such disorders are frequently interrelated and are often predictors or results of each other. For example, diabetes is caused by a combination of insulin resistance and defective secretion of insulin by pancreatic-β cells. Individuals with insulin resistance often have abdominal obesity, dyslipidemia, hypertension, glucose intolerance and a prothrombitic state (Metabolic syndrome). Correspondingly, obese individuals as a whole are at higher risk for acquiring insulin resistance. The breakdown of a metabolic pathway thus can trigger myriad disorders such as hyperlipidemia, obesity, diabetes, insulin resistance, glucose intolerance, hyperglycemia, metabolic syndrome and hypertension which may in turn trigger further metabolic dysfunction resulting in systemic issues and putting individuals at risk for additional complications and premature morbidity.

Glucose and lipid levels are regulated in part by the liver which plays a role in synthesizing, storing, secreting, transforming, and breaking down glucose, proteins and lipids. Disease or traumatic injury can greatly reduce the liver's ability to carry out these normal activities. Thus, most of the clinical manifestations of liver dysfunction stem from cell damage and impairment of the normal liver capacities. Liver dysfunction can result from genetic conditions, inflammatory disorders, toxins such as drugs and alcohol, immunological disorders, vascular disorders or metabolic conditions. Regardless of the cause, liver damage can have a systemic effect on the function of metabolic processes and the regulation of blood glucose and serum lipid levels, exacerbating chronic disease states and leading to increased risks for further disease and morbidity.

Both elevated and reduced levels of blood glucose trigger hormonal responses designed to restore glucose homeostasis. Low blood glucose triggers the release of glucagon from pancreatic a-cells. High blood glucose triggers the release of insulin from pancreatic b-cells. ACTH and growth hormones released from the pituitary, act to increase blood glucose by inhibiting uptake by extrahepatic tissues. Glucocorticoids also act to increase blood glucose levels by inhibiting glucose uptake. Cortisol, the major glucocorticoid released from the adrenal cortex, is secreted in response to the increase in circulating ACTH. The adrenal medullary hormone, epinephrine, stimulates production of glucose by activating glycogenolysis in response to stressful stimuli.

Released glucagon binds to receptors on the surface of liver cells and triggers an increase in cAMP production leading to an increased rate of glycogenolysis by activating glycogen phosphorylase via the PKA-mediated cascade. This is the same response hepatocytes have to epinephrine release. The resultant increased levels of glucose 6 phosphatase in hepatocytes are hydrolyzed to free glucose which then diffuses to the blood. The glucose enters extrahepatic cells where it is re-phosphorylated by hexokinase. Since muscle and brain cells lack glucose-6-phosphatase, the glucose-6-phosphate product of hexokinase is retained and oxidized by these tissues.

In opposition to the cellular responses to glucagon, insulin stimulates extrahepatic uptake of glucose from the blood and inhibits glycogenolysis in extrahepatic cells, stimulating glycogen synthesis. The released insulin binds to the insulin receptor (InsR), an integral cell membrane glycoprotein found on hepatic cells as well as in muscle tissue and lymphocytes. Binding of insulin by InsR triggers an intracellular insulin pathway that includes InsR activation, insulin receptor substrates (IRS) phosphorylation as well as serial downstream activation of phosphoinosital-3-kinase (PI3K), phosphoinositide-dependent kinase (PDK1), protein kinase B (PKB/Akt) and Map Kinase. (Salitel, Cell 104 (4) 517-529 (2001); Kido, J. Clin Endocrinol Metab. 86 (3) 972-979 (2001)). It causes reduction of hepatic glucose output, synthesis of glycogen, increased uptake of glucose from circulation and increased production of insulin in pancreatic beta cells, thus lowering blood glucose. However, once the liver is saturated with glycogen, (roughly 5% of liver mass), further synthesis is strongly suppressed. Additional glucose taken up by hepatocytes is shunted into pathways leading to synthesis of fatty acids, which are exported from the liver as lipoproteins. The lipoproteins are disassembled in the circulation, providing free fatty acids for use in other tissues, including adipocytes, which use them to synthesize triglyceride. Insulin inhibits breakdown of fat in adipose tissue by inhibiting the intracellular lipase that hydrolyzes triglycerides to release fatty acids and facilitates entry of glucose into adipocytes, and within those cells, glucose can be used to synthesize glycerol. Glycerol, along with the fatty acids delivered from the liver, are used to synthesize triglycerides within the adipocyte. By these mechanisms, insulin is involved in further accumulation of triglycerides in fat cells.

There are at least five distinct lipoproteins in mammals, each of which differs in size, composition, density and function. In the cells of the small intestine, dietary lipids are packaged into large lipoprotein complexes called “chylomicrons,” which have a high triglyceride and low cholesterol content. In the liver, triglycerides and cholesterol esters are packaged and released into plasma as triglyceride-rich lipoproteins called very low-density lipoproteins (VLDLs), which primarily transport triglycerides made in the liver or released by adipose tissue. Through enzymatic action, VLDLs can either be reduced and taken up by the liver or transformed into intermediate density lipoproteins (IDLs). IDLs are in turn either taken up by the liver or further modified to form low density lipoproteins (LDLs). LDLs are either taken up and broken down by the liver, or taken up by extrahepatic tissue. High density lipoproteins (HDLs) help remove cholesterol from peripheral tissues in a process called reverse cholesterol transport. Some forms of lipoproteins, such as LDLs, are considered “bad” cholesterol and increase the risk of heart disease or other diseases caused by plaque formation. Other forms, such as HDLs, are considered “good” cholesterol and are essential for good health.

LDL metabolism is regulated by the liver low-density protein receptor (LDLR). Increased LDLR expression results in improved clearance of plasma LDL through receptor-mediated endocytosis, lowering plasma LDL levels and reducing the incidence of arterial plaque formation. LDLR expression is generally regulated at the transcriptional level through a negative feedback mechanism by the intracellular cholesterol pool. This regulation is controlled through interactions of the sterol regulatory element (SRE-1) of the LDLR promoter and SRE binding proteins (SREBPs). In the inactive state, SREBP associates with SREBP-cleavage activating protein (SCAP). SCAP contains a cholesterol-sensing domain, which responds to the depletion of sterol with activation of the SCAP-SREBP transporting activity. Under cholesterol depleted conditions, SCAP transports SREBP to the Golgi apparatus where the N-terminal transcription activation domain for the SREBP is released from the precursor protein through specific cleavages. The active form of the SREBP translocates to the nucleus, binds to its cognate SRE-1 site and activates transcription of the LDLR gene. When there is enough cholesterol, the SCAP-SREBP complex remains in an inactive form in the endoplasmic reticulum through active repression by sterols, and LDLR gene transcription is maintained at a minimal constitutive level.

Insulin and tri-iodothyronine (T3) increase the binding of LDLs to liver cells, whereas glucocorticoids (e.g., dexamethasone) have the opposite effect. The effects of insulin and T3 on hepatic LDL binding may explain the hypercholesterolemia and increased risk of atherosclerosis and other forms of cardiovascular disease that have been shown to be associated with uncontrolled diabetes or hypothyroidism.

Metabolic disorders that effect glucose and lipid metabolism such as hyperlipidemia, obesity, diabetes, insulin resistance, hyperglycemia, glucose intolerance, metabolic syndrome and hypertension have long term health consequences leading to chronic conditions including cardiovascular disease and premature morbidity. Such metabolic and cardiovascular disorders may be interrelated, aggravating or triggering each other and generating feedback mechanisms that are difficult to interrupt.

Current pharmaceutical treatments for metabolic and cardiovascular disorders include combinations of lipid-lowering drugs, hypoglycemic drugs, anti-hypertensive agents, diet and exercise. However, complicated therapeutic regimens can cause polypharmacy problems of increased side effects, drug-drug interactions, failure of adherence, and increased medication errors. (Grundy, Nat Rev, Drug Discov 5 (4), 295-309 (2006)). There is therefore a compelling, unmet need in the art to identify new compounds, formulations and methods to safely and effectively treat metabolic and cardiovascular disorders and conditions associated with metabolic disorders.

### SUMMARY OF THE EXEMPLARY EMBODIMENTS OF THE INVENTION

It is therefore an object of the present invention to provide novel and improved compositions and methods for treating and managing metabolic and cardiovascular disorders in mammalian subjects, including humans.

It is a further object of the present invention to provide novel and improved compositions and methods for treating and managing liver dysfunction in mammalian subjects, including humans.

It is another object of the present invention to provide novel and improved compositions and methods for treating and managing metabolic syndrome in mammalian subjects, including humans.

It is yet another object of the present invention to provide novel and improved compositions and methods for treating and managing hyperlipidemia in mammalian subjects, including humans.

It is another object of the present invention to provide novel and improved compositions and methods for treating and managing hyper-cholesterolemia in mammalian subjects, including humans.

It is still another object of the present invention to provide novel and improved compositions and methods for treating and managing diabetes in mammalian subjects, including humans.

It is a further object of the present invention to provide novel and improved compositions and methods for treating and managing insulin resistance in mammalian subjects, including humans.

It is a further object of the present invention to provide novel and improved compositions and methods for treating and managing hyperglycemia in mammalian subjects, including humans.

It is a yet another object of the present invention to provide novel and improved compositions and methods for treating and managing hypertension in mammalian subjects, including humans.

It is a further object of the present invention to provide novel and improved compositions and methods for increasing insulin sensitivity in mammalian subjects, including humans.

It is a further object of the present invention to provide novel and improved composition and methods for treating obesity in mammalian subjects, including humans.

It is a further object of the invention to provide compositions and methods for treating and preventing diseases triggered or aggravated by metabolic and cardiovascular disorders including, but not limited to, fatty liver, reproductive abnormalities, growth abnormalities, arterial plaque accumulation, osteoarthritis, gout, joint pain, respiratory problems, skin conditions, sleep apnea, idiopathic intracranial hypertension, lower extremity venous stasis disease, gastro-esophageal reflux, urinary stress incontinence, kidney damage, cardiovascular diseases such as atherosclerosis, coronary artery disease, enlarged heart, diabetic cardiomyopathy, angina pectoris, peripheral vascular disease, carotid artery disease, stroke, cerebral arteriosclerosis, myocardial infarction, cerebral infarction, restenosis following balloon angioplasty, intermittent claudication, dyslipidemia post-prandial lipidemia, high blood pressure and xanthoma.

The invention achieves these objects and satisfies additional objects and advantages by providing novel and surprisingly effective methods and compositions for treating and/or preventing metabolic and cardiovascular disorders including, but not limited to, metabolic syndrome, hyperlipidemia, obesity, diabetes, insulin resistance, hyperglycemia, glucose intolerance and hypertension in mammalian subjects employing berberine and related compounds, derivatives, and proto-berberine compounds and derivatives according to formula I, below. 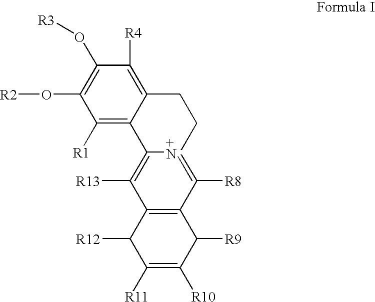
wherein each of R₁, R₂, R₃, R₄, R₈, R₉, R₁₀, R₁₁, R₁₂ and/or R₁₃ may independently, collectively, or in any combination that yields an active (e.g., anti-dyslipidemic, anti-hyperlipidemic, anti-hyperglycemic, anti-hypertensive, LDL-modulatory, LDLR-modulatory, or insulin receptor (InsR) modulatory) compound according to this disclosure, be a hydrogen, halogen, hydroxy, alkyl, alkoxy, nitro, amino, trifluoromethyl, cycloalkyl, (cycloalkyl)alkyl, alkanoyl, alkanoyloxy, aryl, aroyl, aralkyl, nitrile, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, alkanoylamino, carbamoyl, carbamyl, carbonylamino, alkylsulfonylamino, heterocyclo group, or oligosaccharide. When more than one R group is present, the R group may be selected from any of the stated groups so as to be the same or different. In certain exemplary embodiments, the following illustrative structural modifications according to Formula I above will be selected to provide useful candidate compounds for treating and/or preventing metabolic and cardiovascular disorders in mammalian subjects, e.g., wherein: R₁ is selected from methyl, ethyl, hydroxyl, or methoxy; R₂ is selected from H, methyl, ethyl, methene; R₃ is selected from H, methyl, ethyl, methene; R₄ is selected from methyl, ethyl, hydroxyl, or methoxy; R₈ is selected from straight or branched (C1-C6)alkyl (e.g., substitution selected from methyl, ethyl, n-propyl, 1-methylethyl, n-butyl, 1-methylpropyl, 2-methylpropyl, 1,1-dimethylethyl, n-pentyl, 2-methylbutyl, 1,1-dimethylpropyl, 2,2 dimethylpropyl, 3-methylbutyl, n-hexyl, 1-methylpentyl, 1,1-dimethylbutyl, 2,2-dimethylbutyl, 3-methylpentyl, 1,2-dimethylbutyl, 1,3-dimethyl and 1-methyl-2ethylpropyl); R₉ is selected from methyl, ethyl, hydroxyl, Cl, Br; R₁₀ is selected from methyl, ethyl, hydroxyl, Cl, Br; R₁₁ is selected from methyl, ethyl, hydroxyl, Cl, Br; R₁₂ is selected from methyl, ethyl, hydroxyl, Cl, Br; and R₁₃ is selected from straight or branched (C1-C6)alkyl (e.g., substitution selected from methyl, ethyl, n-propyl, 1-methylethyl, n-butyl, 1-methylpropyl, 2-methylpropyl, 1,1-dimethylethyl, n-pentyl, 2-methylbutyl, 1,1-dimethylpropyl, 2,2 dimethylpropyl, 3-methylbutyl, n-hexyl, 1-methylpentyl, 1,1-dimethylbutyl, 2,2-dimethylbutyl, 3-methylpentyl, 1,2-dimethylbutyl, 1,3-dimethyl and 1-methyl-2ethylpropyl). Additional candidate compounds for use within the compositions and methods will be readily produced and selected according to the further disclosure provided herein below.

Useful berberine and related compounds and derivatives and proto-berberine compounds and derivatives within the formulations and methods of the invention include, but are not limited to, salts of berberine and related or derivative compounds, for example, berberine sulfate, berberine hydrochloride, berberine chloride, palmatine chloride, palmatine, oxyberberine, dihydroberberine, 8-cyanodihydroberberine, (−)-canadine, tetrahydroberberine N-oxide, tetrahydroberberine, N-methyltetrahydroberberinium iodide, 6-protoberberine, 9-ethoxycarbonyl berberine, 9-N,N-dimethylcarbamoyl berberine and 12-bromo berberine, berberine azide, and berberine betaine. Other useful forms of berberine and related compounds and derivatives and proto-berberine compounds and derivatives for use within the invention include other pharmaceutically acceptable active salts of said compounds, as well as active isomers, enantiomers, polymorphs, glycosylated derivatives, solvates, hydrates, and/or prodrugs of said compounds.

In exemplary embodiments, the compositions and methods of the invention employ a berberine compound or a berberine related or derivative compound of Formula I to treat and/or prevent symptoms of metabolic and cardiovascular disorders or another disease or condition associated with metabolic disorders, such as a cardiovascular disease.

Mammalian subjects amenable for treatment with berberine, berberine related and derivative compounds, and proto-berberine compounds and derivatives of Formula I according to the methods of the invention include, but are not limited to, subjects with hyperlipidemia and subjects with elevated cholesterol, including subjects presenting with, or at elevated risk for developing, elevated LDL, elevated cholesterol, and/or elevated triglyceride levels; subjects with hyperglycemia; subjects with elevated blood glucose levels; subjects with diabetes; subjects with insulin resistance; subjects with elevated blood pressure; subjects with obesity; subjects with decreased insulin sensitivity; subjects in a prothrombotic state; subjects in a proinflammatory state.

These and other subjects are effectively treated, prophylactically and/or therapeutically, by administering to the subject a metabolic correcting effective amount (or, alternatively, an anti-dyslipidemic, anti-hyperlipidemic, anti-hyperglycemic, anti-hypertensive, LDL-modulatory, LDLR-modulatory, insulin receptor (InsR) modulatory effective amount) of a berberine, berberine related compound or derivative, or proto-berberine compounds and derivatives of Formula I sufficient to prevent or reduce metabolic and cardiovascular disorders including metabolic syndrome, hyperlipidemia, obesity, diabetes, insulin resistance, hyperglycemia, and hypertension or one or more disease symptoms or conditions associated with metabolic and cardiovascular disorders (or, alternatively, to elicit an anti-dyslipidemic, anti-hyperlipidemic, anti-hyperglycemic, anti-hypertensive, anti-metabolic disorder, LDL-modulatory, LDLR-modulatory, or InsR modulatory response) in the subject. The therapeutically useful methods and formulations of the invention will effectively use berberine, berberine related and derivative compounds, and proto-berberine compounds and derivatives of Formula I in a variety of forms, as noted above, including any active, pharmaceutically acceptable salt of said compounds, as well as active isomers, enantiomers, polymorphs, solvates, hydrates, prodrugs, and/or combinations thereof. Berberine is therefore employed as an illustrative embodiment of the invention within the examples herein below.

In additional embodiments of the invention, mammalian subjects are effectively treated, prophylactically and/or therapeutically, by administering to the subject a cholesterol-controlling effective amount of a berberine compound, related or derivative compound of Formula I, or proto-berberine compound and derivative sufficient to prevent or reduce elevated cholesterol, or one or more associated symptoms or condition(s), in the subject. These therapeutically useful methods and formulations of the invention may likewise employ a berberine compound, related or derivative compound of Formula I, or proto-berberine compound or derivative in a variety of forms, including pharmaceutically acceptable salts, isomers, enantiomers, polymorphs, solvates, hydrates, prodrugs, and/or combinations thereof.

In further embodiments of the invention, mammalian subjects are effectively treated, prophylactically and/or therapeutically, by administering to the subject a glucose-controlling effective amount of a berberine compound, a related or derivative compound of Formula I, or a proto-berberine compound or derivative sufficient to prevent or reduce elevated blood glucose, or one or more associated symptoms or condition(s), in the subject. These therapeutically useful methods and formulations of the invention may likewise employ a berberine compound, related or derivative compound of Formula I, or a proto-berberine compound or derivative in a variety of forms, including pharmaceutically acceptable salts, isomers, enantiomers, polymorphs, solvates, hydrates, prodrugs, and/or combinations thereof.

Within additional aspects of the invention, combinatorial formulations and methods are provided which employ an effective amount of a berberine compound (or of another berberine related or derivative compound of formula I, or proto-berberine compound or derivative) in combination with one or more secondary or adjunctive active agent(s) that is/are combinatorially formulated or coordinately administered with the berberine, berberine related or derivative compound, or proto-berberine compound or derivative to yield cholesterol lowering and/or glucose lowering effective response (or, alternatively, an anti-dyslipidemic, anti-hyperlipidemic, anti-hypercholesterolemic, anti-hyperglycemic, anti-metabolic syndrome, insulin sensitivity increasing, insulin resistance decreasing, anti-diabetic, anti-obesity, anti-hypertensive, anti-metabolic disorder, LDL-modulatory, LDLR-modulatory, or InsR modulatory response) in the subject. Exemplary combinatorial formulations and coordinate treatment methods in this context employ the berberine, berberine related or derivative compound of Formula I, or proto-berberine compound or derivative in combination with one or more additional, lipid and/or glucose lowering agent(s) or other indicated, secondary or adjunctive therapeutic agents. The secondary or adjunctive therapeutic agents used in combination with, e.g., berberine in these embodiments may possess direct or indirect lipid and/or glucose lowering activity and/or hypertension decreasing activity, including cholesterol lowering activity, insulin resistance decreasing activity, insulin sensitivity increasing activity or glucose regulating activity, alone or in combination with, e.g., berberine, or may exhibit other useful adjunctive therapeutic activity in combination with, e.g., berberine.

Useful adjunctive therapeutic agents in these combinatorial formulations and coordinate treatment methods include, for example, anti-hyperlipidemic agents; anti-dyslipidemic agents; plasma HDL-raising agents; anti-hypercholesterolemic agents, including, but not limited to, cholesterol-uptake inhibitors; cholesterol biosynthesis inhibitors, e.g., HMG-CoA reductase inhibitors (also referred to as statins, such as lovastatin, simvastatin, pravastatin, fluvastatin, rosuvastatin, pitavastatin, and atorvastatin); HMG-CoA synthase inhibitors; squalene epoxidase inhibitors or squalene synthetase inhibitors (also known as squalene synthase inhibitors); acyl-coenzyme A cholesterol acyltransferase (ACAT) inhibitors, including, but not limited to, melinamide; probucol; nicotinic acid and the salts thereof; niacinamide; cholesterol absorption inhibitors, including, but not limited to, P-sitosterol or ezetimibe; bile acid sequestrant anion exchange resins, including, but not limited to cholestyramine, colestipol, colesevelam or dialkylaminoalkyl derivatives of a cross-linked dextran; LDL receptor inducers; fibrates, including, but not limited to, clofibrate, bezafibrate, fenofibrate and gemfibrozil; vitamin B6 (also known as pyridoxine) and the pharmaceutically acceptable salts thereof, such as the HCl salt; vitamin B12 (also known as cyanocobalamin); vitamin B3 (also known as nicotinic acid and niacinamide, supra); anti-oxidant vitamins, including, but not limited to, vitamin C and E and betacarotene; angiotensin II receptor (AT₁) antagonist, renin inhibitors; platelet aggregation inhibitors, including, but not limited to, fibrinogen receptor antagonists, i.e., glycoprotein IIb/IIIa fibrinogen receptor antagonists; hormones, including but not limited to, estrogen; insulin; ion exchange resins; omega-3 oils; benfluorex; ethyl icosapentate; and amlodipine; appetite-suppressing agents or anti-obesity agents including, but not limited to, insulin sensitizers, protein tyrosine phosphatase-1B (PTP-1B) inhibitors, dipeptidyl peptidase IV (DP-IV) inhibitors, insulin or insulin mimetics, sequestrants, nicotinyl alcohol, nicotinic acid, PPARα agonists, PPAR γ agonists including, but not limited to glitazones, PPARα/γ dual agonists, inhibitors of cholesterol absorption, acyl CoA:cholesterol acyltransferase inhibitors, anti-oxidants, anti-obesity compounds, neuropeptide Y5 inhibitors, β₃ adrenergic receptor agonists, ileal bile acid transporter inhibitors, anti-inflammatories and cyclo-oxygenase 2 selective inhibitors; insulin; sulfonylureas, including but not limited to chlorpropamide, glipizide, glyburide, and glimepiride; DPP-4 blockers; biguanides, including but not limited to metformin; thiazolidinediones including but not limited to rosiglitazone, troglitazone and pioglitazone; alpha-glucosidase inhibitors, including, but not limited to, acarbose and meglitol; cannabinoid antagonists, including, but not limited to rimonabant; camptothecin and camptothecin derivatives, D-phenylalanine derivatives; meglitinides; diuretics including, but not limited to, methyclothiazide, hydroflumethiazide, metolazone, chlorothiazide, methyclothiazide, hydrochlorothiazide, quinethazone, chlorthalidone, trichlormethiazide, bendroflumethiazide, polythiazide, hydroflumethiazide, spironolactone, triamterene, amiloride, bumetanide, torsemide, ethacrynic acid, furosemide; beta-blockers including, but not limited to acebutolol, atenolol, betaxolol, bisoprolol, carteolol, metoprolol, nadolol, pindolol, propranolol, and timolol; angiotensin-converting enzyme (ACE) inhibitors including, but not limited to, benazepril, captopril; enalapril, fosinopril, lisinopril, moexipril, perindopril, quinapril, ramipril, and trandolapril; calcium channel blockers including, but not limited to, amlodipine, diltiazem, felodipine, isradipine, nicardipine sr, nifedipine er, nisoldipine, and verapamil; vasodilators including, but not limited to, nitric oxide, hydralazine, and prostacyclin; angiotensin II receptor blockers including, but not limited to, andesartan, eprosartan, irbesartan, losartan, olmesartan, telmisartan, and valsartan; alpha blockers including, but not limited to, doxazosin, prazosin and terazosin; alpha 2 agonists including, but not limited to clonidine and guanfacine. Such agents may be referred to in whole or in part as metabolic disorder therapeutics, metabolic syndrome therapeutics, anti-obesity therapeutics, anti-hypercholesterolemia therapeutics, anti-diabetic therapeutics, insulin resistance therapeutic agents, anti-hyperglycemia agents, anti-hyperlipidemia agents, insulin sensitivity increasing agents, anti-hypertensive agents, and/or blood glucose lowering therapeutic agents. Adjunctive therapies may also be used including, but not limited, physical treatments such as changes in diet, psychological counseling, behavior modification, exercise and surgery including, but not limited to, gastric partitioning procedures, jejunoileal bypass, stomach stapling, gastric bands, vertical banded gastroplasty, laparoscopic gastric banding, roux-en-Y gastric bypass, biliopancreatic bypass procedures and vagotomy. Some herbal remedies may also be employed effectively in combinatorial formulations and coordinate therapies for treating metabolic disorders, for example curcumin, gugulipid, garlic, vitamin E, soy, soluble fiber, fish oil, green tea, carnitine, chromium, coenzyme Q10, anti-oxidant vitamins, grape seed extract, pantothine, red yeast rice, and royal jelly.

The forgoing objects and additional objects, features, aspects and advantages of the instant invention will become apparent from the following detailed description.

### BRIEF DESCRIPTION OF THE DRAWINGS

FIG. 1 is a drawing of the promoter region of the LDL receptor gene. Three direct repeats and two TATA-like sequences are identified with the promoter region. The cis-acting element of sterols is located on repeat 2, whereas the regulatory element for cytokine OM (SIRE) overlaps the TATA-like sequences.

FIG. 2 is a schematic representation of intracellular regulation of LDL receptor gene expression, including regulation by berberine.

FIGS. 3 A and B are quantitative RT-PCR of LDLR mRNA levels in human liver BEL-7402 cells twenty-four hours after being treated with berberine (A) or berberine sulfate (B).

FIG. 4 is a measurement using flow cytometry of the concentration of the protein level of LDLR expressed on the cell surface of BEL-7402 cells twenty-four hours after treatment with 15 μg/ml of berberine.

FIGS. 5 A-C are charts of the decrease in serum cholesterol (A) and LDL (B) in hamsters after treatment with berberine and the decrease of LDL as a function of time (C).

FIG. 6 is a depiction of the concentration of total LDLR mRNA and protein extracts as measured by quantitative real time RT-PCR (A) and Western blot (B) in hamsters sacrificed four hours after the last treatment with berberine.

FIG. 7 is a Western Blot showing the concentration of the precursor (P) and mature (M) forms of SREBP2 using a monoclonal antibody to SREBP2 in HepG2 cells.

FIG. 8 is (A) a northern blot showing LDLR expression in HepG2 cells treated with either lovastatin (Lov) alone or in combination with berberine (BBR) for 24 hours and (B) a chart of real-time RT-PCR of the same cells.

FIG. 9 is a chart showing the increase in LDLR promoter activity in the presence of GW707 and oncostatin M.

FIG. 10 is (A) a northern blot showing concentrations of LDLR mRNA in HepG2 cells treated with berberine in the presence of different concentrations of actinomycin D and (B) a plot of normalized LDLR mRNA signals as a percentage of LDLR mRNA remaining.

FIG. 11 is a schematic representation of the LDLR mRNA 3′ UTR and the chimeric Luc-LDLR 3′ UTR constructs.

FIG. 12 is a northern blot of analysis of Luc-LDLR fusion mRNA in (A) control cells and cells treated with (B) berberine or dimethylsulfoxide as a control.

FIG. 13 is a schematic representation of the constructs containing the deletions of ARE and UCAU motifs (B) and a chart illustrating the responses of the wt pLuc/UTR-2 and deletion constructs to berberine treatment as determined by real-time RT-PCR analysis.

FIG. 14 is a western blot of cellular proteins harvested from (A) Bel-7402 cells or (B) HepG2 cells that were untreated or treated with berberine at a dose of 5 μg/ml for different levels as indicated and (C) a western blot of HepG2 cells treated for 1 hour at the indicated concentrations.

FIG. 15 (A) is a chart depicting a dose dependent increase in the expression of InsR mRNA in human hepatoma cells treated with berberine as measured using real time PCR and (B) confirmed by slot blot.

FIG. 16 (A) is a chart depicting the time-dependent effect of berberine on InsR mRNA expression in human hepatoma cells over 24 hours as confirmed by (B) slot blot.

FIG. 17 (A-F) are graphs depicting increased cell surface InsR expression in Caucasian liver cell line HepG2 when treated with (A) IgG, (B) 0 μg/ml of berberine, (C) 2.5 μg/ml of berberine, (D) 5 μg/ml of berberine, (E) 10 μg/ml of berberine, and (F) 15 μg/ml of berberine.

FIG. 18 (A-F) are graphs depicting increased cell surface InsR expression in Asian liver cell line Bel-7402 when treated with (A) IgG, (B) 0 μg/ml of berberine, (C) 2.5 μg/ml of berberine, (D) 5 μg/ml of berberine, (E) 10 μg/ml of berberine, and (F) 15 μg/ml of berberine.

FIG. 19 (A-B) are charts showing that (A) berberine increases glucose consumption in the presence of InsR expression and insulin and that (B) silencing InsR expression abolishes the glucose consumption effect.

FIG. 20 (A-D) are charts showing that treatment of human liver cells with 7.5 μg/ml of berberine increases the expression of both InsR and LDLR.

FIG. 21 (A-B) are (A) a slot blot of the amount of InsR mRNA in HepG2 cells untreated (column C) or treated with berberine (column BBR) and then treated with actinomycin and normalized with ACTB and (B) a chart of the data plotted as a the percentage of the InsR mRNA remaining.

FIG. 22 is a chart showing the dose dependent increase of Luc mRNA in pGl3-1.5kIRP transfected cells incubated with berberine (BBR) for eight hours.

FIG. 23 is a chart showing RT-PCR measurements of the amount of InsR and LDLR mRNA in HepG2 cells treated with calphostin (Cal), berberine (BBR) or a combination of calphostin and berberine.

FIG. 24 is a chart showing the relative amounts of InsR and LDLR mRNA as measured by RT-PCR in HepG2 cells treated with U0126, berberine or a combination of U0126 and berberine.

FIG. 25 (A) is a picture of a gel of phosphorylated and nonphosphorylated substrates in cell lysates of HepG2 cells treated with berberine for 0, 0.25, 1, 2, and 4 hours and (B) a chart of the quantification of protein kinase C (PKC) activity using densitometry and expressed as the number of picomoles of phosphate transferred to the substrate per minute per milligram of sample protein.

FIG. 26 is a chart showing luciferase activity representing normalized InsR promoter activity in pGL3-1.5kIRP transfected HepG2 cells treated with calphostin, berberine, phorbol 12-myristate 13-acetate (PMA) or combinations as shown.

FIG. 27 is a graph of the decline in the fasting blood glucose of hyperglycemic rats treated with berberine.

FIG. 28 is a chart of liver InsR and LDLR mRNA of rats treated with berberine as calculated by RT-PCR.

FIG. 29 is a chart of dose-dependent induction of InsR mRNA expression in HepG2 cells incubated with berberine for eight hours as measured by RT-PCR with the amount of InsR mRNA in untreated cells defined as “1” and the amounts of InsR mRNA from berberine treated cells plotted relative to that value.

FIG. 30 (A) is a picture of a gel showing the phosphorylated and nonphosphorylated substrates in cell lysates of liver samples of rats treated with berberine and (B) a chart of the quantification of PKC activity using densitometry and expressed as the number of picomoles of phosphate transferred to the substrate per minute per milligram of sample protein.

FIG. 31 is a chart showing the decrease in the level of fasting serum insulin in hyperglycemic rats on a high fat and high cholesterol (HFHC) diet when they were treated with berberine.

FIG. 32 is a chart of the increase in insulin sensitivity index (ISI) in hyperglycemic rats on a four week HFHC diet when treated with berberine.

FIG. 33 is a chart of the decrease in serum lipid levels in hyperlipidemic rats treated with berberine.

FIG. 34 is a schematic of InsR and LDLR expression and their upregulation by berberine.

FIG. 35 is a chart of serum insulin levels in hyperglycemic patients as measured before and after two months of therapy with berberine.

FIG. 36 is a chart of InsR expression on the surface of peripheral blood lymphocytes (PBL) of hyperglycemic patients as measured before and after two months of therapy with berberine.

FIG. 37 (A-H) are charts showing the negative correlation between InsR expression on the surface of peripheral blood lymphocytes and fasting blood glucose levels in eight patients 0, 15 and 60 days after treatment with berberine.

FIG. 38 is a diagram of cholesterol synthesis by the body.

### DETAILED DESCRIPTION OF EXEMPLARY EMBODIMENTS OF THE INVENTION

The instant invention provides novel methods and compositions for preventing and/or treating metabolic and cardiovascular disorders including but not limited to metabolic syndrome, hyperlipidemia, hypercholesterolemia, obesity, diabetes, insulin resistance, hyperglycemia, hypertension and elevated cholesterol in mammalian subjects, including individuals and in vitro, ex vivo, and in vivo mammalian cells, tissues, and organs. In various embodiments, the methods and compositions are effective to prevent or treat diseases caused by metabolic and cardiovascular disorders including cardiovascular disease.

As used herein, the term “cardiovascular disease” is intended to include a range of symptoms, conditions, and/or diseases including atherosclerosis, coronary artery disease, pulmonary embolism, diabetic cardiomyopathy, angina pectoris, carotid artery disease, strokes, peripheral vascular disease, cerebral arteriosclerosis, myocardial infarction, high blood pressure, cerebral infarction, restenosis following balloon angioplasty, intermittent claudication, dyslipidemia post-prandial lipidemia and xanthoma, and all conventionally targeted symptoms arising from or associated with the foregoing diseases and conditions.

Anti-metabolic disorder formulations and methods provided herein employ a berberine compound, berberine related or derivative compound of Formula I, above, or proto-berberine compound or derivative, including glycosylated derivatives, all active pharmaceutically acceptable compounds of this description as well as various foreseen and readily provided complexes, salts, solcates, isomers, enantiomers, polymorphs, and prodrugs of these compounds and combinations thereof as novel glucose or lipid lowering agents. Exemplary compounds for use within the invention include, as illustrative embodiments, berberine sulfate, berberine chloride, (−)-canadine, berberine hydrochloride, palmatine chloride, palmatine, oxyberberine, dihydroberberine, 8-cyanodihydroberberine, tetrahydroberberine N-oxide, tetrahydroberberine, N-methyltetrahydroberberinium iodide, 6-protoberberine, 9-ethoxycarbonyl berberine, 9-N,N-dimethylcarbamoyl berberine and 12-bromo berberine, berberine azide, and berberine betaine.

Lipid lowering formulations and methods provided herein, including cholesterol lowering formulations and methods, employ a berberine compound, berberine related or derivative compound of Formula I, above, or proto-berberine compound, including all active pharmaceutically acceptable compounds of this description as well as various foreseen and readily provided complexes, derivatives including glycosylated derivatives, salts, solvates, isomers, enantiomers, polymorphs, and prodrugs of these compounds, and combinations thereof, as novel lipid lowering agents. Exemplary compounds for use within the invention include, as illustrative embodiments, berberine sulfate, berberine chloride, (−)-canadine, berberine hydrochloride, palmatine chloride, palmatine, oxyberberine, dihydroberberine, 8-cyanodihydroberberine, tetrahydroberberine N-oxide, tetrahydroberberine, N-methyltetrahydroberberinium iodide, 6-protoberberine, 9-ethoxycarbonyl berberine, 9-N,N-dimethylcarbamoyl berberine and 12-bromo berberine, berberine azide, and berberine betaine.

Glucose lowering formulations and methods provided herein employ a berberine compound, berberine related or derivative compound of Formula I, above, or proto-berberine compound including glycosylated derivatives, all active pharmaceutically acceptable compounds of this description as well as various foreseen and readily provided complexes, salts, solcates, isomers, enantiomers, polymorphs, and prodrugs of these compounds and combinations thereof as novel glucose lowering agents. Exemplary compounds for use within the invention include, as illustrative embodiments, berberine sulfate, berberine chloride, (−)-canadine, berberine hydrochloride, palmatine chloride, palmatine, oxyberberine, dihydroberberine, 8-cyanodihydroberberine, tetrahydroberberine N-oxide, tetrahydroberberine, N-methyltetrahydroberberinium iodide, 6-protoberberine, 9-ethoxycarbonyl berberine, 9-N,N-dimethylcarbamoyl berberine and 12-bromo berberine, berberine azide, and berberine betaine.

Insulin sensitivity increasing formulations and methods provided herein employ a berberine compound, berberine related or derivative compound of Formula I, above, or proto-berberine compound including glycosylated derivatives, all active pharmaceutically acceptable compounds of this description as well as various foreseen and readily provided complexes, salts, solcates, isomers, enantiomers, polymorphs, and prodrugs of these compounds and combinations thereof as novel insulin sensitivity increasing agents. Exemplary compounds for use within the invention include, as illustrative embodiments, berberine sulfate, berberine chloride, berberine hydrochloride, (−)-canadine, palmatine chloride, palmatine, oxyberberine, dihydroberberine, 8-cyanodihydroberberine, tetrahydroberberine N-oxide, tetrahydroberberine, N-methyltetrahydroberberinium iodide, 6-protoberberine, 9-ethoxycarbonyl berberine, 9-N,N-dimethylcarbamoyl berberine and 12-bromo berberine, berberine azide, and berberine betaine.

Insulin resistance decreasing formulations and methods provided herein employ a berberine compound, berberine related or derivative compound of Formula I, above, or proto-berberine compound, including glycosylated derivatives, all active pharmaceutically acceptable compounds of this description as well as various foreseen and readily provided complexes, salts, solcates, isomers, enantiomers, polymorphs, and prodrugs of these compounds and combinations thereof as novel insulin resistance decreasing agents. Exemplary compounds for use within the invention include, as illustrative embodiments, berberine sulfate, berberine chloride, berberine hydrochloride, palmatine chloride, palmatine, (−)-canadine, oxyberberine, dihydroberberine, 8-cyanodihydroberberine, tetrahydroberberine N-oxide, tetrahydroberberine, N-methyltetrahydroberberinium iodide, 6-protoberberine, 9-ethoxycarbonyl berberine, 9-N,N-dimethylcarbamoyl berberine and 12-bromo berberine, berberine azide, and berberine betaine.

Anti-obesity formulations and methods provided herein employ a berberine compound, berberine related or derivative compound of Formula I, above, or proto-berberine compound or derivative, including glycosylated derivatives, all active pharmaceutically acceptable compounds of this description as well as various foreseen and readily provided complexes, salts, solcates, isomers, enantiomers, polymorphs, and prodrugs of these compounds and combinations thereof as novel anti-obesity agents. Exemplary compounds for use within the invention include, as illustrative embodiments, berberine sulfate, berberine chloride, berberine hydrochloride, palmatine chloride, (−)-canadine, palmatine, oxyberberine, dihydroberberine, 8-cyanodihydroberberine, tetrahydroberberine N-oxide, tetrahydroberberine, N-methyltetrahydroberberinium iodide, 6-protoberberine, 9-ethoxycarbonyl berberine, 9-N,N-dimethylcarbamoyl berberine and 12-bromo berberine, berberine azide, and berberine betaine.

Anti-hypertensive formulations and methods provided herein employ a berberine compound, berberine related or derivative compound of Formula I, above, or proto-berberine compound, including glycosylated derivatives, all active pharmaceutically acceptable compounds of this description as well as various foreseen and readily provided complexes, salts, solcates, isomers, enantiomers, polymorphs, and prodrugs of these compounds and combinations thereof as novel hypertension lowering agents. Exemplary compounds for use within the invention include, as illustrative embodiments, berberine sulfate, berberine chloride, berberine hydrochloride, palmatine chloride, palmatine, (−)-canadine, oxyberberine, dihydroberberine, 8-cyanodihydroberberine, tetrahydroberberine N-oxide, tetrahydroberberine, N-methyltetrahydroberberinium iodide, 6-protoberberine, 9-ethoxycarbonyl berberine, 9-N,N-dimethylcarbamoyl berberine and 12-bromo berberine, berberine azide, and berberine betaine.

Metabolic syndrome treating formulations and methods provided herein employ a berberine compound, berberine related or derivative compound of Formula I, above, or proto-berberine compound or derivative, including glycosylated derivatives, all active pharmaceutically acceptable compounds of this description as well as various foreseen and readily provided complexes, salts, solcates, isomers, enantiomers, polymorphs, and prodrugs of these compounds and combinations thereof as novel metabolic syndrome treating agents. Exemplary compounds for use within the invention include, as illustrative embodiments, berberine sulfate, berberine chloride, berberine hydrochloride, (−)-canadine, palmatine chloride, palmatine, oxyberberine, dihydroberberine, 8-cyanodihydroberberine, tetrahydroberberine N-oxide, tetrahydroberberine, N-methyltetrahydroberberinium iodide, 6-protoberberine, 9-ethoxycarbonyl berberine, 9-N,N-dimethylcarbamoyl berberine and 12-bromo berberine, berberine azide, and berberine betaine.

Within the formulations and methods, a berberine compound, berberine related or derivative compound, or proto-berberine compound as disclosed herein is effectively used to treat metabolic and cardiovascular disorders in mammalian subjects suffering metabolic and cardiovascular disorders and conditions associated with metabolic and cardiovascular disorders including but not limited to, fatty liver, reproductive abnormalities, growth abnormalities, arterial plaque accumulation, osteoarthritis, gout, joint pain, respiratory problems, skin conditions, sleep apnea, idiopathic intracranial hypertension, lower extremity venous stasis disease, gastro-esophageal reflux, urinary stress incontinence, kidney damage, cardiovascular diseases such as atherosclerosis, coronary artery disease, peripheral vascular disease, enlarged heart, diabetic cardiomyopathy, pulmonary embolism, angina pectoris, carotid artery disease, stroke, cerebral arteriosclerosis, myocardial infarction, cerebral infarction, restenosis following balloon angioplasty, intermittent claudication, dyslipidemia post-prandial lipidemia, high blood pressure and xanthoma.

A broad range of mammalian subjects, including human subjects, are amenable to treatment using the formulations and methods of the invention. These subjects include, but are not limited to, human and other mammalian subjects presenting with metabolic and cardiovascular disorders or diseases aggravated or triggered by metabolic and cardiovascular disorders such as fatty liver, reproductive abnormalities, growth abnormalities, arterial plaque accumulation, osteoarthritis, gout, joint pain, respiratory problems, skin conditions, sleep apnea, idiopathic intracranial hypertension, lower extremity venous stasis disease, gastro-esophageal reflux, urinary stress incontinence, kidney damage, cardiovascular diseases such as atherosclerosis, coronary artery disease, enlarged heart, peripheral vascular disease, diabetic cardiomyopathy, pulmonary embolism, angina pectoris, carotid artery disease, stroke, cerebral arteriosclerosis, myocardial infarction, cerebral infarction, restenosis following balloon angioplasty, intermittent claudication, dyslipidemia post-prandial lipidemia, high blood pressure and xanthoma.

Within the methods and compositions of the invention, one or more berberine compound(s), berberine related or derivative compound(s), or proto-berberine or derivative compound(s) as disclosed herein is/are effectively formulated or administered as an anti-hyperlipidemia or cholesterol lowering agent effective for treating hyperlipidemia and/or related disorders. In exemplary embodiments, berberine chloride is demonstrated for illustrative purposes to be an anti-hyperlipidemia effective agent in pharmaceutical formulations and therapeutic methods, alone or in combination with one or more adjunctive therapeutic agent(s). The present disclosure further provides additional, pharmaceutically acceptable berberine compounds and berberine related and derivative compounds in the form of a native or synthetic compound, including complexes, derivatives, including glycosylated derivatives, salts, solvates, isomers, enantiomers, polymorphs, and prodrugs of the compounds disclosed herein, and combinations thereof, which are effective as lipid lowering therapeutic agents within the methods and compositions of the invention.

In healthy adults, a relatively constant level of cholesterol in the body (150-200 mg/dL) is maintained primarily by controlling the level of de novo synthesis which is regulated in part by the dietary intake of cholesterol. Slightly less than half of the cholesterol in the body is synthesized de novo with about 20-25% of total daily production occurring in the liver. Other sites of synthesis include the intestines, adrenal glands and reproductive organs. Cholesterol synthesis occurs in the cytoplasm and microsomes through the conversion of acetyl CoA.

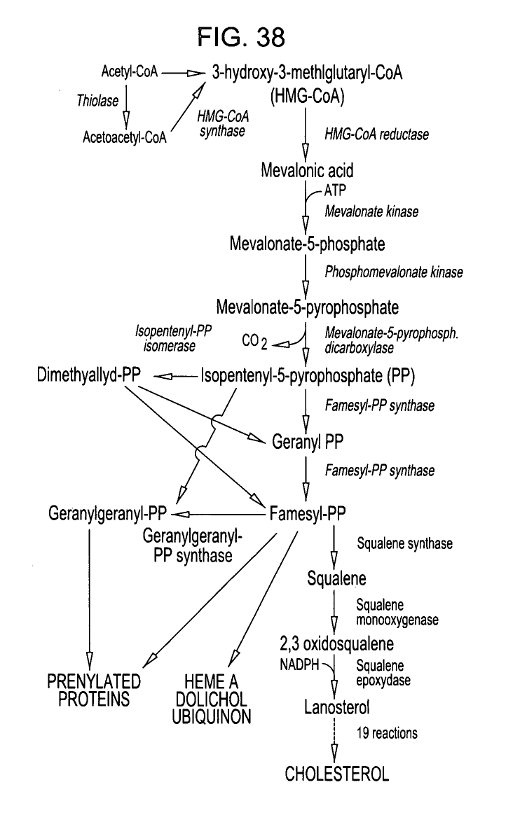

As diagramed in FIG. 38, in order to produce cholesterol, the body converts acetyl-CoA to 3-hydroxy-3-methylglutaryl-CoA (HMG-CoA) which is then converted to mevalonate. Mevalonate is converted to isopentenyl pyrophosphate (IPP) which is converted to squalene. Squalene is then converted to cholesterol. The acetyl-CoA utilized for cholesterol biosynthesis is derived from either an oxidation reaction (e.g., fatty acids or pyruvate) in the mitochondria and is transported to the cytoplasm, or derived from cytoplasmic oxidation of ethanol by acetyl-CoA synthetase.

Normal healthy adults synthesize cholesterol at a rate of approximately 1g/day and consume approximately 0.3 g/day. Cholesterol levels are also maintained through regulation of HMGR activity and levels, regulation of excess intracellular free cholesterol through the activity of acyl-CoA:cholesterol acyltransferase, ACAT and regulation of plasma cholesterol levels via LDL receptor-mediated uptake and HDL-mediated reverse transport.

Hyperlipidemia is an abnormal increase in serum lipids in the bloodstream. It is generally classified as primary hyperlipidemia, which is caused by genetic defects; or secondary hyperlipidemia, which is caused by various disease states, drugs and/or dietary factors. Hyperlipidemia may also result from a combination of primary and secondary causes of hyperlipidemia. Elevated lipoprotein levels, regardless of cause, are associated with a number of disease states, including atherosclerosis, coronary artery disease, angina pectoris, carotid artery disease, stroke, cerebral arteriosclerosis, myocardial infarction, cerebral infarction, restenosis following balloon angioplasty, high blood pressure, intermittent claudication, dyslipidemia, post-prandial lipidemia and xanthoma.

Elevated lipoprotein and glucose levels are frequently found in obese individuals. Obesity is defined as having a body weight that is 20 to 25 percent over the recommended body weight, taking into account a person's particular age, height, and sex. Obesity is a well-established risk factor for a number of potentially life-threatening diseases such as coronary heart disease, osteoarthritis, gout, atherosclerosis, joint pain, sexual and fertility problems, respiratory problems, skin conditions, hypertension, diabetes, stroke, pulmonary embolism, sleep apnea, idiopathic intracranial hypertension, lower extremity venous stasis disease, gastro-esophageal reflux, urinary stress incontinence, and cancer. It also complicates numerous chronic conditions such as respiratory disease, osteoarthritis, osteoporosis, gall bladder disease, and dyslipidemias. The compositions and methods of the present invention are effective in the treatment of all types of hyperlipidemia, regardless of cause.

One cause of hyperlipidemia is liver dysfunction. In normal humans, when dietary cholesterol is increased, de novo synthesis of cholesterol decreases. However in cases of liver dysfunction, this mechanism fails and cholesterol synthesis continues, increasing cholesterol levels in the body and leading to hyperlipidemia. Liver dysfunction can result from genetic conditions, inflammatory disorders, toxins such as drugs and alcohol, immunological disorders, vascular disorders or metabolic conditions. Regardless of the cause, liver damage can have a systemic effect on the function of metabolic processes and the regulation of blood glucose and serum lipid levels, exacerbating chronic disease states and leading to increased risks for further disease and morbidity.

Certain types of diets also interfere with hepatic control of cholesterol synthesis. For example, an increase in the consumption of saturated fats leads to increased levels of plasma cholesterol, particularly increased LDL and VLDL levels. While not wishing to be bound, current theory suggests that saturated triglycerides suppress hepatic LDL receptors leading to elevated LDL levels in plasma. (Ohtani et al. J. of Lipid Res 31 (8): 1413. (1990))

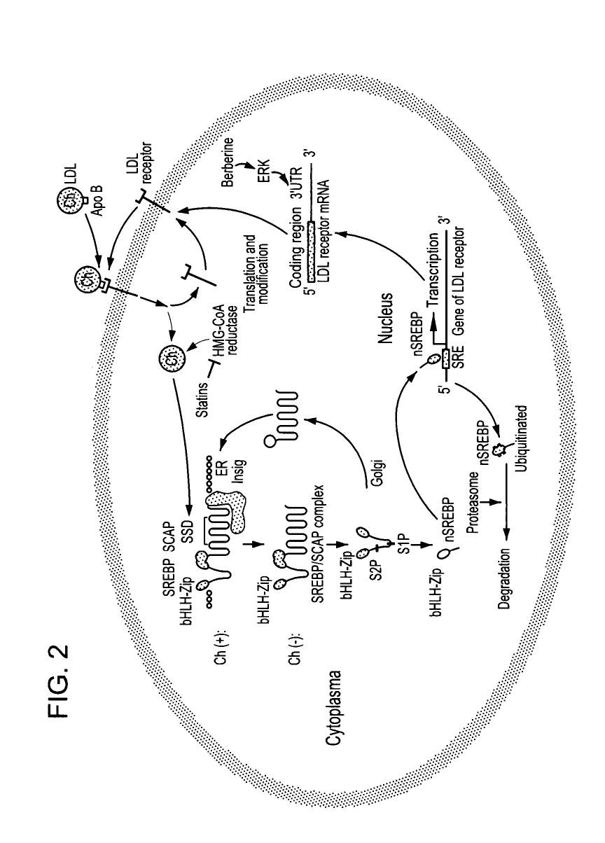

LDL concentrations in plasma are regulated in part by the LDL receptor which captures LDL particles from the bloodstream and draws them inside the cell, clearing them from the bloodstream when there is too much and releasing them when more LDL is needed. Transcriptional regulation of the LDL receptor gene is controlled through the sterol regulatory element-binding protein pathway (SREBP). Bile acid sequestrants, cholesterol biosynthesis inhibitors, and cholesterol absorption inhibitors all influence the SREBP pathway and subsequently upregulate LDL receptor expression. The statins competitively inhibit 3-hydroxy-3-methyl-glutaryl-CoA reductase (HMG-CoA reductase) and block cholesterol biosynthesis in the liver. Hormones, cytokines, growth factors and second messengers also regulate transcription of the LDL receptor gene as outlined in Table 1, below. Post-transcriptional control of the LDL receptor gene is also a target for pharmaceutical intervention. It has been determined in the present invention that berberine is capable of upregulating LDL receptor expression through a post-transcriptional and sterol independent mechanism in hepatocytes (FIG. 2).

TABLE 1
Upregulation of LDL receptor gene expression by different agents

| | | Sterol- | Cis-acting | Trans-acting | Signaling |
|---|---|---|---|---|---|
| Agent(s) | Sites of action | dependence | element(s) | Factors | pathway |
| Statins | Transcription | Dependent | SRE | SREBPs (activated | — |
| | | | | by proteolytic | |
| | | | | cleavage) | |
| Estrogens | Transcription | Independent | Repeat 3 | Estrogen receptor-α | — |
| | | | | and Sp1 | |
| Insulin/ | Transcription | Independent | SRE/SRE + | SREBPs (activated | ERK* |
| growth factors | | | repeat 1 and 3 | by | |
| | | | | phosphorylation)/SR | |
| | | | | EBPs + Sp1 | |
| TNF-α/ | Transcription | Dependent | Unidentified | Unidentified | ERK |
| IL-1] | | | | | |
| OM | Transcription | Independent | SIRE | Egr1 and c/EBP β | ERK |
| PMA | Transcription/post- | Independent | Unidentified/ | Unidentified | PKC |
| | transcription | | 3′ sequence of | | |
| | | | LDL receptor | | |
| | | | 3′ UTR | | |
| Berberine | Post-transcription | Independent | 5′ sequence of | Unidentified | ERK |
| | | | LDL receptor | | |
| | | | 3′ UTR | | |

*c/EBP: CCAAT/enhancer binding protein; Egr1: early growth response gene 1; ERK:

extracellular signal-regulated kinase; IL: interleukin; LDL: low density lipoprotein; OM: oncostatin M; PKC: protein kinase C; PMA: phorbol-12-myristate-13-acetate; SIRE: sterol-independent regulatory element; SRE: sterol regulatory element; SREBP: sterol regulatory element-binding protein; TNF: tumor necrosis factor; UTR: untranslated region.

Those skilled in the art will appreciate that each of the forgoing agents identified in Table 1 that possess activity for regulating LDL receptor expression are useful in combination with the berberine compounds, berberine related and derivative compounds, and proto-berberine compounds described herein, within various combinatorial formulations and coordinate administration methods as described in detail below.

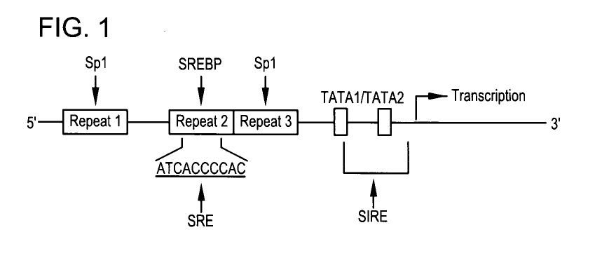

The human LDL receptor structural gene is located in the short arm of chromosome 19. It spans approximately 45 kilobases (kb) and consists of 18 exons, each coding for a different protein domain and 17 introns. (Lindgren et al., PNAS 82:8567-8571 (1985)). The promoter is located on the 5′-flanking region, within which the majority of cis-acting DNA elements are found between base pair (bp)-58 and -234, with the A of the initiator methionine codon as +1. The promoter region spans 177 bp, including three imperfect direct repeats with 16 bp of each, two TATA-like sequences, and several transcription initiation sites, all of which are essential for gene expression and regulation (FIG. 1) (Südhoff et al., Science 228:815-822 (1987)). Repeat 2 contains the 10 bp DNA sterol regulatory element (SRE) (FIG. 1, Smith et al., J. Biol. Chem. 265:2306-2310 (1990)) which controls transcription of the LDL regulator. The human LDL receptor mRNA has a 5.3 kb sequence in length, which contains an unusually 2.5 kb long 3′ untranslated region (UTR) (Yamamoto, Cell 39:27-38 (1984)). There are three AU rich elements (AREs) in the 5′ proximal region and three copies of Alu-like repeat in the 3′ distal region of the 3′UTR. These structures play a key role in the stability of the LDL receptor mRNA which has a constitutively short half life of about 45 minutes in HepG2 cells, and serve as cis-acting elements for the post-transcriptional regulation of the LDL receptor gene expression (Yamamoto et al., Cell 39:27-38 (1984) and Wilson et al., J. Lipid Res. 39:1025-1032 (1998)).

The sterol regulatory element-binding proteins (SREBP) are transcription factors belonging to the basic-helix-loop-helix-leucine zipper (bHLH-Zip) family (Yokoyama et al., Cell 75:187-197 (1993)). They bind to sterol regulatory element (SRE), which is not only present in the promoter of the LDL receptor gene but also in promoters of other genes that code for enzymes participating in cholesterol or fatty acid biosynthesis, such as the HMG-CoA reductase gene and the acetyl coenzyme A synthetase gene (Rawson et al., Mol. Cell. Biol. 4:631-640 (2003)). The major activator of the LDL receptor gene is SREBP-2 (Horton, et al., J. Clin. Invest. 109:1125-1131 (2002).

When cholesterol or its derivatives are abundant in cells, the SREBP pathway is suppressed and the transcription of the LDL receptor gene or other genes required for lipid synthesis are turned off. Abundant cholesterol binds directly to the sterol sensing domain (SSD) of the SREB cleavage-activating protein (SCAP) causing a conformational change which permits SCAP to bind to a pair of endoplasmic reticulum membrane proteins named insulin-induced genes (Insig) 1 and 2, then forms SREBP/SCAP/Insig ternary complex (FIG. 2) (Yang et al., Cell 110:489-500 (2002)). This traps SREBP/SCAP in the endoplasmic reticulum membrane so that the SREBPs are not able to get to the Golgi apparatus for cleavage and the expression levels of LDLR decreases accordingly. As a result, the uptake and synthesis of cholesterol are inhibited, and the cells reach a cholesterol homeostasis (Yang et al., Cell 110:4489-500 (2002)).

When sterols are absent, SCAP does not interact with the Insig proteins. Instead, the SREBP/SCAP complex is free to leave the endoplasmic reticulum and enter the Golgi apparatus (Espenshade et al., PNAS 99:11694-11699 (2002). After arriving in the Golgi apparatus, the transcriptional active domain of the SREBP precursor is released by two sequential proteolytic cleavage catalyzed by two proteases residing in the Golgi membrane, while SCAP returns to the endoplasmic reticulum (FIG. 2) (Brown et al., PNAS 96:11041-11048 (1999) and Nohturfft et al., PNAS 96:11235-11240 (1999). The cleavage of the SREBP precursor results in the release of a fragment containing the bHLH-Zip domain; termed nuclear SREBP (nSREBP), or the mature form of SREBP. The nSREBP enters into the nucleus and activates the transcription LDLR (Brown et al., PNAS 96:11041-11048 (1999). As a result, the cells uptake more cholesterol-containing lipoproteins and increase cholesterol production to reach a new level of cholesterol homeostasis. The nSREBP is not stable, and is polyubiquitinated and rapidly degraded by the proteasome with an estimated half-life of 3 hours (Hirano, et al., J. Biol. Chem. 276:36431-36437 (2001)).

LDL receptor expression can be regulated by such factors as hormones, including estrogen which as an atheroprotective effect and triiodothyronine; insulin and several cytokines including tumor necrosis factor (TNF) α, Interleukin (IL) 1, IL-6 and oncostatin M (OM) all of which activate the transcription of the LDL receptor gene in hepatocytes (Stopeck et al, J. Biol. Chem. 268:17489-17494 (1993)) (Table 1). TNF-α and IL-1 are capable of regulating the LDL receptor gene transcription only when cells are cultured in sterol-free media, and their induction is repressed after sterols or LDL is added (Stopeck et al., J. Biol. Chem. 268:17489-17494 (1993)). OM or IL-6 upregulate the LDL receptor gene expression in a sterol-independent manner, similar to that of insulin and some growth factors (Gierens et al., Arterioscler. Thromb. Vasc. Biol. 20:1777-1783 (2000)). OM has also been shown to increase the LDL receptor gene transcription by recruiting transcription factors early growth response gene 1 (Egr1) and CCAAT/enhancer binding protein β (c/EBP β) to bind to a DNA motif termed sterol-independent regulatory element (SIRE) which overlaps the TATA-like sequences in the promoter region of the LDL receptor gene (FIG. 1), whereas IL-6 needs SRE and the repeat 3 Sp1 binding site for mediating its transcriptional activation effect factors (Gierens et al., Arterioscler. Thromb. Vasc. Biol. 20:1777-1783 (2000)).

Growth factors including the platelet-derived growth factor (PDGF), EGF and the fibroblast growth factor (FGF) also upregulate LDL receptor gene expression (Basheeruddin et al., Arterioscler. Thromb. Vasc. Biol. 15:1248-1254 (1995)). The stimulation effect of growth factors on the LDL receptor gene promoter requires SRE as well as the Sp1 binding sites as cis-acting elements, and is related to the ERK mediated phosphorylation and activation of SREBPs, as growth factors potently activate this signaling pathway just like insulin (Kotzka et al., J. Lipid. Res. 41:99-108 (2000)). Second messenger analog phorbol esters regulate the LDL receptor gene expression as well.

The above-mentioned extracellular stimuli appear to require the activation of the ERK signaling cascade. Blocking the ERK pathway stops their ability to regulate LDL receptor gene expression (Kumar et al., J. Biol. Chem. 275:5214-4221 (1998). ERK belongs to the subfamilies of the mitogen-activated protein kinases (MAPK), the activation of which by successive phosphorylation is secondary to the extracellular stimuli binding to their receptors on cell surface. These receptors either have intrinsic tyrosine kinase activity (like growth factor receptors and insulin receptor) or are coupled to another protein-tyrosine kinase (like receptors for cytokines) (Robinson, Curr Opin. Cell. Biol. 9:180-186 (1997). Upon activation, ERK phosphorylates and activates numerous cytoplasmic or nuclear protein factors, and mediates multiple biological responses including those that control cell growth and differentiation. But how the ERK pathway links to the promoter of the LDL receptor gene and increases its transcription through different mechanisms has not been previously elucidated. In the present invention, as described in Example XI below, it was determined that berberine rapidly activates ERK and that the kinetics of ERK activation preceded the upregulation of LDLR expression by berberine. ERK activation was also determined to be important in berberine's stabilization of LDLR mRNA.

As shown in the examples below, berberine and its analogs exercise post-transcriptional control of the LDL receptor as illustrated in FIG. 2. It simultaneously elevates InsR expression through the PKC system as illustrated in FIG. 34.

Within the methods and compositions of the invention, one or more berberine compound(s), berberine related or derivative compound(s), or proto-berberine compound(s) as disclosed herein is/are additionally effectively formulated or administered as a glucose lowering, insulin resistance decreasing and/or insulin sensitivity increasing compound effective for treating hyperglycemia and/or related disorders. In exemplary embodiments, berberine chloride is demonstrated for illustrative purposes to be a glucose lowering effective agent in pharmaceutical formulations and therapeutic methods, alone or in combination with one or more adjunctive therapeutic agent(s). The present disclosure further provides additional, pharmaceutically acceptable berberine compounds, berberine related and derivative compounds, and proto-berberine compounds in the form of a native or synthetic compound, including complexes, derivatives, including glycosylated derivatives, salts, solvates, isomers, enantiomers, polymorphs, and prodrugs of the compounds disclosed herein, and combinations thereof, which are effective as glucose lowering therapeutic agents within the methods and compositions of the invention.

Hyperglycemia is an abnormally high level of glucose in the blood. It can be caused by disease such as diabetes or may itself cause diabetes, infection, medication, dehydration, lack of exercise, and stress or combinations thereof. It is a classic symptom of diabetes mellitus, but may also be caused by other medical conditions such as obesity. The presence of excessive white fat reserves interferes with the body's ability to properly absorb and use insulin that is otherwise produced in sufficient quantity. Chronic non-diabetic hyperglycemia can produce some of the same complications as diabetic hyperglycemia; however, some of the complications of diabetes mellitus (especially juvenile-onset diabetes mellitus) can occur even if blood sugar levels are kept under control, because the disease operates beyond just the condition of hyperglycemia. The compositions and methods of the present invention are effective in the treatment of all types of hyperglycemia, regardless of cause.

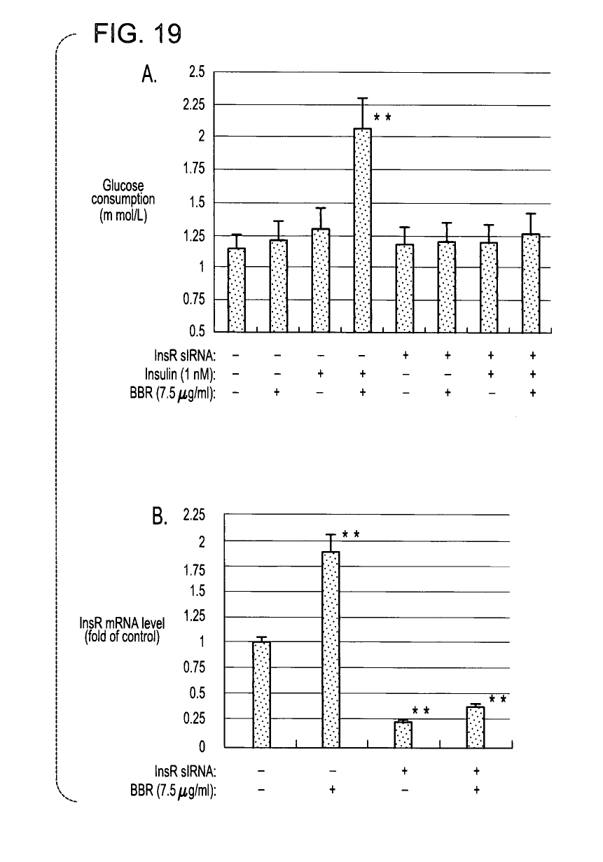

Hyperglycemia may also be caused by insulin resistance. Insulin resistance is a condition in which normal amounts of insulin are inadequate to produce a normal insulin response from fat, muscle and liver cells. To maintain a normal blood glucose, the pancreas secretes additional insulin. When the body cells resist or do not respond to even high levels of insulin, glucose builds up in the blood resulting in high blood glucose. Insulin resistance in fat cells results in hydrolysis of stored triglycerides, which elevates free fatty acids in the blood plasma complicating the control of lipoprotein levels and plaque accumulation. Insulin resistance in muscle reduces glucose uptake whereas insulin resistance in liver reduces glucose storage, with both effects serving to elevate blood glucose. High plasma levels of insulin and glucose due to insulin resistance often lead to the metabolic syndrome and type 2 diabetes. A major contributor to the development of insulin resistance is an overabundance of circulating fatty acids, which are mainly derived from body triglyceride stores. Treatment with berberine compounds of the present invention led to increased InsR expression and an enhanced InsR sensitivity, decreasing insulin resistance and increasing glucose consumption by human hepatic cells (FIG. 19).

Hyperglycemia is a hallmark of diabetes mellitus, a common metabolic disorder resulting from defects in insulin action, insulin production, or both. Symptoms of diabetes include frequent urination, increased thirst, dehydration, weight loss, blurred vision, fatigue, and, occasionally, coma. Diabetes displays an acute symptom due to a remarkably high blood sugar or ketoacidosis, as well as chronic, general metabolic abnormalities arising from a prolonged high blood sugar status or a decrease in glucose tolerance. Both congenital and environmental factors, such as eating habits and exercise, contribute to the disease. Diabetes may be genetic, it may be triggered by infection, both viral and bacterial, autoimmune disease, obesity, exposure to food born illness or chemical toxins, pancreatic disease, and/or treatment with certain pharmaceuticals such as atypical neuroleptics including, but not limited to, olanzepine or risperidol. The pathogenic causes of diabetes are insulin productive disorders, secretion disorders or reductions in activities and sensitivities of the secreted insulin.

Diabetes is largely grouped into the following two types: insulin-dependent diabetes mellitus (also known as Type I diabetes) and non-insulin-dependent but sometimes insulin requiring diabetes mellitus (also known as Type II diabetes). Uncontrolled hyperglycemia over time damages the blood vessels, eyes, nerves, kidneys, and heart, causing organ dysfunction and failure. The underlying metabolic causes of type 2 diabetes, the most common form of diabetes mellitus, are the combination of insulin resistance and defective secretion of insulin by pancreatic β-cells. Insulin resistance develops from obesity and physical inactivity, acting on a substrate of genetic susceptibility. Additionally, insulin secretion declines with advancing age and this decline may be accelerated by genetic factors.

Obesity has been strongly associated with insulin resistance in normoglycemic persons and in individuals with type 2 diabetes. The association of obesity with the insulin resistance syndrome and cardiovascular risk is not only related to the degree of obesity but also seems to be critically dependent on body fat distribution. Thus, individuals with greater degrees of central adiposity develop this syndrome more frequently than do those with a peripheral body fat distribution. In a study of 122 adolescents, obese individuals were significantly more insulin resistant and had an abnormal lipid profile when compared with lean subjects; in this study, insulin resistance was significantly related to an abnormal lipid profile in heavy children but not in thin children, and insulin resistance varied directly with the degree of adiposity. Obesity and insulin resistance have also been shown to be associated with other risk factors, such as elevated blood pressure. (Steinberger, J Pediatr. 126(5 pt 1): 690-695 (1995)). The causes of obesity are myriad and may include psychological, social, and physical components. Obesity may also be caused by treatment with certain pharmaceuticals such as atypical neuroleptics including, but not limited to, olanzepine or risperidol. The compositions and methods of the present invention are effective in the treatment of obesity and related conditions regardless of cause.

One cause of hyperglycemia is the failure of one or more insulin regulatory mechanisms or pathways. Insulin concentrations in plasma are regulated in part by the insulin receptor InsR. InsR is located on the short arm of chromosome 19 (Seino, Proc. Natl. Acad Sci USA 86 (1), 114-118 (1989). Although the coding and promoter region of the human InsR gene has been identified and characterized (Araki J Biol Chem 262, 16186-16191 (1987); Tewari, J. Biol Chem 264 (27) 16238-16245 (1989)) the regulatory mechanisms and pathways controlling InsR gene expression remained to be elucidated.

InsR is composed of two alpha subunits and two beta subunits linked by disulfide bonds. The alpha chains are entirely extracellular and house insulin binding domains, while the linked beta chains penetrate through the plasma membrane. The insulin receptor is a tyrosine kinase functioning as an enzyme that transfers phosphate groups from ATP to tyrosine residues on intracellular target proteins. Binding of insulin to the alpha subunits causes the beta subunits to phosphorylate themselves (autophosphorylation), thus activating the catalytic activity of the receptor and triggering an intracellular insulin pathway that includes InsR activation, Insulin Receptor Substrates (IRS) phosphorylation as well as the cascading trigger of phosphoinositol-3-kinase (P13K), phosphoinositide-dependent kinase (PDK1), protein kinase B (PKB/Akt) and Map kinase. (Saltiel, Cell 104 (4), 517-529 (2001); Kido, J. Clin. Endocrinol Metab, 86 (3), 972-979 (2001)).

In the examples below, it was determined that increased expression of InsR by berberine is operated through a PKC-dependent mechanism separate from berberine's action on LDLR. Berberine upregulates InsR expression through activation of the promoter of InsR gene (transcriptional mechanism) but upregulates LDLR expression by stabilizing LDLR mRNA through an action on the LDLR mRNA 3′UTR region (post-transcriptional mechanism). The berberine caused increase in InsR expression was observed in muscle tissue and lymphocytes as well as liver cells.

PKC is a family of phopholipid-dependent serine/threonine kinases that transduce a wide range of biological signals including those related to the insulin pathway. Atypical PKCs (ζ and λ) are downstream events of insulin stimulation (Farese, Am J Physiol Endocrinol Metab 283 (1), E1-11 (2002). In the examples below, PKC inhibitor calphostin C eliminated the stimulating effect of berberine on the promoter of the InsR gene and InsR mRNA transcription, indicating that PKC is required for the effect of berberine on InsR gene transcription. The examples further demonstrate that PKC is a part of the activation mechanism for the InsR gene promoter.

The elevated expression of InsR on the cell surface served as the primary mechanism of berberine for the restoration of insulin sensitivity in vivo. The upregulatory effect of berberine on InsR expression was clearly observed in liver tissue of hyperglycemic rats, and correlated with the reduction of blood glucose. Additionally, increased InsR expression directly translated to enhanced InsR sensitivity in target cells increasing glucose consumption on human hepatic cells treated with insulin. (FIG. 19)

In the clinical study conducted in patients with type 2 diabetes, a reduction of blood glucose of 26% and HbAc1 of 18% was achieved after two months of treatment with berberine. In addition, the 18% reduction of serum triglyceride in these patients reflects at least partially an improved glycogen synthesis from the glucose pool. Since berberine also lowers serum lipids that influence sugar metabolism, the reduction of glucose and triglycerides in the circulation represents a synergistic effect of berberine on the activation of both InsR and LDLR expression. As illustrated in FIG. 34, berberine increases the LDLR expression through activation of ERK pathway in lever cells, and also elevates InsR expression through the PKC system. The two signal pathways closely collaborate in producing a full cellular response against lipid/glucose related metabolic disorders. In the study described in the examples below, 50% of the type 2 diabetes patients in the berberine treatment group also had hyperlipidemia. Treatment with berberine not only reduced the blood glucose, but also reduced serum cholesterol by 24%. Additionally, as shown in FIG. 36, the percentage of lymphocytes expressing InsR on the surface significantly increased after treatment with berberine.

Essential hypertension is the clinical expression of a disordered interaction between the genetic, physiological, and biochemical systems that under usual conditions maintain cardiovascular homeostasis. The multifactorial nature of essential hypertension has made it difficult to completely isolate the action of any one of these systems from the actions of the others. The relation between insulin metabolism/resistance and essential hypertension has the potential to provide insight into the mechanisms that operate this complex interaction. (DeFronzo, Diabetes Care 14: 173-194 (1991) Insulin increases renal sodium retention while increasing free water clearance. Insulin resistance is also associated with increased sympathetic nervous system activity and stimulation of vascular smooth muscle growth. Additionally, insulin levels have been found to be significantly higher in adult patients with essential hypertension and borderline hypertension than in normotensive control patients. This is true regardless of the technique used to measure insulin and glucose level and independent of age, sex, and ethnic group. Numerous studies have confirmed the association between weight gain, percent body fat, and insulin resistance. However, there has also been found to be an interaction between insulin and hypertension that is independent of obesity. (Steinberger, Circulation. 107:1448 (2003). Treatment with berberine compounds of the present invention is effective in reducing hypertension, regardless of the cause.

Metabolic syndrome also known as syndrome X, dysmetabolic syndrome, obesity syndrome, insulin resistance syndrome and Reaven's syndrome, is a collection of risk factors estimated to effect over 50 million Americans. While there are no well-accepted criteria for diagnosing metabolic syndrome, it is generally characterized by abdominal obesity, atherogenic dislipidemia, elevated blood pressure, insulin resistance or glucose intolerance, prothrombotic state and a proinflammatory state. People with metabolic syndrome are at increased risk of coronary heart disease, stroke, peripheral vascular diseases, fatty liver, skin lesions, reproductive abnormalities, growth abnormalities, type 2 diabetes, and accelerated atherosclerosis as well as other diseases related to the buildup of arterial plaques formed by lipoproteins. Treatment with berberine compounds of the present invention is effective in treating metabolic syndrome, regardless of cause.

Berberine is a quaternary alkaloid widely distributed in nine plant families of the structure of the compound of formula II.

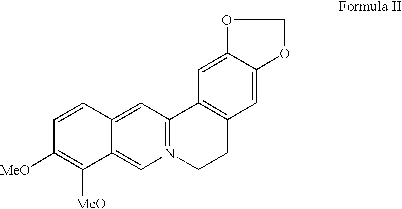

Berberine can be found in Hydrastis canadensis (goldenseal), Coptis chinensis (Coptis, goldenthread, also known as the Chinese herb Huanglian), Berberis aquifolium (Oregon grape), Berberis vulgaris (barberry), Berberis aristata (tree turmeric), Chinese Isatis, Mahonia swaseyi, Yerba mansa (Anemopsis californica), and Phellodendron amurense. Products from these and other berberine-containing herbal sources, including any preparation or extract therefrom, are contemplated as useful compositions comprising berberine (or berberine analogs, related compounds, proto-berberine compounds and/or derivatives) for use within the invention. Useful berberine compounds, berberine related, proto-berberine and derivative compounds for use within the invention will typically have a structure as illustrated in Formula I, although functionally equivalent analogs, complexes, conjugates, and derivatives of such compounds will also be appreciated by those skilled in the art as within the scope of at least certain aspects of this invention.

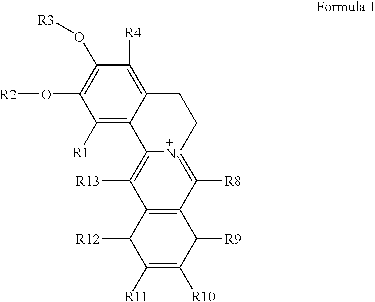

Useful berberine compounds, berberine related, proto-berberine and derivative compounds for use within the invention according to Formula I will also typically have a structure wherein R₁, R₂, R₃, R₄, R₈, R₉, R₁₀, R₁₁, R₁₂ and/or R₁₃ is selected (each independently, and in any combination yielding an active compound as described) from a hydrogen, halogen, hydroxy, alkyl, alkoxy, nitro, amino, trifluoromethyl, cycloalkyl, (cycloalkyl)alkyl, alkanoyl, alkanoyloxy, aryl, aroyl, aralkyl, nitrile, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, alkanoylamino, carbamoyl, carbamyl, carbonylamino, alkylsulfonylamino, oligosaccharide or heterocyclo group.

In more detailed embodiments, illustrative structural modifications according to Formula I above will be selected to provide useful candidate compounds for treating and/or preventing hyperlipidemia in mammalian subjects wherein: R₁ is selected from methyl, ethyl, hydroxyl, or methoxy; R₂ is selected from H, methyl, ethyl, methene; R₃ is selected from H, methyl, ethyl, methene; R₄ is selected from a hydrogen atom, methyl, ethyl, hydroxyl, or methoxy, an alkyl group having 1 to 8 carbons, or an alkenyl group having 3 to 8 carbons; R₈ is selected from straight or branched (C1-C6)alkyl (e.g., substitution selected from methyl, ethyl, n-propyl, 1-methylethyl, n-butyl, 1-methylpropyl, 2-methylpropyl, 1,1-dimethylethyl, n-pentyl, 2-methylbutyl, 1,1-dimethylpropyl, 2,2 dimethylpropyl, 3-methylbutyl, n-hexyl, 1-methylpentyl, 1,1-dimethylbutyl, 2,2-dimethylbutyl, 3-methylpentyl, 1,2-dimethylbutyl, 1,3-dimethyl and 1-methyl-2ethylpropyl); R₉ is selected from methyl, ethyl, hydroxyl, Cl, Br; R₁₀ is selected from methyl, ethyl, hydroxyl, Cl, Br, hydroxy or an alkoxy group having 1 to 4 carbons; R₁₁ is selected from methyl, ethyl, hydroxyl, Cl, Br; R₁₂ is selected from methyl, ethyl, hydroxyl, Cl, Br; and R₁₃ is selected from straight or branched (C₁-C₈)alkyl (e.g., substitution selected from methyl, ethyl, n-propyl, 1-methylethyl, n-butyl, 1-methylpropyl, 2-methylpropyl, 1,1-dimethylethyl, n-pentyl, 2-methylbutyl, 1,1-dimethylpropyl, 2,2 dimethylpropyl, 3-methylbutyl, n-hexyl, 1-methylpentyl, 1,1-dimethylbutyl, 2,2-dimethylbutyl, 3-methylpentyl, 1,2-dimethylbutyl, 1,3-dimethyl and 1-methyl-2ethylpropyl), hydrogen atom, an alkenyl group having 3 to 8 carbon atoms, a cycloalkylalkyl group having 1 to 7 carbon atoms, a holoalkyl group having 1 to 4 carbon atoms, an ethoxycarbonyl group, an ethoxycarbonylmethyl group, a hydroxycarbonylmethyl group, 1-ethoxycarbonylethyl group, or 2-valerolactonyl group. Yet additional candidate compounds for use within the compositions and methods of the invention are provided wherein each of the R₁, R₂, R₃, R₄, R₈, R₉, R₁₀, R₁₁, R₁₂, and/or R₁₃ groups indicated in Formula I can be optionally (independently, collectively, or in any combination yielding an active compound as described) substituted as described and defined in the following passages.

In additional detailed embodiments, illustrative structural modifications according to Formula I above will be selected to provide useful candidate compounds for treating and/or preventing hyperglycemia, insulin resistance, obesity, diabetes, metabolic syndrome and hypertension in mammalian subjects wherein: R₁ is selected from methyl, ethyl, hydroxyl, or methoxy; R₂ is selected from H, methyl, ethyl, methene; R₃ is selected from H, methyl, ethyl, methene; R₄ is selected from methyl, ethyl, hydroxyl, or methoxy; R₈ is selected from straight or branched (C1-C6)alkyl (e.g., substitution selected from methyl, ethyl, n-propyl, 1-methylethyl, n-butyl, 1-methylpropyl, 2-methylpropyl, 1,1-dimethylethyl, n-pentyl, 2-methylbutyl, 1,1-dimethylpropyl, 2,2 dimethylpropyl, 3-methylbutyl, n-hexyl, 1-methylpentyl, 1,1-dimethylbutyl, 2,2-dimethylbutyl, 3-methylpentyl, 1,2-dimethylbutyl, 1,3-dimethyl and 1-methyl-2ethylpropyl); R₉ is selected from methyl, ethyl, hydroxyl, Cl, Br; R₁₀ is selected from methyl, ethyl, hydroxyl, Cl, Br; R₁₁ is selected from methyl, ethyl, hydroxyl, Cl, Br; R₁₂ is selected from methyl, ethyl, hydroxyl, Cl, Br; and R₁₃ is selected from straight or branched (C1-C6)alkyl (e.g., substitution selected from methyl, ethyl, n-propyl, 1-methylethyl, n-butyl, 1-methylpropyl, 2-methylpropyl, 1,1-dimethylethyl, n-pentyl, 2-methylbutyl, 1,1-dimethylpropyl, 2,2 dimethylpropyl, 3-methylbutyl, n-hexyl, 1-methylpentyl, 1,1-dimethylbutyl, 2,2-dimethylbutyl, 3-methylpentyl, 1,2-dimethylbutyl, 1,3-dimethyl and 1-methyl-2ethylpropyl). Yet additional candidate compounds for use within the compositions and methods of the invention are provided wherein each of the R₁, R₂, R₃, R₄, R₈, R₉, R₁₀, R₁₁, and/or R₁₂ groups indicated in Formula I can be optionally (independently, collectively, or in any combination yielding an active compound as described) substituted as described and defined in the following passages.

The term “halogen” as used herein refers to bromine, chlorine, fluorine or iodine. In one embodiment, the halogen is fluorine. In another embodiment, R₉, R₁₀, R₁₁, R₁₂ and/or R₁₃ may independently be chlorine or bromine.

The term “hydroxy” as used herein refers to —OH or —O⁻.

The term “alkene” as used herein refers to unsaturated hydrocarbons that contain carbon-carbon double bonds. Examples of such alkene groups include ethylene, propene, and the like. In one embodiment, R₂ and/or R₃ may independently be methene.

The term “alkyl” as used herein refers to straight- or branched-chain aliphatic groups containing 1-20 carbon atoms, preferably 1-7 carbon atoms and most preferably 1-6 carbon atoms. This definition applies as well to the alkyl portion of alkoxy, alkanoyl and aralkyl groups. In one embodiment, R₁, R₂, R₃, R₄, R₈ and/or R₁₃ may independently be methyl or ethyl groups. In another embodiment R₈ and/or R₁₃ may independently be n-propyl, 1-methylethyl, n-butyl, 1-methylpropyl, 2-methylpropyl, 1,1 dimethyleledhyl, n-pentyl, 2-methylbutyl, 1,1-dimethylpropyl, 2,2-dimethylpropyl, 3-methylbutyl, m-hexyl, 1-methylpentyl, 1,1-dimethylbutyl, 2,2-dimethylbutyl, 3-methylpentyl, 1-2-dimethylbutyl, 1,3-dimethyl or 1-methyl-2ethylpropyl.

The term “alkoxy” includes substituted and unsubstituted alkyl, alkenyl, and alkynyl groups covalently linked to an oxygen atom. In one embodiment, the alkoxy group contains 1 to 6 carbon atoms. Embodiments of alkoxy groups include, but are not limited to, methoxy, ethoxy, isopropyloxy, propoxy, butoxy, and pentoxy groups. In a further embodiment, R₉, R₁₀, R¹¹, and/or R₁₂ may independently be methoxy or ethoxy groups. In another embodiment, R₁ is a methoxy group. Embodiments of substituted alkoxy groups include halogenated alkoxy groups. In a further embodiment, the alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Exemplary halogen substituted alkoxy groups include, but are not limited to, fluoromethoxy, difluoromethoxy, trifluoromethoxy, chloromethoxy, dichloromethoxy, and trichloromethoxy. In one embodiment, R₁, R₄, R₉, R₁₀, R₁₁ and/or R₁₂ may independently be an hydroxyl group.

The term “nitro,” as used herein alone or in combination refers to a—NO₂ group.

The term “amino” as used herein refers to the group —NRR′, where R and R′ may independently be hydrogen, alkyl, aryl, alkoxy, or heteroaryl. The term “aminoalkyl” as used herein represents a more detailed selection as compared to “amino” and refers to the group —NRR′, where R and R′ may independently be hydrogen or (C₁-C₄)alkyl.

The term “trifluoromethyl” as used herein refers to —CF₃.

The term “trifluoromethoxy” as used herein refers to —OCF₃.

The term “cycloalkyl” as used herein refers to a saturated cyclic hydrocarbon ring system containing from 3 to 7 carbon atoms that may be optionally substituted. Exemplary embodiments include, but are not limited to, cyclopropyl, cyclobutyl, cyclopentyl and cyclohexyl. In certain embodiments, the cycloalkyl group is cyclopropyl. In another embodiment, the (cycloalkyl)alkyl groups contain from 3 to 7 carbon atoms in the cyclic portion and 1 to 4 carbon atoms in the alkyl portion. In certain embodiments, the (cycloalkyl)alkyl group is cyclopropylmethyl. The alkyl groups are optionally substituted with from one to three substituents selected from the group consisting of halogen, hydroxy and amino.

The terms “alkanoyl” and “alkanoyloxy” as used herein refer, respectively, to —C(O)-alkyl groups and —O—C(O)-alkyl groups, each optionally containing 2-5 carbon atoms. Specific embodiments of alkanoyl and alkanoyloxy groups are acetyl and acetoxy, respectively.

The term “aryl” as used herein refers to monocyclic or bicyclic aromatic hydrocarbon groups having from 6 to 12 carbon atoms in the ring portion, for example, phenyl, naphthyl, biphenyl and diphenyl groups, each of which may be substituted with, for example, one to four substituents such as alkyl; substituted alkyl as defined above, halogen, trifluoromethyl, trifluoromethoxy, hydroxy, alkoxy, cycloalkyloxy, alkanoyl, alkanoyloxy, amino, alkylamino, dialkylamino, nitro, cyano, carboxy, carboxyalkyl, carbamyl, carbamoyl and aryloxy. Specific embodiments of aryl groups in accordance with the present invention include phenyl, substituted phenyl, naphthyl, biphenyl, and diphenyl.

The term “aroyl” as used herein refers to an aryl radical derived from an aromatic carboxylic acid, such as optionally substituted benzoic or naphthoic acids.

The term “nitrile” or “cyano” as used herein refers to the group —CN.

The term “dialkylamino” refers to an amino group having two attached alkyl groups that can be the same or different.

The term “alkenyl” refers to a straight or branched alkenyl group of 2 to 10 carbon atoms having 1 to 3 double bonds. Preferred embodiments include ethenyl, 1-propenyl, 2-propenyl, 1-methylethenyl, 1-butenyl, 2-butenyl, 3-butenyl, 2-methyl-2-propenyl, 1-pentenyl, 2-pentenyl, 4-pentenyl, 3-methyl-2-butenyl, 1-hexenyl, 2-hexenyl, 1-heptenyl, 2-heptenyl, 1-octenyl, 2-octenyl, 1,3-octadienyl, 2-nonenyl, 1,3-nonadienyl, 2-decenyl, etc.

The term “alkynyl” as used herein refers to a straight or branched alkynyl group of 2 to 10 carbon atoms having 1 to 3 triple bonds. Exemplary alkynyls include, but are not limited to, ethynyl, 1-propynyl, 2-propynyl, 1-butynyl, 2-butynyl, 3-butynyl, 1-pentynyl, 2-pentynyl, 4-pentynyl, 1-octynyl, 6-methyl-1-heptynyl, and 2-decynyl.

The term “hydroxyalkyl” alone or in combination, refers to an alkyl group as previously defined, wherein one or several hydrogen atoms, preferably one hydrogen atom has been replaced by a hydroxyl group. Examples include hydroxymethyl, hydroxyethyl and 2-hydroxyethyl.

The term “aminoalkyl” as used herein refers to the group —NRR′, where R and R′ may independently be hydrogen or (C1-C6)alkyl.

The term “alkylaminoalkyl” refers to an alkylamino group linked via an alkyl group (i.e., a group having the general structure -alkyl-NH-alkyl or -alkyl-N(alkyl)(alkyl)). Such groups include, but are not limited to, mono- and di-(C₁-C₈ alkyl)aminoC₁-C₈ alkyl, in which each alkyl may be the same or different.

The term “dialkylaminoalkyl” refers to alkylamino groups attached to an alkyl group. Examples include, but are not limited to, N,N-dimethylaminomethyl, N,N-dimethylaminoethyl N,N-dimethylaminopropyl, and the like. The term dialkylaminoalkyl also includes groups where the bridging alkyl moiety is optionally substituted.

The term “haloalkyl” refers to an alkyl group substituted with one or more halo groups, for example chloromethyl, 2-bromoethyl, 3-iodopropyl, trifluoromethyl, perfluoropropyl, 8-chlorononyl and the like.

The term “carboxyalkyl” as used herein refers to the substituent —R′—COOH wherein R′ is alkylene; and carbalkoxyalkyl refers to —R′—COOR wherein R′ and R are alkylene and alkyl respectively. In certain embodiments, alkyl refers to a saturated straight- or branched-chain hydrocarbyl radical of 1-6 carbon atoms such as methyl, ethyl, n-propyl, isopropyl, n-butyl, t-butyl, n-pentyl, 2-methylpentyl, n-hexyl, and so forth. Alkylene is the same as alkyl except that the group is divalent.

The term “alkoxyalkyl” refers to a alkylene group substituted with an alkoxy group. For example, methoxyethyl [CH₃OCH₂CH₂—] and ethoxymethyl (CH₃CH₂OCH₂—] are both C₃ alkoxyalkyl groups.

The term “carboxy”, as used herein, represents a group of the formula —COOH.

The term “alkanoylamino” refers to alkyl, alkenyl or alkynyl groups containing the group —C(O)— followed by —N(H)—, for example acetylamino, propanoylamino and butanoylamino and the like.

The term “carbonylamino” refers to the group —NR—CO—CH₂—R′, where R and R′ may be independently selected from hydrogen or (C₁-C₄)alkyl.

The term “carbamoyl” as used herein refers to —O—C(O)NH₂.

The term “carbamyl” as used herein refers to a functional group in which a nitrogen atom is directly bonded to a carbonyl, i.e., as in —NRC(═O)R′ or —C(═O)NRR′, wherein R and R′ can be hydrogen, alkyl, substituted alkyl, alkenyl, substituted alkenyl, alkoxy, cycloalkyl, aryl, heterocyclo, or heteroaryl.

The term “alkylsulfonylamino” refers to refers to the group —NHS(O)₂Rₐ wherein Rₐ is an alkyl as defined above.

The term “heterocyclo” refers to an optionally substituted, unsaturated, partially saturated, or fully saturated, aromatic or nonaromatic cyclic group that is a 4 to 7 membered monocyclic, or 7 to 11 membered bicyclic ring system that has at least one heteroatom in at least one carbon atom-containing ring. The substituents on the heterocyclo rings may be selected from those given above for the aryl groups. Each ring of the heterocyclo group containing a heteroatom may have 1, 2 or 3 heteroatoms selected from nitrogen atoms, oxygen atoms and sulfur atoms. Plural heteroatoms in a given heterocyclo ring may be the same or different.

The term “furyl” refers to a heterocyclic group, having the formula C₄H₃O, which may be either the alpha or beta isomer

As used herein, the term “benzotriazolyl” refers to a monovalent group having a benzene group fused to a triazolyl group. The formula for a benzotriazolyl group is C₆H₄N₃—.

The term “benzyloxy” refers to an O—CH₂Ph substituent, wherein Ph is phenyl or a substituted phenyl.

The term “methylenedioxy” refers to a —O—CH₂—O— group.

The term “vinyl” refers to the group CH₂CH.

The term “glycosylate,” “glycosylation” or “glycosylated means the attachment of an oligosaccharide group, preferably, though not limited to, attachment to an nitrogen or oxygen.

The term “oligosaccharide” as used herein is defined as encompassing 1 to 20 saccharides. Mono-, di-, and trisaccharides are specifically included in the definition of oligosaccharides.

All value ranges expressed herein, are inclusive over the indicated range. Thus, a range of R between 0 to 4 will be understood to include the values of 1, 2, 3, and 4.

In some embodiments, derivative forms of the berberine compound of formula II may be formed through demethylation. In other embodiments, the berberine compound of formula II may be hydrogenated, in further embodiments, the berberine compound of formula II may be demethylated and hydrogenated such that it forms compounds such as or derived from canadine of the structure of formula III below.

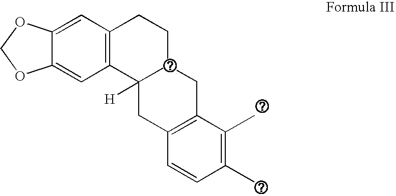

Exemplary forms of berberine compounds and derivatives of formula I may be found in table 2 below.

TABLE 2
Exemplary forms of berberine compounds and derivatives of formula I

| Composition | | | | | | | | | | |
|---|---|---|---|---|---|---|---|---|---|---|
| No. | R¹ | R² | R³ | R⁴ | R⁸ | R⁹ | R¹⁰ | R¹¹ | R¹² | R¹³ |
| 1 | H | —CH₂— | | H | 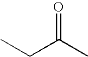 | CH₃O | CH₃O | H | H | H |
| 2 | H | —CH₂— | | H | CH₃ | CH₃O | CH₃O | H | H | H |
| 3 | H | —CH₂— | | H | C₂H₅ | CH₃O | CH₃O | H | H | H |
| 4 | H | —CH₂— | | H | n-Pr | CH₃O | CH₃O | H | H | H |
| 5 | H | —CH₂— | | H | n-Bu | CH₃O | CH₃O | H | H | H |
| 6 | H | —CH₂— | | H | 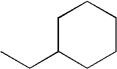 | CH₃O | CH₃O | H | H | H |
| 7 | H | —CH₂— | | H | i-Pr | CH₃O | CH₃O | H | H | H |
| 8 | H | CH₃ | CH₃ | H | CH₃ | CH₃O | CH₃O | H | H | H |
| 9 | H | CH₃ | CH₃ | H | C₂H₅ | CH₃O | CH₃O | H | H | H |
| 10 | H | —CH₂— | | H | noctyl | CH₃O | CH₃O | H | H | H |
| 11 | H | —CH₂— | | H | 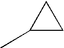 | CH₃O | CH₃O | H | H | H |
| 12 | H | CH₃ | CH₃ | H | 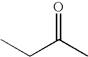 | CH₃O | CH₃O | H | H | C₂H₅ |
| 13 | H | CH₃ | CH₃ | H | 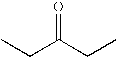 | CH₃O | CH₃O | H | H | C₂H₅ |
| 14 | H | CH₃ | CH₃ | H | 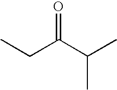 | CH₃O | CH₃O | H | H | C₂H₅ |
| 15 | H | CH₃ | CH₃ | H | 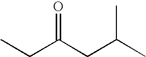 | CH₃O | CH₃O | H | H | C₂H₅ |
| 16 | H | CH₃ | CH₃ | H | 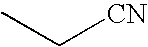 | CH₃O | CH₃O | H | H | C₂H₅ |
| 17 | H | CH₃ | CH₃ | H | 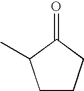 | CH₃O | CH₃O | H | H | C₂H₅ |
| 18 | H | CH₃ | CH₃ | H | 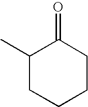 | CH₃O | CH₃O | H | H | C₂H₅ |
| 19 | H | C₂H₅ | C₂H₅ | H | 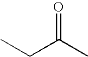 | C₂H₅O | C₂H₅O | H | H | C₂H₅ |
| 20 | H | C₂H₅ | C₂H₅ | H | 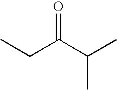 | C₂H₅O | C₂H₅O | H | H | C₂H₅ |
| 21 | H | CH₃ | CH₃ | H | H | CH₃O | CH₃O | H | H | C₂H₅ |
| 22 | H | C₂H₅ | C₂H₅ | H | H | C₂H₅O | C₂H₅O | H | H | C₂H₅ |
| 23 | H | CH₃ | CH₃ | H | CH₃ | CH₃O | CH₃O | H | H | C₂H₅ |
| 24 | H | CH₃ | CH₃ | H | C₂H₅ | CH₃O | CH₃O | H | H | C₂H₅ |
| 25 | H | CH₃ | CH₃ | H | n-Bu | CH₃O | CH₃O | H | H | C₂H₅ |

Additional exemplary forms of berberine compounds and derivatives contemplated for use within the methods and compositions of the invention may additionally have the structure of formula IV, below.

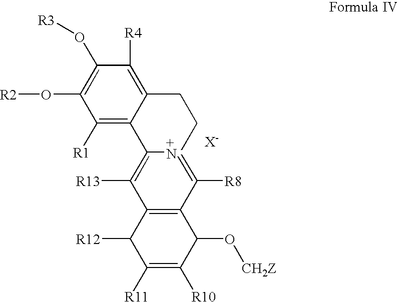

Wherein X represents an inorganic acid ion, organic acid ion, or halide, more particularly, nitrate, sulfate, acetate, tartrate, maleate, succinate, citrate, fumarate, aspartate, salicylate, glycerate, ascorbate, fluoride, chloride, iodide or bromide. Z represents an alkyl group having 5 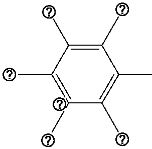
to 12 carbons, or an alkenyl group having 4 to 6 carbons, a N-benzotriazolyl group, a quinolinyl group, a furyl group, a substituted furyl group, or a radical represented by the formula:

wherein Z¹, Z², Z³, Z⁴ and Z⁵ which may be the same or different from each other, represent a hydrogen atom, halogen, an alkyl group having 1 to 5 carbons, a trifluoromethyl group, a phenyl group, a substituted phenyl group, a nitro group, an alkoxy group having 1 to 4 carbons, a methylenedioxy group, a trifluoro-methoxy group, a hydroxy group, a benzyloxy group, a phenoxy group, a vinyl group, a benzenesulfonylmethyl group or a methoxycarbonyl group; and A and B which may also be the same or different from each other, represent carbon or nitrogen. Berberine compounds and derivatives of Formula IV are exemplified by the compounds in the table 3 below.

TABLE 3
Exemplary compositions of berberine compounds and derivatives of formula IV.

| Composition | | | | | | | | | | | |
|---|---|---|---|---|---|---|---|---|---|---|---|
| No. | R¹ | R² | R³ | R⁴ | R⁸ | R¹⁰ | R¹¹ | R¹² | R¹³ | Z | X |
| 26 | H | CH₃ | CH₃ | H | H | OCH₃ | H | H | C₂H₅ | CH₃(CH₂)₁₀CH₃ | Cl |
| 27 | H | CH₃ | CH₃ | H | H | OCH₃ | H | H | C₂H₅ | 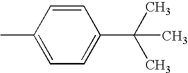 | Cl |
| 28 | H | CH₃ | CH₃ | H | H | OCH₃ | H | H | C₂H₅ | 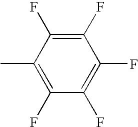 | Cl |
| 29 | H | CH₃ | CH₃ | H | H | OCH₃ | H | H | C₂H₅ | 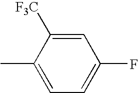 | Cl |
| 30 | H | CH₃ | CH₃ | H | H | OCH₃ | H | H | C₂H₅ | 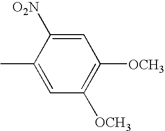 | Cl |
| 31 | H | CH₃ | CH₃ | H | H | OCH₃ | H | H | C₂H₅ | 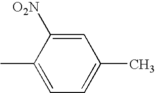 | Cl |
| 32 | H | CH₃ | CH₃ | H | H | OCH₃ | H | H | C₂H₅ | 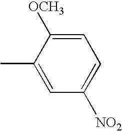 | Cl |
| 33 | H | CH₃ | CH₃ | H | H | OCH₃ | H | H | C₂H₅ | 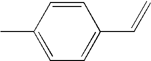 | Cl |
| 34 | H | CH₃ | CH₃ | H | H | OCH₃ | H | H | C₂H₅ | 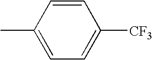 | Cl |
| 35 | H | CH₃ | CH₃ | H | H | OCH₃ | H | H | C₂H₅ | 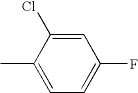 | Cl |
| 36 | H | CH₃ | CH₃ | H | H | OCH₃ | H | H | C₂H₅ | 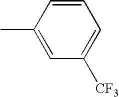 | Cl |
| 37 | H | CH₃ | CH₃ | H | H | OCH₃ | H | H | C₂H₅ | 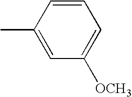 | Cl |
| 38 | H | CH₃ | CH₃ | H | H | OCH₃ | H | H | C₂H₅ | 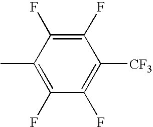 | Cl |
| 39 | H | CH₃ | CH₃ | H | H | OCH₃ | H | H | C₂H₅ | 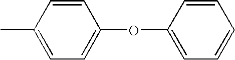 | Cl |
| 40 | H | CH₃ | CH₃ | H | H | OCH₃ | H | H | C₂H₅ | 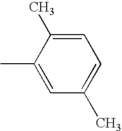 | Cl |
| 41 | H | CH₃ | CH₃ | H | H | OCH₃ | H | H | C₂H₅ | 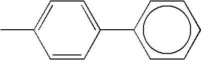 | Cl |
| 42 | H | CH₃ | CH₃ | H | H | OCH₃ | H | H | C₂H₅ | 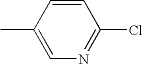 | Cl |
| 43 | H | CH₃ | CH₃ | H | H | OCH₃ | H | H | C₂H₅ | 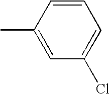 | Cl |
| 44 | H | CH₃ | CH₃ | H | H | OCH₃ | H | H | C₂H₅ | 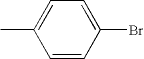 | Cl |
| 45 | H | CH₃ | CH₃ | H | H | OCH₃ | H | H | C₂H₅ | 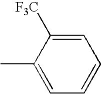 | Cl |
| 46 | H | CH₃ | CH₃ | H | H | OCH₃ | H | H | C₂H₅ | 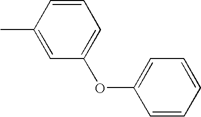 | Cl |
| 47 | H | CH₃ | CH₃ | H | H | OCH₃ | H | H | C₂H₅ | 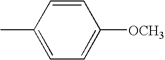 | Cl |
| 48 | H | CH₃ | CH₃ | H | H | OCH₃ | H | H | C₂H₅ | 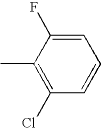 | Cl |
| 49 | H | CH₃ | CH₃ | H | H | OCH₃ | H | H | C₂H₅ | 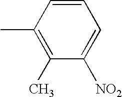 | Cl |
| 50 | H | CH₃ | CH₃ | H | H | OCH₃ | H | H | C₂H₅ | 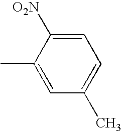 | Cl |
| 51 | H | CH₃ | CH₃ | H | H | OCH₃ | H | H | C₂H₅ | —CH═CH₂ | Cl |
| 52 | H | CH₃ | CH₃ | H | H | OCH₃ | H | H | C₂H₅ | 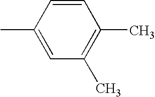 | Cl |
| 53 | H | CH₃ | CH₃ | H | H | OCH₃ | H | H | C₂H₅ | 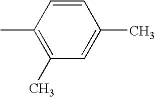 | Cl |
| 54 | H | CH₃ | CH₃ | H | H | OCH₃ | H | H | C₂H₅ | 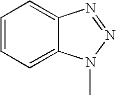 | Cl |
| 55 | H | CH₃ | CH₃ | H | H | OCH₃ | H | H | C₂H₅ | 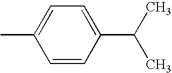 | Cl |
| 56 | H | CH₃ | CH₃ | H | H | OCH₃ | H | H | C₂H₅ | 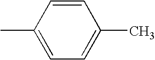 | Cl |
| 57 | H | CH₃ | CH₃ | H | H | OCH₃ | H | H | C₂H₅ | 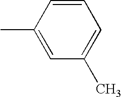 | Cl |
| 58 | H | CH₃ | CH₃ | H | H | OCH₃ | H | H | C₂H₅ | 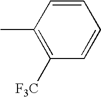 | Cl |
| 59 | H | CH₃ | CH₃ | H | H | OCH₃ | H | H | C₂H₅ | 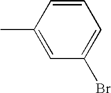 | Cl |
| 60 | H | CH₃ | CH₃ | H | H | OCH₃ | H | H | C₂H₅ | 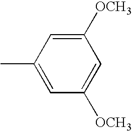 | Cl |
| 61 | H | CH₃ | CH₃ | H | H | OCH₃ | H | H | C₂H₅ | 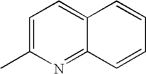 | Cl |
| 62 | H | CH₃ | CH₃ | H | H | OCH₃ | H | H | C₂H₅ | 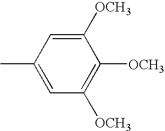 | Cl |
| 63 | H | CH₃ | CH₃ | H | H | OCH₃ | H | H | C₂H₅ | 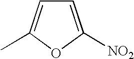 | Cl |
| 64 | H | CH₃ | CH₃ | H | H | OCH₃ | H | H | C₂H₅ | 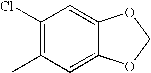 | Cl |
| 65 | H | CH₃ | CH₃ | H | H | OCH₃ | H | H | C₂H₅ | 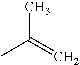 | Cl |
| 66 | H | CH₃ | CH₃ | H | H | OCH₃ | H | H | C₂H₅ | 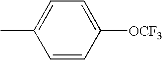 | Cl |
| 67 | H | CH₃ | CH₃ | H | H | OCH₃ | H | H | C₂H₅ | 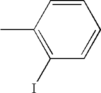 | Cl |
| 68 | H | CH₃ | CH₃ | H | H | OCH₃ | H | H | C₂H₅ | 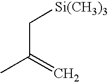 | Cl |
| 69 | H | —CH₂— | | H | H | OCH₃ | H | H | C₂H₅ | —CH₂(CH₂)₁₀CH₃ | I |
| 70 | H | —CH₂— | | H | H | OCH₃ | H | H | C₂H₅ | 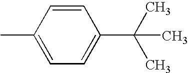 | I |
| 71 | H | —CH₂— | | H | H | OCH₃ | H | H | C₂H₅ | 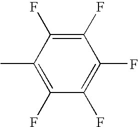 | I |
| 72 | H | —CH₂— | | H | H | OCH₃ | H | H | C₂H₅ | 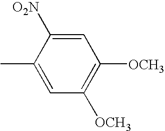 | I |
| 73 | H | —CH₂— | | H | H | OCH₃ | H | H | C₂H₅ | 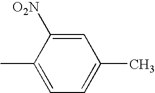 | I |
| 74 | H | —CH₂— | | H | H | OCH₃ | H | H | C₂H₅ | 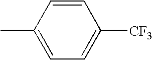 | I |
| 75 | H | —CH₂— | | H | H | OCH₃ | H | H | C₂H₅ | 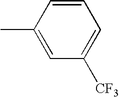 | I |
| 76 | H | —CH₂— | | H | H | OCH₃ | H | H | C₂H₅ | 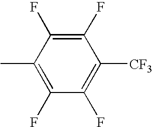 | I |
| 77 | H | —CH₂— | | H | H | OCH₃ | H | H | C₂H₅ | 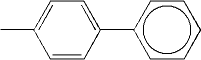 | I |
| 78 | H | —CH₂— | | H | H | OCH₃ | H | H | C₂H₅ | 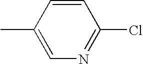 | I |
| 79 | H | C₂H₅ | C₂H₅ | H | H | OCH₃ | H | H | C₂H₅ | 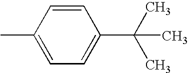 | Cl |
| 80 | H | C₂H₅ | C₂H₅ | H | H | OCH₃ | H | H | C₂H₅ | 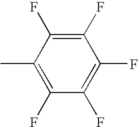 | Cl |
| 81 | H | C₂H₅ | C₂H₅ | H | H | OCH₃ | H | H | C₂H₅ | 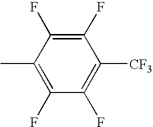 | Cl |
| 82 | H | C₂H₅ | C₂H₅ | H | H | OCH₃ | H | H | C₂H₅ | 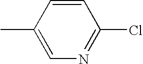 | Cl |
| 83 | H | H | H | H | H | OCH₃ | H | H | C₂H₅ | 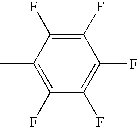 | Cl |
| 84 | H | H | H | H | H | OCH₃ | H | H | C₂H₅ | 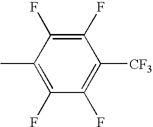 | Cl |
| 85 | H | C₂H₅ | C₂H₅ | H | H | OC₂H₅ | H | H | C₂H₅ | —CH₂(CH₂)₁₀CH₃ | Cl |
| 86 | H | C₂H₅ | C₂H₅ | H | H | OC₂H₅ | H | H | C₂H₅ | 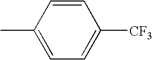 | Cl |
| 87 | H | C₂H₅ | C₂H₅ | H | H | OC₂H₅ | H | H | C₂H₅ | 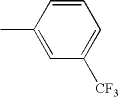 | Cl |
| 88 | H | C₂H₅ | C₂H₅ | H | H | OC₂H₅ | H | H | C₂H₅ | 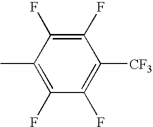 | Cl |
| 89 | H | C₂H₅ | C₂H₅ | H | H | OC₂H₅ | H | H | C₂H₅ | 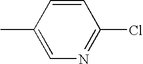 | Cl |
| 90 | H | C₃H₇ | C₃H₇ | H | H | OC₃H₇ | H | H | C₂H₅ | 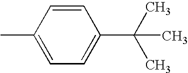 | Cl |
| 91 | H | C₃H₇ | C₃H₇ | H | H | OC₃H₇ | H | H | C₂H₅ | 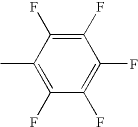 | Cl |
| 92 | H | C₃H₇ | C₃H₇ | H | H | OC₃H₇ | H | H | C₂H₅ | 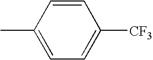 | Cl |
| 93 | H | CH₃ | CH₃ | H | H | OCH₃ | H | H | C₂H₅ | 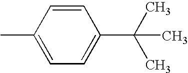 | HSO₄⁻ |
| 94 | H | CH₃ | CH₃ | H | H | OCH₃ | H | H | C₂H₅ | 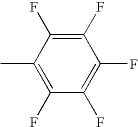 | CH₃CO₂⁻ |
| 95 | H | CH₃ | CH₃ | H | H | OCH₃ | H | H | C₂H₅ | 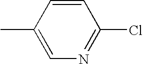 | NO₃⁻ |

Ionized forms of berberine, such as berberine chloride, are also contemplated for use within the methods and compositions of the invention. Berberine chloride exemplifies this type of compound having the structure of formula V below.

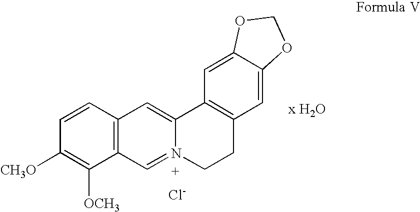

Additional ionized forms, including protoberberine forms and derivatives are generalized by the structure of formula VI wherein Y is a halide and exemplified in table 4 below.

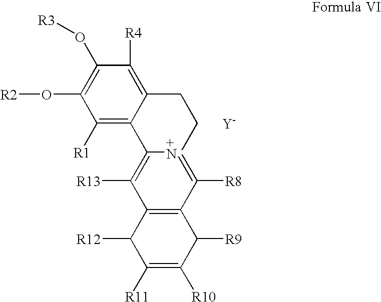

TABLE 4
Exemplary forms of protoberberine compounds and derivatives of formula VI.

| Composition | | | | | | | | | | | |
|---|---|---|---|---|---|---|---|---|---|---|---|
| No. | R¹ | R² | R³ | R⁴ | R⁸ | R⁹ | R¹⁰ | R¹¹ | R¹² | R¹³ | Y |
| 96 | H | —CH₂— | | H | H | CH₃O | CH₃O | H | H | CH₃ | I |
| 97 | H | H | H | H | H | CH₃O | CH₃O | H | H | CH₃ | Cl |
| 98 | H | H | H | H | H | OH | OH | H | H | H | Cl |
| 99 | H | H | H | H | H | OH | OH | H | H | CH₃ | Cl |
| 100 | H | C₂H₅ | C₂H₅ | H | H | C₂H₅O | C₂H₅O | H | H | C₂H₅ | Cl |
| 101 | H | —CH₂— | | H | H | CH₃O | CH₃O | H | H | C₂H₅ | I |
| 102 | H | H | H | H | H | OH | OH | H | H | C₂H₅ | Cl |
| 103 | H | CH₃ | CH₃ | H | H | CH₃O | CH₃O | H | H | C₂H₅ | Cl |
| 104 | H | —CH₂— | | H | H | CH₃O | CH₃O | H | H | Allyl | I |
| 105 | H | H | H | H | H | OH | OH | H | H | Allyl | Cl |
| 106 | H | CH₃ | CH₃ | H | H | CH₃O | CH₃O | H | H | n-propyl | I |
| 107 | H | CH₃ | CH₃ | H | H | CH₃O | CH₃O | H | H | n-Bu | I |
| 108 | H | CH₃ | CH₃ | H | H | CH₃O | CH₃O | H | H | 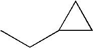 | I |
| 109 | H | CH₃ | CH₃ | H | H | CH₃O | CH₃O | H | H | n-Octyl | I |
| 110 | H | CH₃ | CH₃ | H | H | CH₃O | CH₃O | H | H | 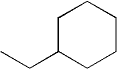 | I |
| 111 | H | CH₃ | CH₃ | H | H | CH₃O | CH₃O | H | H | 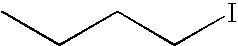 | I |
| 112 | H | n-Bu | n-Bu | H | H | n-Bu | n-Bu | H | H | H | Cl |
| 113 | H | CH₃ | CH₃ | H | H | CH₃O | CH₃O | H | H | 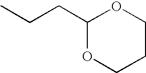 | Cl |
| 114 | H | CH₃ | CH₃ | H | H | CH₃O | CH₃O | H | H | 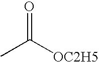 | Cl |
| 115 | H | CH₃ | CH₃ | H | H | CH₃O | CH₃O | H | H | 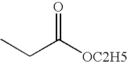 | Cl |
| 116 | H | CH₃ | CH₃ | H | H | CH₃O | CH₃O | H | H | 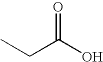 | Cl |
| 117 | H | —CH₂— | | H | H | CH₃O | CH₃O | H | H | 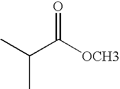 | Br |
| 118 | H | —CH₂— | | H | H | CH₃O | CH₃O | H | H | 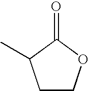 | Br |
| 119 | H | —CH₂— | | H | H | CH₃O | CH₃O | H | H | 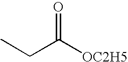 | Br |
| 120 | H | CH₃ | CH₃ | H | H | 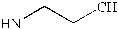 | CH₃O | H | H | C₂H₅ | Cl |

Additional discussion regarding exemplary forms of berberine compounds and derivatives contemplated for use within the methods and compositions is provided in U.S. Pat. No. 6,239,139, issued May 29, 2001; U.S. Pat. No. 6,030,979, issued Feb. 29, 2000; U.S. Pat. No. 6,028,196, issued Feb. 22, 2000, each of which is incorporated herein by reference.

While berberine, related berberine and proto-berberine compounds and derivative compounds may be generated by any methods known to those skilled in the art, exemplary compounds for use within the invention may also be generated, for example, according to Routes 1, 2, 3, and 4 described herein, below. These reaction and synthetic schemes are provided for illustrative purposes only, and it is understood that abbreviated, alternate, and modified schemes, e.g., encompassing essential elements of these schemes, or their equivalents, are also contemplated within the scope of the invention.

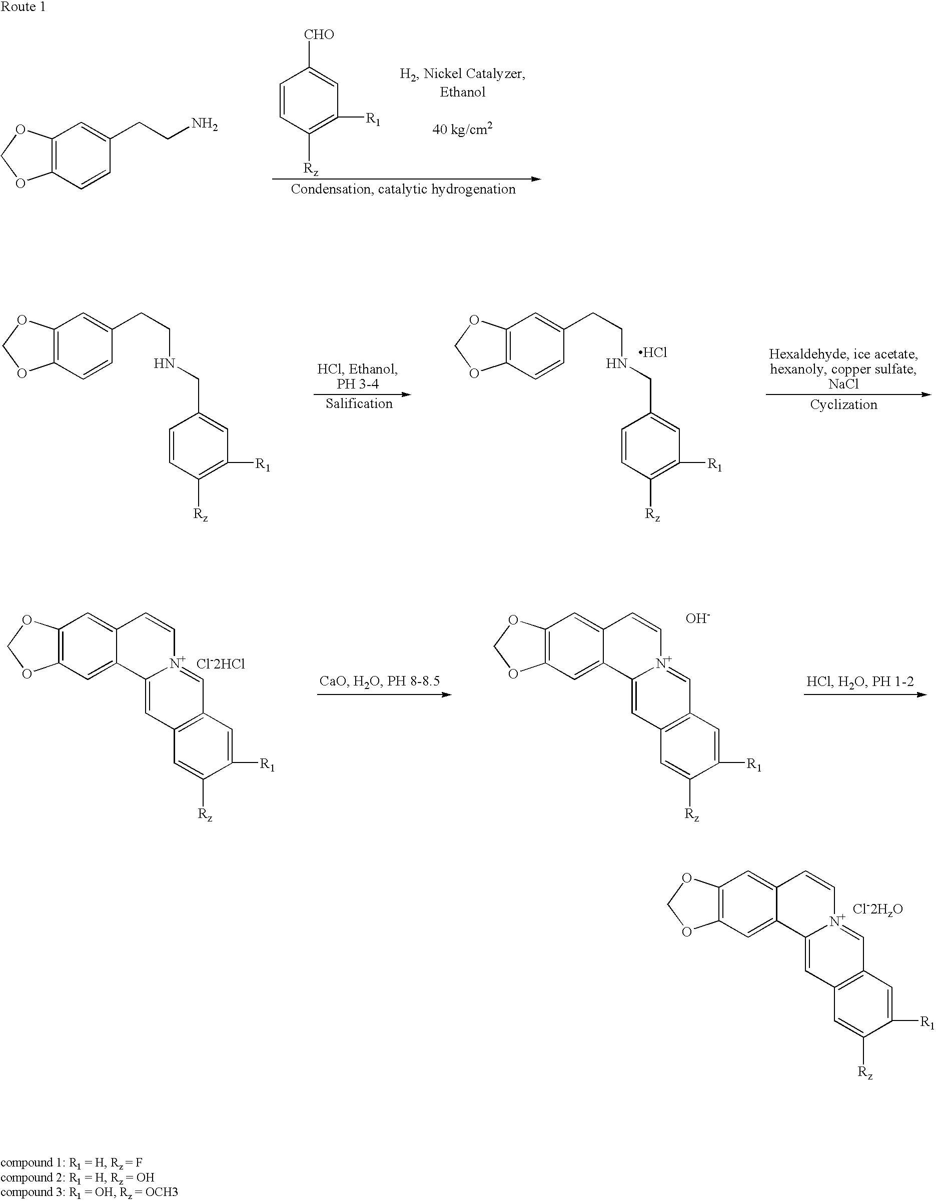

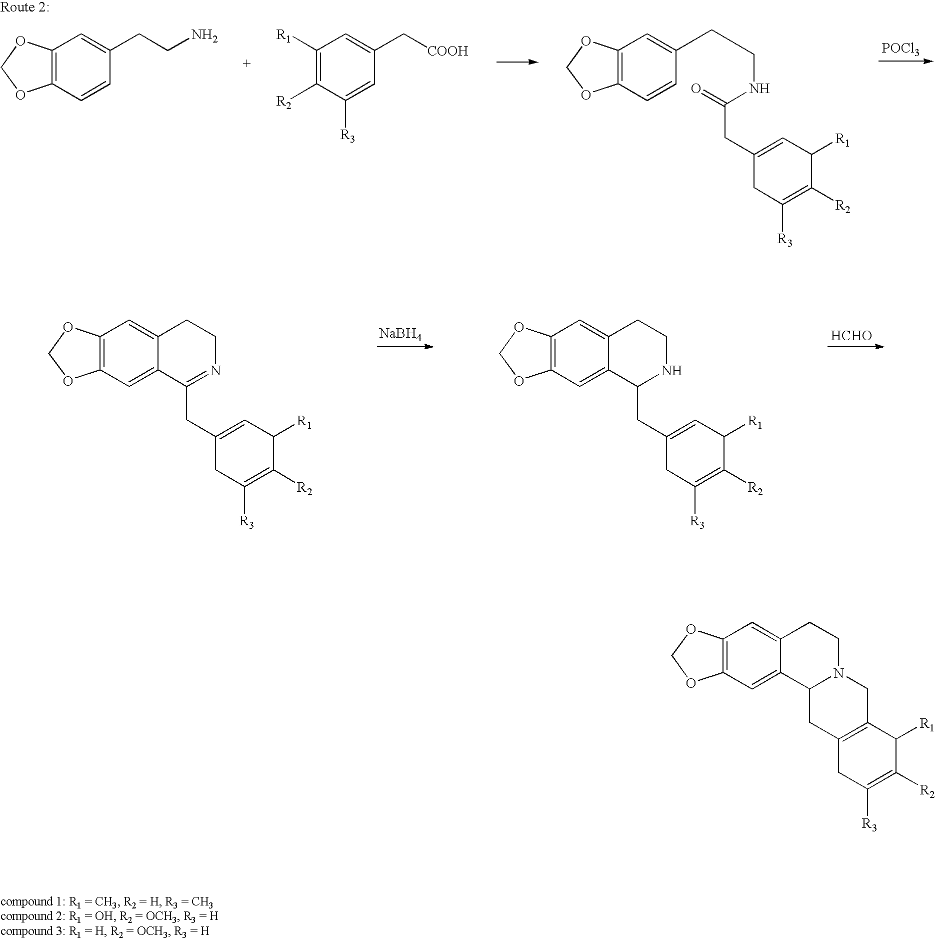

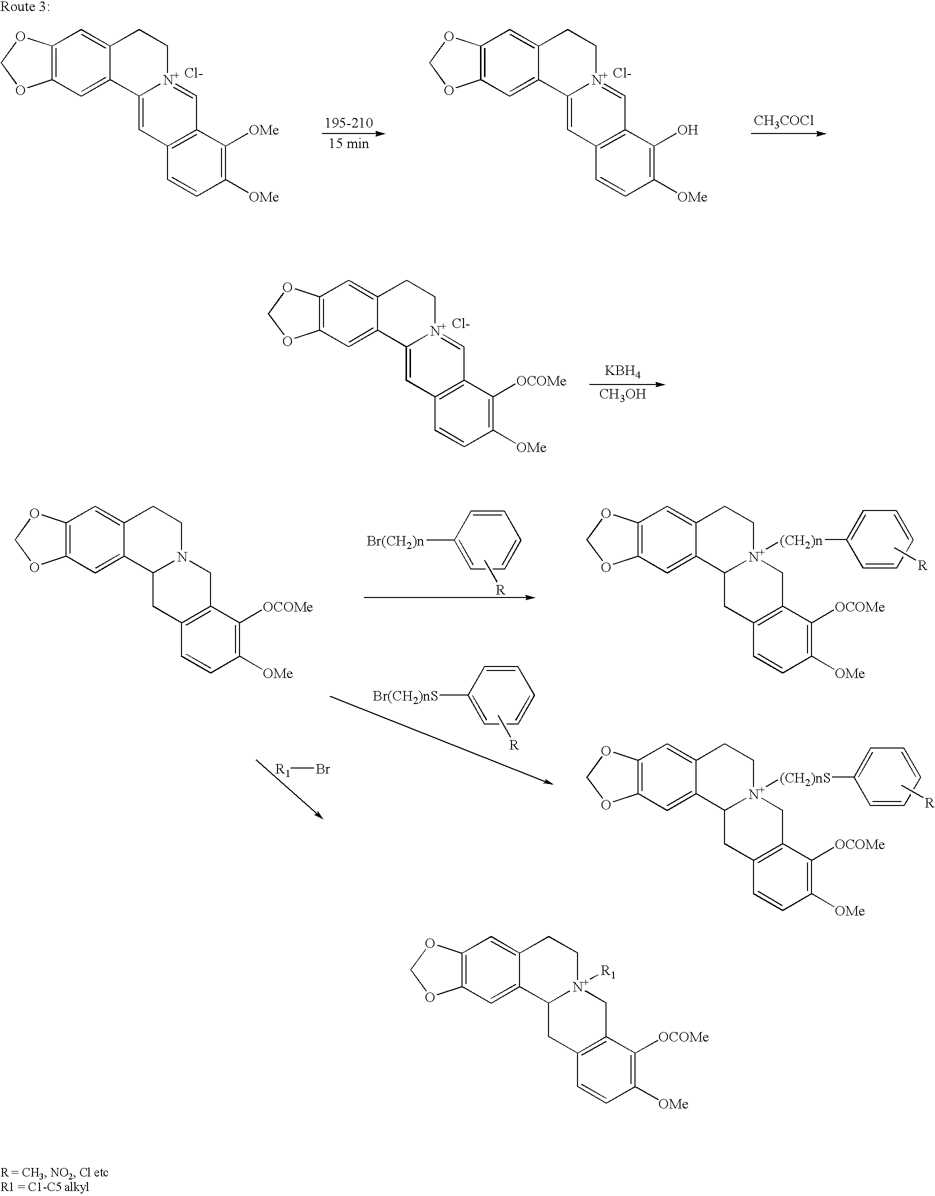

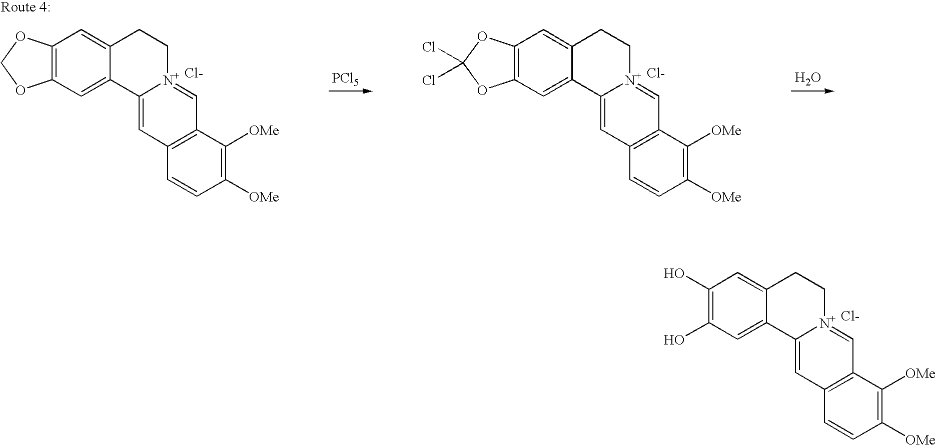

Lipid lowering compositions comprising a compound of formula I, including pharmaceutical formulations of the invention, comprise a lipid lowering effective amount of a berberine compound, berberine related, proto-berberine or derivative compound of Formula I, which is effective for prophylaxis and/or treatment of hyperlipidemia and elevated cholesterol in a mammalian subject. Typically, a lipid lowering effective amount, including a cholesterol lowering effective amount, of a berberine compound, berberine related, proto-berberine or derivative compound of Formula I will comprise an amount of the active compound which is therapeutically effective, in a single or multiple unit dosage form, over a specified period of therapeutic intervention, to measurably alleviate one or more symptoms of hyperlipidemia or elevated cholesterol in the subject, and/or to alleviate one or more symptom(s) of a cardiovascular disease or condition in the subject. Within exemplary embodiments, these compositions are effective within in vivo treatment methods to alleviate hyperlipidemia.

Lipid lowering compositions of the invention typically comprise a lipid lowering effective amount or unit dosage of a berberine compound, berberine related, proto-berberine or derivative compound of Formula I, which may be formulated with one or more pharmaceutically acceptable carriers, excipients, vehicles, emulsifiers, stabilizers, preservatives, buffers, and/or other additives that may enhance stability, delivery, absorption, half-life, efficacy, pharmacokinetics, and/or pharmacodynamics, reduce adverse side effects, or provide other advantages for pharmaceutical use. Lipid lowering effective amounts including cholesterol lowering effective amounts of a berberine compound, berberine related, proto-berberine or derivative compound (e.g., a unit dose comprising an effective concentration/amount of berberine, or of a selected pharmaceutically acceptable salt, isomer, enantiomer, solvate, polymorph and/or prodrug of berberine) will be readily determined by those of ordinary skill in the art, depending on clinical and patient-specific factors. Suitable effective unit dosage amounts of the active compounds for administration to mammalian subjects, including humans, may range from 10 to 1500 mg, 20 to 1000 mg, 25 to 750 mg, 50 to 500 mg, 150 to 500 mg, 100 to 200 mg, 200 to 400 mg, or 400 to 600 mg. In certain embodiments, the anti-hyperlipidemia or hypolipidemia effective dosage of a berberine compound, berberine related, proto-berberine or derivative compound of Formula I may be selected within narrower ranges of, for example, 10 to 25 mg, 30-50 mg, 75 to 100 mg, 100 to 250 mg, or 250 to 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In one exemplary embodiment, dosages of 10 to 25 mg, 30-50 mg, 75 to 100 mg, 100 to 250 mg, or 250 to 500 mg, are administered one, two, three, four, or five times per day. In more detailed embodiments, dosages of 50-75 mg, 100-200 mg, 250-400 mg, or 400-600 mg are administered once or twice daily. In alternate embodiments, dosages are calculated based on body weight, and may be administered, for example, in amounts from about 0.5 mg/kg to about 100 mg/kg per day, 1 mg/kg to about 75 mg/kg per day, 1 mg/kg to about 50 mg/kg per day, 2 mg/kg to about 50 mg/kg per day, 2 mg/kg to about 30 mg/kg per day or 3 mg/kg to about 30 mg/kg per day.

Glucose lowering compositions comprising a compound of formula I, including pharmaceutical formulations of the invention, comprise a glucose lowering effective amount of a berberine compound, berberine related, proto-berberine or derivative compound of Formula I, which is effective for prophylaxis and/or treatment of hyperglycemia in a mammalian subject. Typically, a glucose lowering effective amount, of a berberine compound, berberine related, proto-berberine or derivative compound of Formula I including a glycosylated derivative will comprise an amount of the active compound which is therapeutically effective, in a single or multiple unit dosage form, over a specified period of therapeutic intervention, to measurably alleviate one or more symptoms of hyperglycemia in the subject, and/or to alleviate one or more symptom(s) of a cardiovascular disease or condition in the subject. Within exemplary embodiments, these compositions are effective within in vivo treatment methods to alleviate hyperglycemia.

Insulin sensitivity increasing and insulin resistance decreasing compositions comprising a compound of formula I, including pharmaceutical formulations of the invention, comprise an insulin sensitivity increasing and/or insulin resistance decreasing effective amounts of a berberine compound, berberine related, proto-berberine or derivative compound of Formula I, which is effective for prophylaxis and/or treatment of insulin resistance in a mammalian subject. Typically, a insulin sensitivity increasing and/or insulin resistance decreasing effective amount, of a berberine compound, berberine related, proto-berberine or derivative compound of Formula I including a glycosylated derivative will comprise an amount of the active compound which is therapeutically effective, in a single or multiple unit dosage form, over a specified period of therapeutic intervention, to measurably alleviate one or more symptoms of insulin resistance in the subject, and/or to alleviate one or more symptom(s) of a cardiovascular disease or condition in the subject. Within exemplary embodiments, these compositions are effective within in vivo treatment methods to alleviate insulin resistance.

Glucose lowering or insulin sensitivity increasing/insulin resistance decreasing compositions of the invention typically comprise a glucose lowering effective amount or unit dosage of a berberine compound, berberine related, proto-berberine or derivative compound of Formula I, which may be formulated with one or more pharmaceutically acceptable carriers, excipients, vehicles, emulsifiers, stabilizers, preservatives, buffers, and/or other additives that may enhance stability, delivery, absorption, half-life, efficacy, pharmacokinetics, and/or pharmacodynamics, reduce adverse side effects, or provide other advantages for pharmaceutical use. Glucose lowering effective amounts of a berberine compound, berberine related, proto-berberine or derivative compound (e.g., a unit dose comprising an effective concentration/amount of berberine, or of a selected pharmaceutically acceptable salt, isomer, enantiomer, solvate, polymorph and/or prodrug of berberine) will be readily determined by those of ordinary skill in the art, depending on clinical and patient-specific factors. Suitable effective unit dosage amounts of the active compounds for administration to mammalian subjects, including humans, may range from 10 to 1500 mg, 20 to 1000 mg, 25 to 750 mg, 50 to 500 mg, or 150 to 500 mg. In certain embodiments, the anti-hyperglycemic effective dosage of a berberine compound, berberine related, proto-berberine or derivative compound of Formula I may be selected within narrower ranges of, for example, 10 to 25 mg, 30-50 mg, 75 to 100 mg, 100 to 250 mg, or 250 to 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In one exemplary embodiment, dosages of 10 to 25 mg, 30-50 mg, 75 to 100 mg, 100 to 250 mg, or 250 to 500 mg, are administered one, two, three, four, or five times per day. In more detailed embodiments, dosages of 50-75 mg, 100-200 mg, 250-400 mg, or 400-600 mg are administered once or twice daily. In alternate embodiments, dosages are calculated based on body weight, and may be administered, for example, in amounts from about 0.5 mg/kg to about 100 mg/kg per day, 1 mg/kg to about 75 mg/kg per day, 1 mg/kg to about 50 mg/kg per day, 2 mg/kg to about 50 mg/kg per day, 2 mg/kg to about 30 mg/kg per day or 3 mg/kg to about 30 mg/kg per day.

The amount, timing and mode of delivery of compositions of the invention comprising an anti-hyperlipidemia and/or anti-hyperglycemic effective amount of a berberine compound, berberine related, proto-berberine or derivative compound of Formula I will be routinely adjusted on an individual basis, depending on such factors as weight, age, gender, and condition of the individual, the acuteness of the hyperlipidemia and/or related symptoms, whether the administration is prophylactic or therapeutic, and on the basis of other factors known to effect drug delivery, absorption, pharmacokinetics, including half-life, and efficacy.

An effective dose or multi-dose treatment regimen for the instant lipid lowering formulations will ordinarily be selected to approximate a minimal dosing regimen that is necessary and sufficient to substantially prevent or alleviate hyperlipidemia and cardiovascular diseases in the subject, and/or to substantially prevent or alleviate one or more symptoms associated with hyperlipidemia in the subject. A dosage and administration protocol will often include repeated dosing therapy over a course of several days or even one or more weeks or years. An effective treatment regime may also involve prophylactic dosage administered on a day or multi-dose per day basis lasting over the course of days, weeks, months or even years.

An effective dose or multi-dose treatment regimen for the instant glucose lowering formulations will ordinarily be selected to approximate a minimal dosing regimen that is necessary and sufficient to substantially prevent or alleviate hyperglycemia in the subject, and/or to substantially prevent or alleviate one or more symptoms associated with hyperglycemia in the subject. A dosage and administration protocol will often include repeated dosing therapy over a course of several days or even one or more weeks or years. An effective treatment regime may also involve prophylactic dosage administered on a day or multi-dose per day basis lasting over the course of days, weeks, months or even years.

Various assays and model systems can be readily employed to determine the therapeutic effectiveness of anti-hyperlipidemia treatment according to the invention. For example, blood tests to measure total cholesterol as well as triglycerides, LDL and HDL levels are routinely given. Individuals with a total cholesterol level of greater than 200 mg/dL are considered borderline high risk for cardiovascular disease. Those with a total cholesterol level greater than 239 mg/dL are considered to be at high risk. An LDL level of less than 100 mg/dL is considered optimal. LDL levels between 130 to 159 mg/dL are borderline high risk. LDL levels between 160 to 189 mg/dL are at high risk for cardiovascular disease and those individuals with an LDL greater than 190 mg/dL are considered to be at very high risk for cardiovascular disease. Triglyceride levels of less than 150 mg/dL are considered normal. Levels between 150-199 mg/dL are borderline high and levels above 200 are considered to put the individual at high risk for cardiovascular disease. Lipid levels can be determined by standard blood lipid profile tests. Effective amounts of the compositions of the invention will lower elevated lipid levels by at least 10%, 20%, 30%, 50% or greater reduction, up to a 75-90%, or 95% or greater. Effective amounts will also move the lipid profile of an individual towards the optimal category for each lipid, i.e., decrease LDL levels from 190 mg/dl to within 130 to 159 mg/dL or even further to below 100 mg/dL. Effective amounts may further decrease LDL or triglyceride levels by about 10 to about 70 mg/dL, by about 20 to about 50 mg/dL, by about 20 to about 30 mg/dL, or by about 10 to about 20 mg/dL.

Individuals may also be evaluated using a hs-CRP (high-sensitivity C-reactive protein) blood test. Those with a hs-CRP result of less than 1.0 mg/L are at low risk for cardiovascular disease. Individuals with a hs-CRP result between about 1.0 to 3.0 mg/L are at average risk for cardiovascular disease. Those with a hs-CRP result greater than 3.0 mg/L are at high risk of cardiovascular disease. Effective amounts of the compositions of the present invention will lower hs-CRP results below 3.0 mg/L. Effective amounts of the compositions of the present invention can lower hs-CRP results by about 0.5 to about 3.0 mg/L, and further by about 0.5 to about 2.0 mg/L. An effective amount of a berberine compound of the present invention will lower the hs-CRP level from over 3.0 mg/L to between 1.0 and 3.0 mg/l, more preferably to about 1.0 mg/L to about 0.6 mg/L.

Therapeutic effectiveness may be determined, for example, through a change in body fat as determined by body fat measurements. Body fat measurements may be determined by a variety of means including, but not limited to, determinations of skinfold thickness, bioelectrical impedance, air displacement plethysmography, underwater weighing, DEXA scans, measurement on a scale or calculation of body mass index (BMI).

Percentages of weight due to body fat for normal men are between 10-20%. In athletes, the normal range is between 6-10%. In women, the normal range is between 15-25% and in athletic women it is between 10-15%. Effective amounts of the compounds of the present invention will decrease body fat percentages from above 20-25%. Effective amounts may also decrease body fat percentages to within the normal ranges for that individual. Effectiveness may also be demonstrated by a 2-50%, 10-40%, 15-30%, 20-25% decrease in body fat.

Skinfold measurements measure subcutaneous fat located directly beneath the skin by grasping a fold of skin and subcutaneous fat between the thumb and forefinger and pulling it away from the underlying muscle tissue. The thickness of the double layer of skin and subcutaneous tissue is then read with a caliper. The five most frequently measured sites are the upper arm, below the scapula, above the hip bone, the abdomen, and the thigh. Skinfold measurements are used to determine relative fatness, changes in physical conditioning programs, and the percentage of body fat in desirable body weight. Effective amounts of berberine compounds will decrease body fat percentages by 2-50%, 10-40%, 15-30%, 20-25%, 30-40% or more.

Body fat percentages can also be determined by body impedance measurements. Body impedance is measured when a small electrical signal is passed through the body carried by water and fluids. Impedance is greatest in fat tissue, which contains only 10-20% water, while fat-free mass, which contains 70-75% water, allows the signal to pass much more easily. By using the impedance measurements along with a person's height, weight, and body type (gender, age, fitness level), it is possible to calculate the percentage of body fat, fat-free mass, hydration level, and other body composition values. Effective amounts of berberine compounds will decrease body fat percentages by 2-50%, 10-40%, 15-30%, 20-25%, 30-40% or more.

Hydrostatic or underwater weighing is another method for determining lean muscle mass and body fat percentages. It is based upon the application of the Archimedes principle, and requires weighing the subject on land, repeated weighing under water, and an estimation of air present in the lungs of the subject using gas dilution techniques. To perform the analysis, an individual is weighed as normal. The subject, in minimal clothing, then sits on a special seat, expels all air from the lungs and is lowered into a tank until all body parts are emerged. Underwater weight is then determined. Body density is then determined using the following calculation: Body density=Wa/(((Wa−Ww)/Dw)−(RV+100 cc)), where Wa=body weight in air (kg), Ww=body weight in water (kg), Dw=density of water, RV=residual lung volume, and 100 cc is the correction for air trapped in the gastrointestinal tract.

DEXA, or dual energy x-ray absorptiometry scans determine whole body as well as regional measurements of bone mass, lean mass, and fat mass. Total fat mass is expressed in kg and as a percentage of body mass. These are calculated by integrating the measurements for the whole body and different automatic default regions such as arms, trunk, and legs.

Body fat percentages may further be determined by air displacement plethysmography. Air displacement plethysmography determines the volume of a subject to be measured by measuring the volume of air displaced by the subject in an enclosed chamber. The volume of air in the chamber is calculated through application of Boyle's Law and/or Poisson's Law to conditions within the chamber. More particularly, in the most prevalent method of air displacement plethysmography used for measuring human body composition (such as disclosed in U.S. Pat. No. 4,369,652, issued to Gundlach, and U.S. Pat. No. 5,105,825, issued to Dempster), volume perturbations of a fixed frequency of oscillation are induced within a measurement chamber, which perturbations lead to pressure fluctuations within the chamber. The amplitude of the pressure fluctuations is determined and used to calculate the volume of air within the chamber using Boyle's Law (defining the relationship of pressure and volume under isothermal conditions) or Poisson's law (defining the relationship of pressure and volume under adiabatic conditions). Body volume is then calculated indirectly by subtracting the volume of air remaining inside the chamber when the subject is inside from the volume of air in the chamber when it is empty. Once the volume of the subject is known, body composition can be calculated based on the measured subject volume, weight of the subject, and subject surface area (which, for human subjects, is a function of subject weight and subject height), using known formulas defining the relationship between density and human fat mass.

Therapeutic effectiveness of berberine treatment according to the invention may further be demonstrated, for example, through a change in body mass index. Body Mass Index (BMI) has been recognized by the U.S. Department of Health as a reference relationship between a person's height and weight and can be used to determine when extra weight above an average or normal weight range for a person of a given height can translate into and signal increased probability for additional health risks for that person. While BMI does not directly measure percent of body fat, higher BMIs are usually associated with an increase in body fat, and thus excess weight. A desired BMI range is from about 18 kg/m² to about 24 kg/m², wherein a person is considered to have a healthful weight for the person's height and is neither overweight nor underweight. A person with a BMI above 24 kg/m², such as from about 25 kg/m² to about 30 kg/m², is considered to be overweight, and a person with a BMI above about 30 kg/m² is considered to be obese. A person with a BMI above about 40 kg/m² is considered to be morbidly obese. In another aspect, an individual who has a BMI in the range of about 25 kg/m² to about 35 kg/m², and has a waist size of over 40 inches for a man and over 35 inches for a woman, is considered to be at especially high risk for health problems. Effectiveness of berberine compounds may be demonstrated by a reduction in the body mass index from a range between 40 kg/m² to about 30 kg/m² to 25 kg/m² to about 24 kg/m². A compound of the present invention may also reduce BMI from a range above 30 kg/m² to a range between 30 kg/m² to 25 kg/m² and more preferably to about 24 kg/m². Effectiveness may further be demonstrated by a decrease in body weight from 1-25%, 3-15%, 2-50%, 10-40%, 15-30%, 20-25%. Effectiveness may additionally be demonstrated by a decrease in BMI by 2-50%, 10-40%, 15-30%, 20-25%, 30-40% or more. Effective amounts of berberine compounds will lower an individual's BMI to within about 18 kg/m² to about 24 kg/m².

Therapeutic effectiveness of berberine compounds of the present invention may also be determined by changes in the waist/hip ratio. The waist/hip ratio is determined by dividing the circumference of the waist by the circumference of the hip. Women should have a waist/hip ratio of 0.8 or less and men should have a waist/hip ratio of 0.95 or less. Effective amounts of berberine compounds will lower the waist/hip ratio by about 2-50%, 10-40%, 15-30%, 20-25% or more. The waist/hip ratio of a female subject may be lowered to 0.8 or less and the ratio of a male subject to a ratio of 0.95 or less.

Therapeutic effectiveness of berberine compounds of the present invention may also be determined by a decrease in weight of the subject as determined by a standard scale. Effective amounts of berberine compounds will decrease weight by about 2-50%, 10-40%, 15-30%, 20-25% or more.

Therapeutic effectiveness of berberine compounds of the present invention may also be determined by a decrease in waist circumference. The waist circumference of men will decrease from more than 40 inches and the waist circumference of women will decrease from more than 35 inches. Effective amounts of berberine compounds of the present invention will decrease waist circumference by about 2-50%, 10-40%, 15-30%, 20-25% or more.

Therapeutic effectiveness may also be demonstrated with a decrease in fasting glucose. A fasting glucose test measures blood glucose after an overnight fast. Fasting glucose levels of 100 to 125 mg/dL are above normal. Effective amounts of berberine compounds of the present invention will decrease fasting glucose levels by about 2-50%, 10-40%, 15-30%, 20-25% or more. An effective amount of a berberine compound will lower the fasting glucose level from above 125 mg/dL to a range between 70 to 99 mg/dL. An effective amount of a composition of the present invention may further lower fasting glucose levels by about 1 to about 5 mg/dL, by about 1 to about 10 mg/dL, by about 5 to about 20 mg/dL, by about 5 to about 30 mg/dL, by about 20 to about 60 mg/dL or more.

Therapeutic effectiveness may further be demonstrated by a glucose tolerance test. A glucose tolerance test is taken after an overnight fast and 2 hours after consumption of a glucose solution. An effective amount of a berberine compound of the present invention will lower glucose levels by about 2-50%, 10-40%, 15-30%, 20-25% or more. Glucose levels may be lowered from above 200 mg/dL to a range between 140 to 200 mg/dL, and more preferably to below 140 mg/dL. An effective amount of a berberine compound of the present invention may lower glucose levels by about 1 to about 5 mg/dL, by about 1 to about 10 mg/dL, by about 5 to about 20 mg/dL, by about 5 to about 30 mg/dL, by about 20 to about 60 mg/dL or more.

Therapeutic effectiveness may additionally be demonstrated by a hyperinsulinemic euglycemic clamp study. In a hyperinsulinemic euglycemic clamp study, insulin and glucose are infused intravenously at several different doses to determine what levels of insulin control different levels of glucose. Through a peripheral vein, insulin is infused at 0.06 units per kg body weight per minute. In order to compensate for the insulin infusion, glucose 20% is infused to maintain blood sugar levels between 5 and 5.5 mmol/l. The rate of glucose infusion is determined by checking the blood sugar levels every 5 minutes. The rate of glucose infusion during the last 30 minutes of the test determines insulin sensitivity. If high levels (7.5 mg/min or higher) are required, the patient is insulin-sensitive. Very low levels (4.0 mg/min or lower) indicate that the body is resistant to insulin action. Levels between 4.0 and 7.5 mg/min are not definitive and suggest “impaired glucose tolerance,” an early sign of insulin resistance. Effective amounts of berberine compounds of the present invention will increase the amount of glucose consumed from below 4.0 mg/min to about 7.5 mg/min or more. Effective amounts of berberine will increase glucose clearance by 2-50%, 10-40%, 15-30%, 20-25% or more.

Therapeutic effectiveness may further be determined by a glycohemoglobin test. A glycohemoglobin test is a blood test that measures the amount of glucose bound to hemoglobin. The results reflect the amount of glycohemoglobin divided by the total amount of hemoglobin multiplied by 100 (to produce a percentage). An effective amount of a berberine compound of the present invention will decrease the hemoglobin A1c % to less than 14%, preferably to between 8 and 10%, more preferably to between 5 and 8%, more preferably to between 6 and 8% and most preferably to between 4 to 6%. Berberine may decrease the hemoglobin A1c % by about 2-50%, 10-40%, 15-30%, 20-25% or more.

Therapeutic effectiveness may also be calculated through an insulin suppression test. During this test, subjects receive a continuous infusion of a fixed combination of glucose and insulin, while endogenous insulin secretion is blocked by somatostatin or octreotide. After around 150 min, a steady-state of glucose and insulin levels is achieved, at which time, the steady-state glucose and insulin levels are measured and their quotient calculated as a measure of insulin sensitivity. An effective amount of berberine compounds of the present invention will increase insulin sensitivity by about 2-50%, 10-40%, 15-30%, 20-25% or more.

Therapeutic effectiveness may additionally be determined by the C13 glucose breath test in which glucose labeled with non-radioactive C13, is ingested and the byproduct of its metabolism ¹³CO₂ is detected in expired air. In insulin resistant states glucose uptake would be impaired and the production of ¹³CO₂ would therefore also be impaired. An effective amount of a berberine compound of the present invention will increase ¹³CO₂ by about 2-50%, 10-40%, 15-30%, 20-25% or more.

Therapeutic effectiveness may further be determined by a random plasma glucose test. An effective amount of a berberine compound of the present invention will decrease blood glucose from above 200 mg/dL to a range between 140 to 200 mg/dL, and more preferably to below 140 mg/dL.

In another aspect of the invention, therapeutic effectiveness may be determined by a CIGMA test in which 180 mg/min⁻¹/m⁻² of glucose is infused for 120 min at a rate of 5 mg/kg with blood samples are taken at 110, 115 and 120 minutes. An effective amount of berberine, a berberine compound, a proto-berberine compound or derivative will increase glucose clearance by 2-50%, 10-40%, 15-30%, 20-25% or more.

In a further aspect of the invention, therapeutic effectiveness may be determined by a FSIVGTT test in which an intravenous glucose bolus (0.3 g/kg) is administered followed by a 5 minute insulin infusion 20 minutes later. Blood samples are tested for glucose every two minutes for the first 20 minutes and samples are tested for glucose and insulin levels at 22, 24, 26, 28, 30, 33, 36, 40, 50, 60, 70, 80, 100, 120, 140, 160 and 180 minutes. An effective amount of a berberine compound, a proto-berberine compound or derivative will increase glucose clearance by 2-50%, 10-40%, 15-30%, 20-25% or more.

Effectiveness of berberine compounds may further be determined by blood pressure testing. Effective amounts of berberine compounds of the present invention will lower blood pressure from above 150/100 mm Hg to less than 120/80 mm Hg, preferably to between about 139/89 mm to about 120/80 mm Hg, most preferably to less than 120/80 mm Hg. Preferably the methods and compositions of the present invention will lower blood pressure to between 110/60 mm Hg to about 120/70 mmHg.

Therapeutic effectiveness of berberine compounds of the present invention may also be determined by a D-dimer test. An effective amount of berberine will decrease the amount of d-dimer in a sample to about 0-300 ng/ml. An effective amount of a berberine compound of the present invention may also decrease the d-dimer level in a sample by about 2-50%, 10-40%, 15-30%, 20-25% or more.

Within additional aspects of the invention, effectiveness of the compositions and methods of the invention may also be demonstrated by a decrease or improvement in the complications or symptoms of metabolic and cardiovascular disorders including fatty liver, reproductive abnormalities, growth abnormalities, arterial plaque accumulation, osteoarthritis, gout, joint pain, respiratory problems, skin conditions, sleep apnea, idiopathic intracranial hypertension, lower extremity venous stasis disease, gastro-esophageal reflux, urinary stress incontinence, kidney damage, cardiovascular diseases such as atherosclerosis, coronary artery disease, enlarged heart, diabetic cardiomyopathy, angina pectoris, carotid artery disease, peripheral vascular disease, stroke, cerebral arteriosclerosis, myocardial infarction, cerebral infarction, restenosis following balloon angioplasty, intermittent claudication, dyslipidemia post-prandial lipidemia, high blood pressure and xanthoma.

For each of the indicated conditions described herein, test subjects will exhibit a 5%, 10%, 20%, 30%, 50% or greater reduction, up to a 75-90%, or 95% or greater, reduction, in one or more symptom(s) caused by, or associated with, hyperlipidemia, hyperglycemia, elevated cholesterol, hypertension, metabolic syndrome, obesity, diabetes, elevated glucose and/or a targeted cardiovascular disease or condition in the subject, compared to placebo-treated or other suitable control subjects.

Within additional aspects of the invention, combinatorial lipid lowering formulations and coordinate administration methods are provided which employ an effective amount of a berberine compound, berberine related, proto-berberine or derivative compound of Formula I and one or more secondary or adjunctive agent(s) that is/are combinatorially formulated or coordinately administered with the berberine compound or berberine related or derivative compound to yield a combined, multi-active agent anti-hyperlipidemia composition or coordinate treatment method. Exemplary combinatorial formulations and coordinate treatment methods in this context employ the berberine compound, berberine related, proto-berberine or derivative compound in combination with the one or more secondary anti-hyperlipidemia agent(s), or with one or more adjunctive therapeutic agent(s) that is/are useful for treatment or prophylaxis of the targeted (or associated) disease, condition and/or symptom(s) in the selected combinatorial formulation or coordinate treatment regimen.

Within additional aspects of the invention, combinatorial glucose lowering formulations and coordinate administration methods are provided which employ an effective amount of a berberine compound, berberine related, proto-berberine or derivative compound of Formula I and one or more secondary or adjunctive agent(s) that is/are combinatorially formulated or coordinately administered with the berberine compound, berberine related, proto-berberine or derivative compound to yield a combined, multi-active agent anti-hyperglycemic composition or coordinate treatment method. Exemplary combinatorial formulations and coordinate treatment methods in this context employ the berberine compound, berberine related, proto-berberine or derivative compound in combination with the one or more secondary anti-hyperglycemic agent(s), or with one or more adjunctive therapeutic agent(s) that is/are useful for treatment or prophylaxis of the targeted (or associated) disease, condition and/or symptom(s) in the selected combinatorial formulation or coordinate treatment regimen.

Within further aspects of the invention, combinatorial hypertension lowering formulations and coordinate administration methods are provided which employ an effective amount of a berberine compound, berberine related, proto-berberine or derivative compound of Formula I and one or more secondary or adjunctive agent(s) that is/are combinatorially formulated or coordinately administered with the berberine compound or berberine related or derivative compound to yield a combined, multi-active agent anti-hypertensive composition or coordinate treatment method. Exemplary combinatorial formulations and coordinate treatment methods in this context employ the berberine compound, berberine related, proto-berberine or derivative compound in combination with the one or more secondary anti-hypertensive agent(s), or with one or more adjunctive therapeutic agent(s) that is/are useful for treatment or prophylaxis of the targeted (or associated) disease, condition and/or symptom(s) in the selected combinatorial formulation or coordinate treatment regimen

For most combinatorial formulations and coordinate treatment methods of the invention, a berberine compound, berberine related, proto-berberine or derivative compound of Formula I is formulated, or coordinately administered, in combination with one or more secondary or adjunctive therapeutic agent(s), to yield a combined formulation or coordinate treatment method that is combinatorially effective or coordinately useful to treat hyperlipidemia, hyperglycemia, hypertension, metabolic syndrome, diabetes, obesity, insulin resistance and/or one or more symptom(s) of a metabolic disorder or condition in the subject. Exemplary combinatorial formulations and coordinate treatment methods in this context employ a berberine compound, berberine related, proto-berberine or derivative compound of Formula I in combination with one or more secondary or adjunctive therapeutic agents selected from, e.g., The secondary or adjunctive therapeutic agents used in combination with, e.g., berberine in these embodiments may possess direct or indirect lipid and/or glucose lowering activity and/or hypertension decreasing activity, including cholesterol lowering activity, insulin resistance decreasing activity, insulin sensitivity increasing activity or glucose regulating activity, alone or in combination with, e.g., berberine, or may exhibit other useful adjunctive therapeutic activity in combination with, e.g., berberine. Useful adjunctive therapeutic agents in these combinatorial formulations and coordinate treatment methods include, for example, anti-hyperlipidemic agents; anti-dyslipidemic agents; plasma HDL-raising agents; anti-hypercholesterolemic agents, including, but not limited to, cholesterol-uptake inhibitors; cholesterol biosynthesis inhibitors, e.g., HMG-CoA reductase inhibitors (also referred to as statins, such as lovastatin, simvastatin, pravastatin, fluvastatin, rosuvastatin, pitavastatin, and atorvastatin); HMG-CoA synthase inhibitors; squalene epoxidase inhibitors or squalene synthetase inhibitors (also known as squalene synthase inhibitors); acyl-coenzyme A cholesterol acyltransferase (ACAT) inhibitors, including, but not limited to, melinamide; probucol; nicotinic acid and the salts thereof; niacinamide; cholesterol absorption inhibitors, including, but not limited to, P-sitosterol or ezetimibe; bile acid sequestrant anion exchange resins, including, but not limited to cholestyramine, colestipol, colesevelam or dialkylaminoalkyl derivatives of a cross-linked dextran; LDL receptor inducers; fibrates, including, but not limited to, clofibrate, bezafibrate, fenofibrate and gemfibrozil; vitamin B6 (also known as pyridoxine) and the pharmaceutically acceptable salts thereof, such as the HCl salt; vitamin B12 (also known as cyanocobalamin); vitamin B3 (also known as nicotinic acid and niacinamide, supra); anti-oxidant vitamins, including, but not limited to, vitamin C and E and betacarotene; angiotensin II receptor (AT₁) antagonist; renin inhibitors; platelet aggregation inhibitors, including, but not limited to, fibrinogen receptor antagonists, i.e., glycoprotein IIb/IIIa fibrinogen receptor antagonists; hormones, including but not limited to, estrogen; insulin; ion exchange resins; omega-3 oils; benfluorex; ethyl icosapentate; and amlodipine; appetite-suppressing agents or anti-obesity agents including, but not limited to, insulin sensitizers, protein tyrosine phosphatase-1B (PTP-1B) inhibitors, dipeptidyl peptidase IV (DP-IV) inhibitors, insulin or insulin mimetics, sequestrants, nicotinyl alcohol, nicotinic acid, PPARα agonists, PPAR γ agonists including glitazones, PPARα/γ dual agonists, inhibitors of cholesterol absorption, acyl CoA:cholesterol acyltransferase inhibitors, anti-oxidants, anti-obesity compounds, neuropeptide Y5 inhibitors, β₃ adrenergic receptor agonists, ileal bile acid transporter inhibitors, anti-inflammatories and cyclo-oxygenase 2 selective inhibitors; insulin; sulfonylureas, including but not limited to chlorpropamide, glipizide, glyburide, and glimepiride; cannabinoid antagonists including, but not limited to, rimonabant; camptothecin and camptothecin derivatives, DPP-4 blockers; biguanides, including but not limited to metformin and phenformin; thiazolidinediones including but not limited to rosiglitazone, troglitazone and pioglitazone; alpha-glucosidase inhibitors, including, but not limited to, acarbose and meglitol; D-phenylalanine derivatives; meglitinides; diuretics including, but not limited to, methyclothiazide, hydroflumethiazide, metolazone, chlorothiazide, methyclothiazide, hydrochlorothiazide, quinethazone, chlorthalidone, trichlormethiazide, bendroflumethiazide, polythiazide, hydroflumethiazide, spironolactone, triamterene, amiloride, bumetanide, torsemide, ethacrynic acid, furosemide; beta-blockers including, but not limited to acebutolol, atenolol, betaxolol, bisoprolol, carteolol, metoprolol, nadolol, pindolol, propranolol, and timolol.; angiotensin-converting enzyme (ACE) inhibitors including, but not limited to, benazepril, captopril; enalapril, fosinopril, lisinopril, moexipril, perindopril, quinapril, ramipril, and trandolapril; calcium channel blockers including, but not limited to, amlodipine, diltiazem, felodipine, isradipine, nicardipine sr, nifedipine er, nisoldipine, and verapamil; vasodilators including, but not limited to, nitric oxide, hydralazine, and prostacyclin; angiotensin II receptor blockers including, but not limited to, andesartan, eprosartan, irbesartan, losartan, olmesartan, telmisartan, and valsartan; alpha blockers including, but not limited to, doxazosin, prazosin and terazosin; alpha 2 agonists including, but not limited to clonidine and guanfacine. Such agents may be referred to in whole or in part as metabolic disorder therapeutics, metabolic syndrome therapeutics, anti-obesity therapeutics, anti-hypercholesterolemia therapeutics, anti-diabetic therapeutics, insulin resistance therapeutic agents, anti-hyperglycemia agents, insulin sensitivity increasing agents, anti-hypertensive agents, and/or blood glucose lowering therapeutic agents. Adjunctive therapies may also be used including, but not limited, physical treatments such as changes in diet, psychological counseling, behavior modification, exercise and surgery including, but not limited to, gastric partitioning procedures, jejunoileal bypass, stomach stapling, gastric bands, vertical banded gastroplasty, laparoscopic gastric banding, roux-en-Y gastric bypass, biliopancreatic bypass procedures and vagotomy. Some herbal remedies may also be employed effectively in combinatorial formulations and coordinate therapies for treating metabolic disorders, for example curcumin, gugulipid, garlic, vitamin E, soy, soluble fiber, fish oil, green tea, carnitine, chromium, coenzyme Q10, anti-oxidant vitamins, grape seed extract, pantothine, red yeast rice, and royal jelly.

In certain embodiments the invention provides combinatorial lipid lowering formulations comprising berberine and one or more adjunctive agent(s) having anti-inflammatory or lipid lowering activity. Within such combinatorial formulations, berberine and the adjunctive agent(s) having lipid lowering activity will be present in a combined formulation in lipid lowering effective amounts, alone or in combination. In exemplary embodiments, berberine and a non-berberine lipid lowering agent(s) will each be present in a lipid lowering amount (i.e., in singular dosage which will alone elicit a detectable anti-hyperlipidemia response in the subject). Alternatively, the combinatorial formulation may comprise one or both of the berberine and non-berberine agents in sub-therapeutic singular dosage amount(s), wherein the combinatorial formulation comprising both agents features a combined dosage of both agents that is collectively effective in eliciting a lipid lowering response. Thus, one or both of the berberine and non-berberine agents may be present in the formulation, or administered in a coordinate administration protocol, at a sub-therapeutic dose, but collectively in the formulation or method they elicit a detectable lipid lowering response in the subject.

In certain embodiments the invention provides combinatorial glucose lowering formulations comprising berberine and one or more adjunctive agent(s) having anti-inflammatory or glucose lowering activity. Within such combinatorial formulations, berberine and the adjunctive agent(s) having glucose lowering activity will be present in a combined formulation in glucose lowering effective amounts, alone or in combination. In exemplary embodiments, berberine and a non-berberine glucose lowering agent(s) will each be present in a glucose lowering amount (i.e., in singular dosage which will alone elicit a detectable anti-hyperlipidemia response in the subject). Alternatively, the combinatorial formulation may comprise one or both of the berberine and non-berberine agents in sub-therapeutic singular dosage amount(s), wherein the combinatorial formulation comprising both agents features a combined dosage of both agents that is collectively effective in eliciting a glucose lowering response. Thus, one or both of the berberine and non-berberine agents may be present in the formulation, or administered in a coordinate administration protocol, at a sub-therapeutic dose, but collectively in the formulation or method they elicit a detectable glucose lowering response in the subject.

In certain embodiments the invention provides combinatorial anti-hypertensive formulations comprising berberine and one or more adjunctive agent(s) having anti-inflammatory or anti-hypertensive activity. Within such combinatorial formulations, berberine and the adjunctive agent(s) having anti-hypertensive activity will be present in a combined formulation in hypertension lowering effective amounts, alone or in combination. In exemplary embodiments, berberine and a non-berberine anti-hypertensive agent(s) will each be present in a hypertension lowering amount (i.e., in singular dosage which will alone elicit a detectable anti-hyperlipidemia response in the subject). Alternatively, the combinatorial formulation may comprise one or both of the berberine and non-berberine agents in sub-therapeutic singular dosage amount(s), wherein the combinatorial formulation comprising both agents features a combined dosage of both agents that is collectively effective in eliciting a lipid lowering response. Thus, one or both of the berberine and non-berberine agents may be present in the formulation, or administered in a coordinate administration protocol, at a sub-therapeutic dose, but collectively in the formulation or method they elicit a detectable hypertension lowering response in the subject.

To practice coordinate administration methods of the invention, a berberine compound, berberine related, proto-berberine or derivative compound of Formula I may be administered, simultaneously or sequentially, in a coordinate treatment protocol with one or more of the secondary or adjunctive therapeutic agents contemplated herein. Thus, in certain embodiments a berberine compound, berberine related, proto-berberine or derivative compound is administered coordinately with a non-berberine lipid lowering agent; a non-berberine glucose lowering agent; a non-berberine insulin sensitivity increasing agent; a non-berberine anti-diabetic agent; a non-berberine insulin resistance lowering agent; a non-berberine anti-hypertensive agent; or a non-berberine anti-obesity agent, or any other secondary or adjunctive therapeutic agent contemplated herein, using separate formulations or a combinatorial formulation as described above (i.e., comprising both a berberine compound, berberine related, proto-berberine or derivative compound, and a non-berberine therapeutic agent). This coordinate administration may be done simultaneously or sequentially in either order, and there may be a time period while only one or both (or all) active therapeutic agents individually and/or collectively exert their biological activities. A distinguishing aspect of all such coordinate treatment methods is that the berberine compound, berberine related, proto-berberine or derivative compound exerts at least some lipid lowering activity, some glucose lowering activity, and/or some hypertension lowering activity which yields a favorable clinical response in conjunction with a complementary agent, or distinct, clinical response provided by the secondary or adjunctive therapeutic agent. Often, the coordinate administration of the berberine compound, berberine related, proto-berberine or derivative compound with the secondary or adjunctive therapeutic agent will yield improved therapeutic or prophylactic results in the subject beyond a therapeutic effect elicited by the berberine compound, berberine related, proto-berberine or derivative compound, or the secondary or adjunctive therapeutic agent administered alone. This qualification contemplates both direct effects, as well as indirect effects.

Within exemplary embodiments, a berberine compound, berberine related, proto-berberine or derivative compound of Formula I will be coordinately administered (simultaneously or sequentially, in combined or separate formulation(s)), with one or more secondary therapeutic agents, e.g., selected from, for example, anti-hyperlipidemic agents; anti-dyslipidemic agents; plasma HDL-raising agents; anti-hypercholesterolemic agents, including, but not limited to, cholesterol-uptake inhibitors; cholesterol biosynthesis inhibitors, e.g., HMG-CoA reductase inhibitors (also referred to as statins, such as lovastatin, simvastatin, pravastatin, fluvastatin, rosuvastatin, pitavastatin, and atorvastatin); HMG-CoA synthase inhibitors; squalene epoxidase inhibitors or squalene synthetase inhibitors (also known as squalene synthase inhibitors); acyl-coenzyme A cholesterol acyltransferase (ACAT) inhibitors, including, but not limited to, melinamide; probucol; nicotinic acid and the salts thereof; niacinamide; cholesterol absorption inhibitors, including, but not limited to, β-sitosterol or ezetimibe; bile acid sequestrant anion exchange resins, including, but not limited to cholestyramine, colestipol, colesevelam or dialkylaminoalkyl derivatives of a cross-linked dextran; LDL receptor inducers; fibrates, including, but not limited to, clofibrate, bezafibrate, fenofibrate and gemfibrozil; vitamin B6 (also known as pyridoxine) and the pharmaceutically acceptable salts thereof, such as the HCl salt; vitamin B12 (also known as cyanocobalamin); vitamin B3 (also known as nicotinic acid and niacinamide, supra); anti-oxidant vitamins, including, but not limited to, vitamin C and E and betacarotene; angiotensin II receptor (AT₁) antagonist;, renin inhibitors; platelet aggregation inhibitors, including, but not limited to, fibrinogen receptor antagonists, i.e., glycoprotein IIb/IIIa fibrinogen receptor antagonists; hormones, including but not limited to, estrogen; insulin; ion exchange resins; omega-3 oils; benfluorex; ethyl icosapentate; and amlodipine; appetite-suppressing agents or anti-obesity agents including, but not limited to, insulin sensitizers, protein tyrosine phosphatase- 1B (PTP-1B) inhibitors, dipeptidyl peptidase IV (DP-IV) inhibitors, insulin or insulin mimetics, sequestrants, nicotinyl alcohol, nicotinic acid, PPARα agonists, PPAR γ agonists including glitazones, PPARα/γ dual agonists, inhibitors of cholesterol absorption, acyl CoA:cholesterol acyltransferase inhibitors, anti-oxidants, anti-obesity compounds, neuropeptide Y5 inhibitors, β₃ adrenergic receptor agonists, ileal bile acid transporter inhibitors, anti-inflammatories and cyclo-oxygenase 2 selective inhibitors; insulin; sulfonylureas, including but not limited to chlorpropamide, glipizide, glyburide, and glimepiride; cannabinoid antagonists including, but not limited to, rimonabant; camptothecin and camptothecin derivatives, DPP-4 blockers; biguanides, including but not limited to metformin and phenformin; thiazolidinediones including but not limited to rosiglitazone, troglitazone and pioglitazone; alpha-glucosidase inhibitors, including, but not limited to, acarbose and meglitol; D-phenylalanine derivatives; meglitinides; diuretics including, but not limited to, methyclothiazide, hydroflumethiazide, metolazone, chlorothiazide, methyclothiazide, hydrochlorothiazide, quinethazone, chlorthalidone, trichlormethiazide, bendroflumethiazide, polythiazide, hydroflumethiazide, spironolactone, triamterene, amiloride, bumetanide, torsemide, ethacrynic acid, furosemide; beta-blockers including, but not limited to acebutolol, atenolol, betaxolol, bisoprolol, carteolol, metoprolol, nadolol, pindolol, propranolol, and timolol.; angiotensin-converting enzyme (ACE) inhibitors including, but not limited to, benazepril, captopril; enalapril, fosinopril, lisinopril, moexipril, perindopril, quinapril, ramipril, and trandolapril; calcium channel blockers including, but not limited to, amlodipine, diltiazem, felodipine, isradipine, nicardipine sr, nifedipine er, nisoldipine, and verapamil; vasodilators including, but not limited to, nitric oxide, hydralazine, and prostacyclin; angiotensin II receptor blockers including, but not limited to, andesartan, eprosartan, irbesartan, losartan, olmesartan, telmisartan, and valsartan; alpha blockers including, but not limited to, doxazosin, prazosin and terazosin; alpha 2 agonists including, but not limited to clonidine and guanfacine. Such agents may be referred to in whole or in part as metabolic disorder therapeutics, metabolic syndrome therapeutics, anti-obesity therapeutics, anti-hypercholesterolemia therapeutics, anti-diabetic therapeutics, insulin resistance therapeutic agents, anti-hyperglycemia agents, insulin sensitivity increasing agents, anti-hypertensive agents, and/or blood glucose lowering therapeutic agents. Adjunctive therapies may also be used including, but not limited, physical treatments such as changes in diet, psychological counseling, behavior modification, exercise and surgery including, but not limited to, gastric partitioning procedures, jejunoileal bypass, stomach stapling, gastric bands, vertical banded gastroplasty, laparoscopic gastric banding, roux-en-Y gastric bypass, biliopancreatic bypass procedures and vagotomy. Some herbal remedies may also be employed effectively in combinatorial formulations and coordinate therapies for treating metabolic disorders, for example curcumin, gugulipid, garlic, vitamin E, soy, soluble fiber, fish oil, green tea, carnitine, chromium, coenzyme Q10, anti-oxidant vitamins, grape seed extract, pantothine, red yeast rice, and royal jelly.

As noted above, in all of the various embodiments of the invention contemplated herein, the anti-hyperlipidemia and related methods and formulations may employ a berberine compound, berberine related, proto-berberine or derivative compound of Formula I in any of a variety of forms, including any one or combination of the subject compound's pharmaceutically acceptable salts, glycosylated derivatives, isomers, enantiomers, polymorphs, solvates, hydrates, and/or prodrugs. In exemplary embodiments of the invention, berberine is employed within the therapeutic formulations and methods for illustrative purposes.

The pharmaceutical compositions of the present invention may be administered by any means that achieve their intended therapeutic or prophylactic purpose. Suitable routes of administration for the compositions of the invention include, but are not limited to, oral, buccal, nasal, aerosol, topical, transdermal, mucosal, injectable, slow release, controlled release, iontophoresis, sonophoresis, and including all other conventional delivery routes, devices and methods. Injectable methods include, but are not limited to, intravenous, intramuscular, intraperitoneal, intraspinal, intrathecal, intracerebroventricular, intraarterial, subcutaneous and intranasal routes.

The compositions of the present invention may further include a pharmaceutically acceptable carrier appropriate for the particular mode of administration being employed. Dosage forms of the compositions of the present invention include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

If desired, the compositions of the invention can be administered in a controlled release form by use of a slow release carrier, such as a hydrophilic, slow release polymer. Exemplary controlled release agents in this context include, but are not limited to, hydroxypropyl methyl cellulose, having a viscosity in the range of about 100 cps to about 100,000 cps or other biocompatible matrices such as cholesterol.

Compositions of the invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose, dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, which may be prepared by any conventional method of preparing pharmaceutical oral unit dosage forms can be utilized in preparing oral unit dosage forms. Oral unit dosage forms, such as tablets, may contain one or more conventional additional formulation ingredients, including, but not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum, and glyceryl monostearate. Substances which may be used for coating include hydroxypropyl cellulose, titanium oxide, talc, sweeteners and colorants.

Additional compositions of the invention can be prepared and administered in any of a variety of inhalation or nasal delivery forms known in the art. Devices capable of depositing aerosolized purified berberine formulations in the sinus cavity or pulmonary alveoli of a patient include metered dose inhalers, nebulizers, dry powder generators, sprayers, and the like. Methods and compositions suitable for pulmonary delivery of drugs for systemic effect are well known in the art. Additional possible methods of delivery include deep lung delivery by inhalation. Suitable formulations, wherein the carrier is a liquid, for administration, as for example, a nasal spray or as nasal drops, may include aqueous or oily solutions of berberine compositions and any additional active or inactive ingredient(s).

Further compositions and methods of the invention are provided for topical administration of a berberine compound, berberine related, proto-berberine or derivative compound for the treatment of hyperlipidemia. Topical compositions may comprise a berberine compound, berberine related, proto-berberine or derivative compound of Formula I along with one or more additional active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a berberine compound, berberine related, proto-berberine or derivative compound of Formula I dissolved or dispersed in a portion of water or other solvent or liquid to be incorporated in the topical composition or delivery device. It can be readily appreciated that the transdermal route of administration may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example, 24 hours. Transdermal delivery may also be enhanced through techniques such as sonophoresis.

Yet additional berberine compositions of the invention are designed for parenteral administration, e.g. to be administered intravenously, intramuscularly, subcutaneously or intraperitoneally, including aqueous and non-aqueous sterile injectable solutions which, like many other contemplated compositions of the invention, may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers. Additional compositions and formulations of the invention may include polymers for extended release following parenteral administration. The parenteral preparations may be solutions, dispersions or emulsions suitable for such administration. The subject agents may also be formulated into polymers for extended release following parenteral administration. Pharmaceutically acceptable formulations and ingredients will typically be sterile or readily sterilizable, biologically inert, and easily administered. Such polymeric materials are well known to those of ordinary skill in the pharmaceutical compounding arts. Parenteral preparations typically contain buffering agents and preservatives, and injectable fluids that are pharmaceutically and physiologically acceptable such as water, physiological saline, balanced salt solutions, aqueous dextrose, glycerol or the like. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

In more detailed embodiments, compositions of the invention may comprise a berberine compound, berberine related, proto-berberine or derivative compound of Formula I encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively; in colloidal drug delivery systems (for example, liposomes, albumin microspheres, microemulsions, nano-particles and nanocapsules); or within macroemulsions.

As noted above, in certain embodiments the methods and compositions of the invention may employ pharmaceutically acceptable salts, e.g., acid addition or base salts of the above-described berberine compounds and/or berberine related and/or proto-berberine or derivative compounds. Examples of pharmaceutically acceptable addition salts include inorganic and organic acid addition salts. Suitable acid addition salts are formed from acids which form non-toxic salts, for example, hydrochloride, hydrobromide, hydroiodide, sulphate, hydrogen sulphate, nitrate, phosphate, and hydrogen phosphate salts. Additional pharmaceutically acceptable salts include, but are not limited to, metal salts such as sodium salts, potassium salts, cesium salts and the like; alkaline earth metals such as calcium salts, magnesium salts and the like; organic amine salts such as triethylamine salts, pyridine salts, picoline salts, ethanolamine salts, triethanolamine salts, dicyclohexylamine salts, N,N′-dibenzylethylenediamine salts and the like; organic acid salts such as acetate, citrate, lactate, succinate, tartrate, maleate, fumarate, mandelate, acetate, dichloroacetate, trifluoroacetate, oxalate, and formate salts; sulfonates such as methanesulfonate, benzenesulfonate, and p-toluenesulfonate salts; and amino acid salts such as arginate, asparginate, glutamate, tartrate, and gluconate salts. Suitable base salts are formed from bases that form non-toxic salts, for example aluminum, calcium, lithium, magnesium, potassium, sodium, zinc and diethanolamine salts.

To illustrate the range of useful salt forms of berberine compounds, berberine related, proto-berberine and derivative compounds within the methods and compositions of the invention, an exemplary assemblage of salt forms of berberine were produced and tested for their solubility (Table 5). The novel berberine salts thus provided embody yet additional aspects of the invention and exemplify the broad assemblage of useful berberine and related compounds herein.

TABLE 5
Exemplary Berberine Salts

| | | | Amount of | |
|---|---|---|---|---|
| Sample Code | Solvent | Amount of Solute (mg) | Solvent | Solubility Ranking |
| 1. Citrate | Distill | 10 | 7.5 | Slightly Soluble |
| | Water | | | |
| 2. Cysteine | Distill | 10 | 8.8 | Slightly Soluble |
| | Water | | | |
| 3. Acetate | Distill | 10 | 9.0 | Slightly Soluble |
| | Water | | | |
| 4. Lactate | Distill | 10 | 6.0 | Slightly Soluble |
| | Water | | | |
| 5. Nitrate | Distill | 10 | 8.0 | Slightly Soluble |
| | Water | | | |
| 6. Methanesulfonate | Distill | 10 | 1.5 | Slightly Soluble |
| | Water | | | |
| 7. Hydrosulfate* | Distill | 10 | 1.5 | Slightly Soluble |
| | Water | | | |
| 8. Sulfate* | Distill | 10 | 0.5 | Slightly Soluble |
| | Water | | | |
| 9. Salicylate | Distill | 10 | 6.5 | Slightly Soluble |
| | Water | | | |
| 10. Oxalate | Distill | 10 | 6.0 | Slightly Soluble |
| | Water | | | |
| 11. Phosphate | Distill | 10 | 8.0 | Slightly Soluble |
| | Water | | | |
| 12. Formate | Distill | 10 | 8.5 | Slightly Soluble |
| | Water | | | |
| 13. Benzoate | Distill | 10 | 7.0 | Slightly Soluble |
| | Water | | | |
| 14. Tartrate | Distill | 10 | 7.0 | Slightly Soluble |
| | Water | | | |
| 15. Toluenesulfonate | Distill | 10 | 11.0 | Extremely Slightly |
| | Water | | | Soluble |
| 16. Trifluoroacetate | Distill | 10 | 7.5 | Slightly Soluble |
| | Water | | | |
| 17. Control: | Distill | 10 | 10.0 | Extremely Slightly |
| Hydrochloric | Water | | | Soluble |

In other detailed embodiments, the methods and compositions of the invention for employ prodrugs of berberine compounds or berberine related or proto-berberine or derivative compounds of Formula I. Prodrugs are considered to be any covalently bonded carriers which release the active parent drug in vivo. Examples of prodrugs useful within the invention include esters or amides with hydroxyalkyl or aminoalkyl as a substituent, and these may be prepared by reacting such compounds as described above with anhydrides such as succinic anhydride.

The invention disclosed herein will also be understood to encompass methods and compositions comprising a berberine compound, berberine related, proto-berberine or derivative compound of Formula I using in vivo metabolic products of the said compounds (either generated in vivo after administration of the subject precursor compound, or directly administered in the form of the metabolic product itself). Such products may result for example from the oxidation, reduction, hydrolysis, amidation, esterification, glycosylation and the like of the administered compound, primarily due to enzymatic processes. Accordingly, the invention includes methods and compositions of the invention employing compounds produced by a process comprising contacting a berberine compound or berberine related or proto-berberine or derivative compound of Formula I with a mammalian subject for a period of time sufficient to yield a metabolic product thereof. Such products typically are identified by preparing a radiolabelled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The invention disclosed herein will also be understood to encompass diagnostic compositions for diagnosing the risk level, presence, severity, or treatment indicia of, or otherwise managing a hyperlipidemia and/or cardiovascular disease or condition in a mammalian subject, comprising contacting a labeled (e.g., isotopically labeled, fluorescent labeled or otherwise labeled to permit detection of the labeled compound using conventional methods) berberine compound or berberine related or proto-berberine or derivative compound of Formula I to a mammalian subject (e.g., to a cell, tissue, organ, or individual) at risk or presenting with one or more symptom(s) of hyperlipidemia and/or cardiovascular disease, and thereafter detecting the presence, location, metabolism, and/or binding state (e.g., detecting binding to an unlabeled binding partner involved in LDL receptor physiology/metabolism) of the labeled compound using any of a broad array of known assays and labeling/detection methods.

The invention disclosed herein will also be understood to encompass diagnostic compositions for diagnosing the risk level, presence, severity, or treatment indicia of, or otherwise managing a metabolic disorder disease or condition in a mammalian subject, comprising contacting a labeled (e.g., isotopically labeled, fluorescent labeled or otherwise labeled to permit detection of the labeled compound using conventional methods) berberine compound or berberine related or proto-berberine or derivative compound of Formula I to a mammalian subject (e.g., to a cell, tissue, organ, or individual) at risk or presenting with one or more symptom(s) of metabolic disorders, and thereafter detecting the presence, location, metabolism, and/or binding state (e.g., detecting binding to an unlabeled binding partner involved in InsR receptor physiology/metabolism) of the labeled compound using any of a broad array of known assays and labeling/detection methods.

The invention disclosed herein will further be understood to encompass diagnostic compositions for diagnosing the risk level, presence, severity, or treatment indicia of, or otherwise managing a hyperglycemic disease or condition in a mammalian subject, comprising contacting a labeled (e.g., isotopically labeled, fluorescent labeled or otherwise labeled to permit detection of the labeled compound using conventional methods) berberine compound or berberine related or proto-berberine or derivative compound of Formula I to a mammalian subject (e.g., to a cell, tissue, organ, or individual) at risk or presenting with one or more symptom(s) of hyperglycemia, and thereafter detecting the presence, location, metabolism, and/or binding state (e.g., detecting binding to an unlabeled binding partner involved in InsR receptor physiology/metabolism) of the labeled compound using any of a broad array of known assays and labeling/detection methods.

The invention disclosed herein will additionally be understood to encompass diagnostic compositions for diagnosing the risk level, presence, severity, or treatment indicia of, or otherwise managing insulin resistance in a mammalian subject, comprising contacting a labeled (e.g., isotopically labeled, fluorescent labeled or otherwise labeled to permit detection of the labeled compound using conventional methods) berberine compound or berberine related or proto-berberine or derivative compound of Formula I to a mammalian subject (e.g., to a cell, tissue, organ, or individual) at risk or presenting with one or more symptom(s) of insulin resistance, and thereafter detecting the presence, location, metabolism, and/or binding state (e.g., detecting binding to an unlabeled binding partner involved in InsR receptor physiology/metabolism) of the labeled compound using any of a broad array of known assays and labeling/detection methods.

The invention disclosed herein will also be understood to encompass diagnostic compositions for diagnosing the risk level, presence, severity, or treatment indicia of, or otherwise managing a hypertensive disease or condition in a mammalian subject, comprising contacting a labeled (e.g., isotopically labeled, fluorescent labeled or otherwise labeled to permit detection of the labeled compound using conventional methods) berberine compound or berberine related or proto-berberine or derivative compound of Formula I to a mammalian subject (e.g., to a cell, tissue, organ, or individual) at risk or presenting with one or more symptom(s) of hypertension, and thereafter detecting the presence, location, metabolism, and/or binding state (e.g., detecting binding to an unlabeled binding partner involved in InsR receptor physiology/metabolism) of the labeled compound using any of a broad array of known assays and labeling/detection methods.

The invention disclosed herein will further be understood to encompass diagnostic compositions for diagnosing the risk level, presence, severity, or treatment indicia of, or otherwise managing diabetes in a mammalian subject, comprising contacting a labeled (e.g., isotopically labeled, fluorescent labeled or otherwise labeled to permit detection of the labeled compound using conventional methods) berberine compound or berberine related or proto-berberine or derivative compound of Formula I to a mammalian subject (e.g., to a cell, tissue, organ, or individual) at risk or presenting with one or more symptom(s) of diabetes, and thereafter detecting the presence, location, metabolism, and/or binding state (e.g., detecting binding to an unlabeled binding partner involved in InsR receptor physiology/metabolism) of the labeled compound using any of a broad array of known assays and labeling/detection methods.

The invention disclosed herein will also be understood to encompass diagnostic compositions for diagnosing the risk level, presence, severity, or treatment indicia of, or otherwise managing a metabolic syndrome disease or condition in a mammalian subject, comprising contacting a labeled (e.g., isotopically labeled, fluorescent labeled or otherwise labeled to permit detection of the labeled compound using conventional methods) berberine compound or berberine related or proto-berberine or derivative compound of Formula I to a mammalian subject (e.g., to a cell, tissue, organ, or individual) at risk or presenting with one or more symptom(s) of metabolic syndrome, and thereafter detecting the presence, location, metabolism, and/or binding state (e.g., detecting binding to an unlabeled binding partner involved in InsR receptor physiology/metabolism) of the labeled compound using any of a broad array of known assays and labeling/detection methods.

In exemplary embodiments, a berberine compound or berberine related or proto-berberine or derivative compound of Formula I is isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴c, ¹⁵N, ¹⁸O, ¹⁷O, ³¹P, ³²P, ³⁵S, ¹⁸F, and ³⁶Cl, respectively. The isotopically-labeled compound is then administered to an individual or other subject and subsequently detected as described above, yielding useful diagnostic and/or therapeutic management data, according to conventional techniques.

### EXAMPLES

The experiments described below demonstrate novel and powerful uses for a berberine compounds and berberine related and derivative compounds as cholesterol lowering drugs that can effectively lowers serum cholesterol, triglycerides and LDL through a mechanism other than that used by current hypolipidemic drugs, such as statins. In exemplary experiments, cells from a human hepatoma-derived cell line, HepG2, were treated for 24 hours with 700 compounds isolated from Chinese herbs. RNA was then isolated from the cells and analysis of LDLR mRNA was determined using semi-quantitative RT-PCR assays. Of the compounds tested, berberine demonstrated the greatest increase in LDLR expression. Treating HepG2 cells cultured in medium containing 0.5% lipoprotein-depleted fetal bovine serum or serum supplemented with sterols and berberine caused time dependent increases in the expression of LDLR mRNA.

The experiments further demonstrate the novel and powerful uses for berberine compounds and berberine related and derivative compounds in decreasing insulin resistance, increasing glucose consumption, and decreasing serum insulin. These experiments further demonstrate that berberine acts on the insulin receptor (InsR) through a second pathway that differs from the pathway that leads to an increase in LDLR expression. In exemplary experiments, HepG2 cells treated with berberine had an increased expression of InsR. Additionally, both hyperglycemic rats and humans treated with berberine had decreased levels of blood glucose and increased levels of InsR. These and additional findings are further expanded and elucidated within the following examples.

### Example I

### Effects of Berberine on the Levels of Cholesterol, Triglycerides and LDL Protein in a Hyperlipidemia Chinese Hamster

Two weeks prior to treatment, female Chinese hamsters purchased from the National Institute of Vaccine and Serum research (Beijing, China) were switched to a high fat and cholesterol diet (10% lard, 10% egg yolk powder and 1% cholesterol). After two weeks, groups of 14 hamsters were given either 10 mg/kg/day of berberine through peritoneal injection, 20 mg/kg/day of berberine through peritoneal injection, 50 mg/kg/day, 100 mg/kg/day of berberine orally or saline for ten days. Serum cholesterol, triglyceride and LDL levels were measured after 4 h fasting before, during and after the course of the treatment. Four hours after the course of the treatment, the animals were sacrificed and their livers removed for analysis.

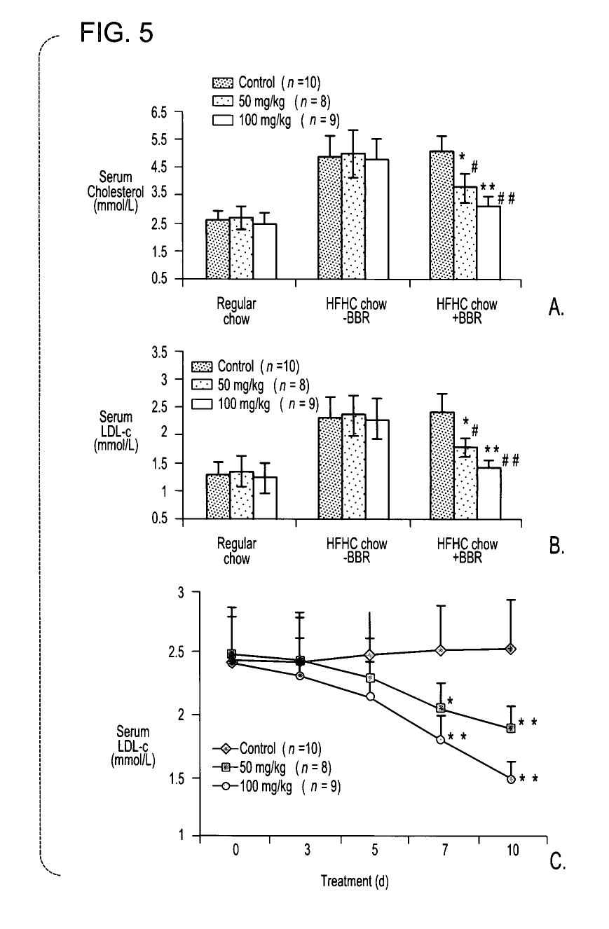

As can be seen in Table 6, berberine decreased the levels of cholesterol, triglycerides and LDL protein in all of the treated animals. After the 10 day treatment, a dose of 50/mg/kg/day of berberine reduced LDL by 26% and a dose of 100 mg/kg/day reduced LDL by 42%. Reductions in serum LDL were observed by day 5 and became significant by day 7 at both doses (FIG. 5).

TABLE 6
Lipid lowering effects of berberine in hyperlipidemia Chinese hamster

| Treatment | | n | Cholesterol | Triglycerides | LDL Protein |
|---|---|---|---|---|---|
| Saline | (control | 14 | 6.4 ± 1.0 | 3.6 ± 0.4 | 2.8 ± 0.9 |
| | group) | | | | |
| Berberine | 10 mg/kg/day | 14 | 4.1 ± 0.7** | 2.6 ± 0.3 | 1.6 ± 0.3** |
| | (peritoneal | | | | |
| | injection) | | | | |
| | 20 mg/kg/day | 14 | 3.2 ± 0.5*** | 1.7 ± 0.5* | 0.9 ± 0.1*** |
| | (peritoneal | | | | |
| | injection) | | | | |
| | 50 mg/kg/day | 8 | 3.5 ± 0.5 | | 2.07 ± 0.9 |
| | 100 mg/kg/day | 14 | 3.8 ± 0.7** | 1.9 ± 0.4* | 1.2 ± 0.2** |
| | (oral) | | | | |

*P < 0.05; **P < 0.01; ***P < 0.001 (compared to the control group)

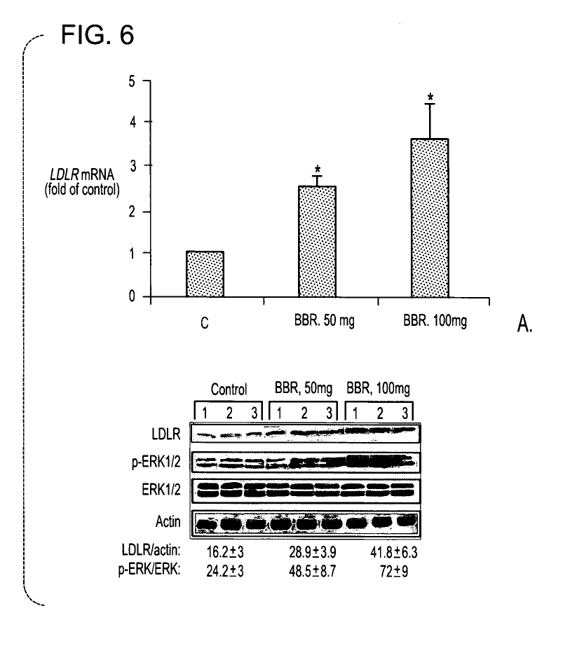

At the end of treatment, three animals from each group were killed and liver LDLR mRNA and protein expressions were examined by quantitative real-time RT-PCR and western blot analysis. For the real-time RT-PCR, reverse transcription with random primers using Superscript II at 42° C. for 30 minutes with 1 μg of total RNA was performed using the ABI Prism 7900-HT Sequence Detection System and Universal MasterMix (Applied Biosystems, Foster City, Calif.). LDLR and GAPD mRNA expression levels were determined using the human LDLR and GAPD Pre-developed TaqMan Assay Reagents (Applied Biosystems). As can be seen in FIG. 6, LDLR mRNA and protein levels were elevated in all berberine treated hamsters in a dose dependent manner. There was a 3.5 fold increase in mRNA and a 2.6 fold increase in protein in hamster livers treated with 100 mg/kg/day of berberine.

### Example II

### Effects of Berberine in Humans with Hyperlipidemia

Human patients with hyperlipidemia (52 males and 39 females) were randomly divided into two groups and treated with either 0.5 g of berberine hydrochloride twice a day (n=63) or a placebo (n=28) for three months. After three months, fasting serum concentrations of cholesterol, triglycerides, HDL and LDL were measured using standard blood lipid tests. Liver and kidney functions were also measured. Those treated with berberine had statistically significant lower cholesterol, triglycerides and LDL protein levels than those treated with the placebo, with berberine hydrochloride lowering serum levels of cholesterol by 18% (P<0.001), triglycerides by 28% (P<0.001) and LDL by 20% (P<0.001). Because some participants were taking other medications that could have influenced the results, the results were reanalyzed using only the data from those participants who were neither on drugs nor special diets before or during berberine therapy. As can be seen in Table 7, the results of those who were only taking berberine hydrochloride were even more significant with serum levels of cholesterol decreasing by 29% (P<0.0001), triglycerides by 35% (P<0.0001) and LDL by 25% (P<0.0001). Berberine was well tolerated by all subjects and no side effects were observed with the exception of one patient having mild constipation during treatment, which was relieved after reducing the dose to 0.25 g twice per day. BBR did not change kidney functions (as determined by measurements of creatine, blood urea nitrogen, and total bilirubin in treated and placebo subjects), but substantially improved liver function—reducing levels of alanine aminotransaminase, aspartate aminotransaminase, and gamma glutamyl transpeptidase, by approximately 48%, 36%, and 41%, respectively. The placebo group showed no significant changes in these parameters.

TABLE 7
Lipid lowering effects of berberine in hyperlipidemia patients

| | Berberine treatment | Berberine Group^{a} | Placebo Group |
|---|---|---|---|
| | (3 months) | (n = 32) | (n = 11) |
| Serum level of | | >5/2 mmol/L | >5.2 mmol/L |
| cholesterol | | | |
| Cholesterol | Before | 5.9 ± 0.7 | 6.0 ± 0.8 |
| (mmol/L) | | | |
| | After | 4.2 ± 0.9* | 5.8 ± 0.6 |
| Triglycerides | Before | 2.3 ± 1.8 | 2.2 ± 0.7 |
| (mmol/L) | | | |
| | After | 1.5 ± 0.9* | 2.0 ± 1.0 |
| LDL Protein | Before | 3.2 ± 0.7 | 3.7 ± 0.7 |
| (mmol/L) | | | |
| | After | 2.4 ± 0.6*** | 3.7 ± 0.8 |
| HDL Protein | Before | 1.1 ± 0.3 | 1.2 ± 0.5 |
| | After | 1.1 ± 0.3 | 1.2 ± 0.4 |

^{a}Statistical analysis of the baselines of cholesterol, trigylceride, HDL-c, and LDL-c showed that there were no significant differences between the berberine and placebo groups before therapy (p > 0.05). ***P < 0.0001 as compared to baselines of before treatment group (matched t test)

### Example III

### The Effect of Berberine on LDLR Expression

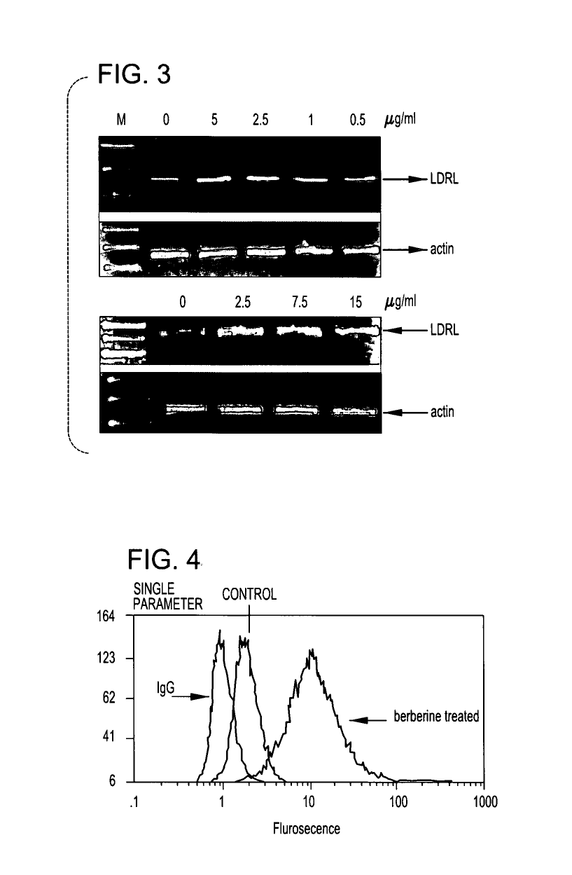

Bel-7402 cells were treated with 0, 0.5, 1, 2.5, 5, μg/ml of berberine or 2.5, 7.5 and 15 μg/ml of berberine sulfate. The cells were then centrifuged and washed and LDLR mRNA was extracted. LDLR mRNA levels were then measured using scan quantitative RT-PCR, (FIGS. 3 A and B). As can be seen in FIGS. 3 A and B, treatment with berberine and berberine sulfate increased LDLR mRNA expression in a dose dependent fashion with 5 μg/ml berberine increasing LDLR mRNA expression 2.3 fold. Berberine also increased LDLR protein expression on the surface of BEL-7402 cells.

Bel-7402 cells treated with 5 μg/ml of berberine were detached with cell removal buffer containing EDTA, washed and resuspended in FACS solution (PBS with 0.5% BSA and 0.02% sodium azide) at a density of 1×10⁶ cells/ml. Cells were then incubated with monoclonal antibody to LDLR (Santa Cruz Biotechnology, Inc., Santa Cruz, Calif.) at a final dilution of 1:50 and left at room temperature for 1 hour. The cells were then reacted with isotope matched, nonspecific mouse IgG as a control for nonspecific staining. The cells were then washed and stained with FITC conjugated goat antibody to mouse IgG (Santa Cruz Biotechnology, Inc., Santa Cruz, Calif., 1:100 dilution) and the fluorescence intensity was analyzed by FACS (FACSort, Becton Dickinson, Franklin Lakes, N.J.). As can be seen in FIG. 4, berberine increased cell surface LDLR protein expression 4 times.

The above studies demonstrate that berberine's serum lipid lowering effect is mediated through an increase in LDLR expression.

### Example IV

### Use of Berberine and Simvastatin in Combination to Lower Serum Lipid Levels in Rats

Rats were fed a high fat high cholesterol (HFHC) diet for 10 days, and then divided into groups of seven. The rats were then administered berberine or simvastatin, or a combination of berberine and simvastatin orally for 25 days. After 25 days, serum cholesterol, triglyceride and LDL-c levels were measured. As can be seen in Table 8, treatment with berberine significantly decreased the cholesterol, triglycerides and LDL-c levels in the rats and was more effective than simvastatin in lowering triglyceride and LDL-c levels. The combination of simvastatin, and berberine lowered the cholesterol, triglyceride and LDL-c levels further than either alone.

TABLE 8
Combination treatment with berberine and simvastatin in rats

| | | | Total | | |
|---|---|---|---|---|---|
| | | Daily dose | Cholesterol | Triglyceride | LDL-c |
| Experiment Group | N | (oral, mg/kg) | (mmol/L) | (mmol/L) | (mmol/L) |
| Normal Control Group | 7 | no | 2.6 ± 0.4 | 1.9 ± 0.6 | 1.4 ± 0.3 |
| Untreated hyper- | 7 | no | 6.4 ± 0.6 | 3.2 ± 0.3 | 2.4 ± 0.3 |
| lipidemia Group | | | | | |
| Berberine Treatment | 7 | 100 | 3.8 ± 0.5 | 2.1 ± 0.3 | 1.2 ± 0.2 |
| Group | | | | | |
| Simvastatin Treatment | 7 | 25 | 3.6 ± 0.4 | 2.5 ± 0.3 | 1.4 ± 0.1 |
| Group | | | | | |
| Berberine + Simvastatin | 7 | 100 + 25 | 2.5 ± 0.4 | 1.6 ± 0.2 | 1.1 ± 0.2 |
| Group | | | | | |

After 25 days, blood total cholesterol, triglyceride and LDL-c levels were examined. The results in tables are average ± standard error.

### Example V

### Use of Berberine to Increase LDLR mRNA Stability

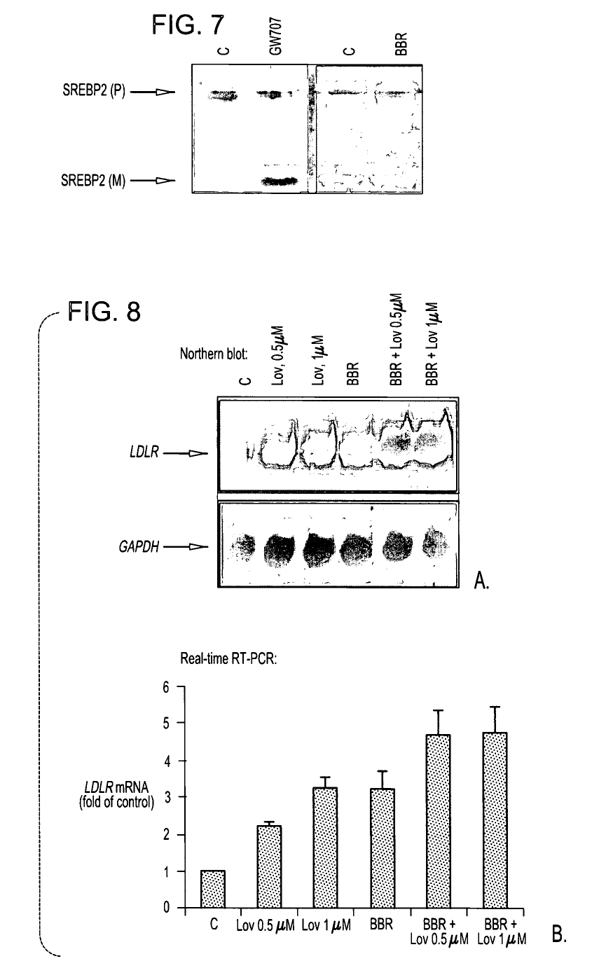

HepG2 cells were cultured with either berberine hydrochloride or GW707 as a positive control for 8 hours. Total cell lysates from untreated cells or cells treated with either berberine or GW707 were then harvested and analyzed by Western blot. As can be seen in FIG. 7, GW70 substantially increased the amount of the mature form of SREB-2, whereas berberine had no effect. These data indicate that berberine effectively increases LDLR expression by a mechanism distinct from that used by statins, thereby further evincing that this novel drug and its related and derivative compounds will provide useful anti-hyperlipidemic formulations and methods with minimal side effects attributed to other known anti-hyperlipidemic drugs.

### Example VI

### Function of Berberine in the Presence of Statins

HepG2 cells were cultured in LPDS medium and were then untreated, treated with lovastatin at 0.5 and 1 μM concentrations with or without berberine for 24 hours, or were treated with berberine alone. As can be seen in FIG. 8, berberine and lovastatin had additive stimulation effects on LDLR mRNA expression, which data evince general utility of the novel, combinatorial formulations and coordinate treatment methods describe herein above.

### Example VII

### Analysis of LDLR Promoter Activity

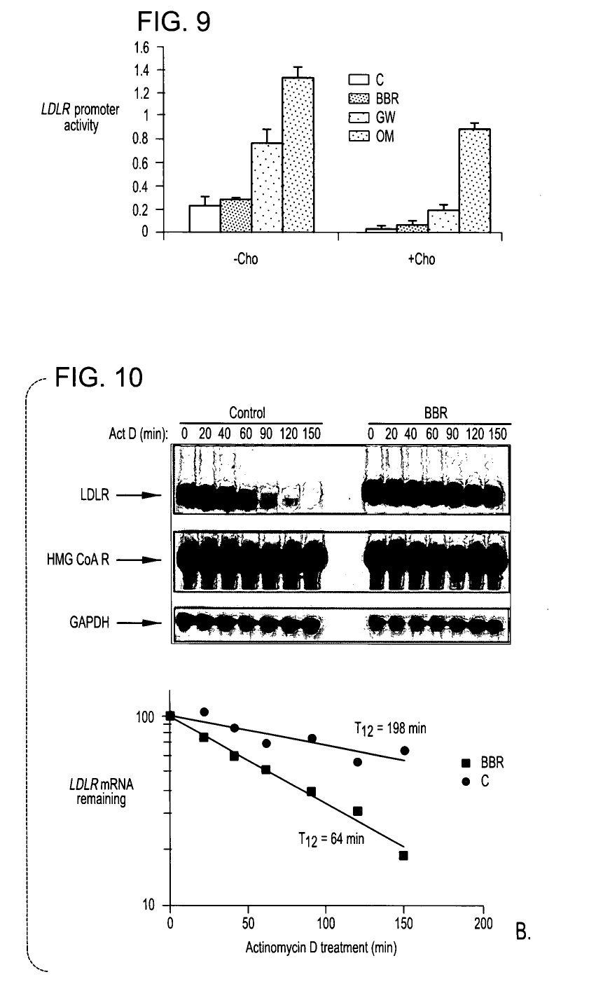

HepG2 cells were transfected with the reporter construct pLDLR234Luc, which contains the SRE- I motif and the sterol-independent regulatory element that mediates the cytokine oncostatin M-induced transcription of the LDLR gene. After transfection, cells were culture in 0.5% lipoprotein depleted fetal bovine serum (LPDS) or LPDS and cholesterol medium followed by an 8 hour treatment with berberine, GW707 or oncostatin M. As can be seen in FIG. 9, LDLR promoter activity was strongly elevated by GW707 and oncostatin M under both culturing conditions. Berberine had no effect, further evincing that this compound operates via a different mechanism of LDLR regulation compared to other known drugs possessing anti-hyperlipidemic activity.

### Example VIII

### Stabilization of LDLR mRNA by Berberine

HepG2 cells were cultured and then left alone or treated with berberine for 15 hours. After 15 hours, actinomycin D (5 μg/ml) was added to cells at 0, 20, 40, 60, 90, 120, or 150 minutes. Total mRNA was isolated and analyzed by Northern blot for the amount of LDLR mRNA. As can be seen in FIG. 10, berberine prolonged the turnover rate of LDLR transcript by approximately threefold. In contrast, the mRNA stability of HMG-CoA reductase was not altered by berberine.

### Example IX

### Transfection of HepG2 Cells

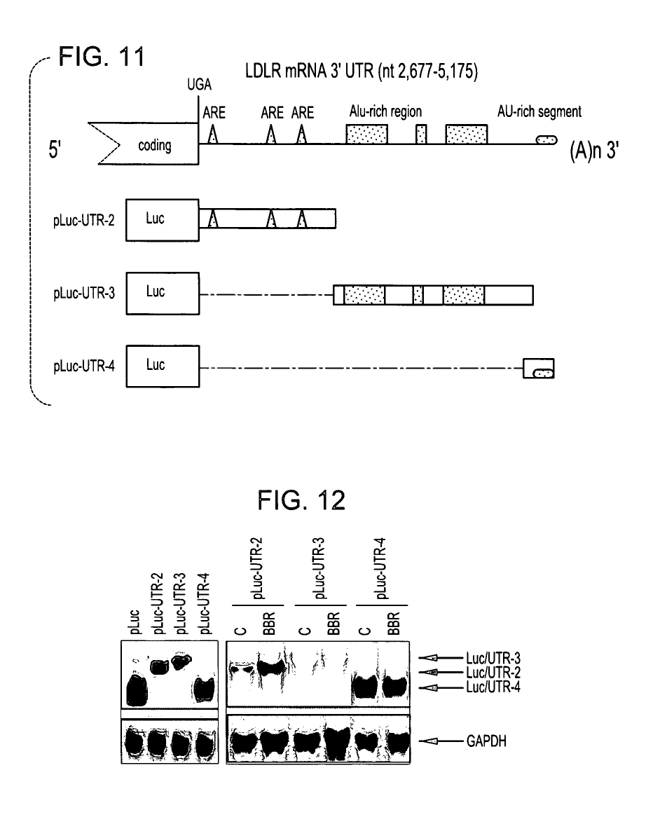

Three consecutive fragments of LDLR 3 ′UTR were inserted into a cytomegalovirus promoter driven Luc plasmid (pLuc) at the 3′ end of the Luc coding sequence before the SV40 polyadenylation signal. The wild-type Luc reporter plasmid pLuc was constructed by insertion of the Luc cDNA into the HindIII and Xba sites of pcDNA3.1/Zeo(+). Addition of the LDLR 3/UTR was accomplished by PCR amplifying different regions of the 2.5 kb 3′UTR of LDLR mRNA using XbaI-tailed primers and pLDLR3 as the template. The wild type pLuc and the chimeric plasmids pLuc-UTR-2, UTR-3 and UTR-4 were transfected into HepG2 cells (FIG. 11). Cells seeded in culture dishes were transiently transfected with the chimeric plasmids. Twenty-four hours after transfection, cells were trypsinized and reseeded equally into two dishes for each plasmid transfection. After overnight incubation, one dish was treated with dimethylsulfoxide as the solvent control and another was treated with berberine for eight hours. To detect the presence of Luc-LDLR fusion transcripts, a PCR reaction was performed to amplify a 550 base pair fragment of Luc coding region with 5′ primer Luc-2up (5′-GCTGGAGAGCAACTGCARAAGGC-3′) (SEQ ID NO:1) and the 3′ primer Luc-21o (5′-GCAGACCAGTAGATCCAGAGG-3′) (SEQ ID NO:2) using pGL3-basic as the template. The PCR fragment was labeled with ³²P and used in the northern blot analysis to measure expression of Luc mRNA and Luc-LDLR 3′UTR chimeric fusion. As can be seen in FIG. 12, inclusion of UTR-2 and UTR-3 sequences reduced expression levels of Luc mRNA by approximately 3-4 fold, indicating the presence of destabilization determinants within these groups whereas the Luc mRNA levels were only moderately reduced by fusing with UTR-4. Berberine increased the level of Luc-UTR-2 mRNA by 2.5 fold without affecting expressions of LucUTR-3 and Luc-UTR-4 or the wild type. This demonstrates that berberine affected the mRNA stability of the heterologous Luc-LDLR transcript and that the stabilization is mediated through regulatory sequences present in the 5′ proximal region of the LDLR 3′UTR (nt 2677-3582).

### Example X

### Determination of the Role of ARE and UCAU Motif in Berberine Mediated LDLR mRNA Stabilization

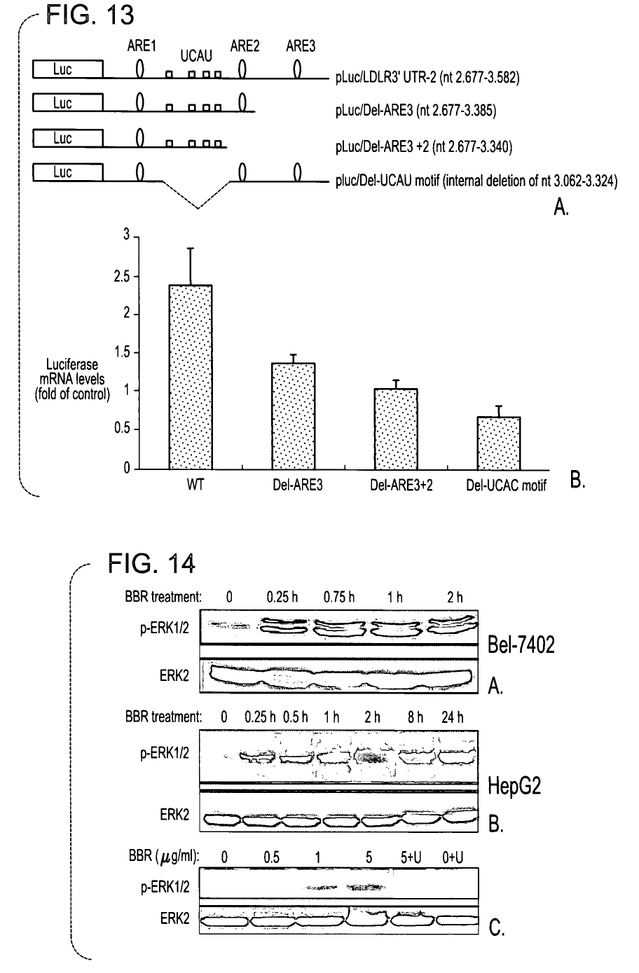

To create ARE deletion constructs, an Apa site at nt 3,384 was generated for deleting ARE3, and an Apa1 site at nt 3,334 for deleting ARE2 by site-directed mutagenesis using pLuc/UTR-2 as the template. Mutated plasmids were cut with Apa1 to remove the ARE-containing region and then the remaining vector was religated with the 5′ proximal region of UTR-2. To create the UCAU motif deletion, two SacII sites for internal deletion of nt 3.062-3,324 were generated using UTR-2 as the template. All constructs were sequenced and the correct clones were further propagated to isolate plasmid DNA. These constructs and the berberine responsive wild-type construct were transfected into HepG2 cells. The effects of berberine on the chimeric Luc transcripts were determined by measuring Luc mRNA using a quantitative real-time RT-PCR assay. Deletion of the ARE3 region resulted in a partial loss of berberine stimulation and deletion of both the ARE3 and ARE2 rendered the construct unresponsive to berberine. The stabilizing effect of berberine on the Luc transcript was also abolished by deleting the UCAU motifs. (FIG. 13).

### Example XI

### Activation of the MEK1-ERK Pathway by Berberine

HepG2 or Bel-7402 cells were treated with berberine for 0.25, 0.5, 0.75, 1, 2, 8, and 24 hours respectively and tested for levels of activated ERK by western blotting using antibodies that only recognize the activated (phosphorylated) ERK. In both hepatoma cell lines, berberine rapidly activated ERK and the kinetics of ERK activation preceded the upregulation of LDLR expression by berberine (FIGS. 14A and B). The activation of berberine is also dose dependent (FIG. 14C). These data indicate that activation of ERK pathway is a prerequisite event in the berberine mediated stabilization of the LDLR transcript.

### Example XII

### Pharmacokinetics of Berberine

Healthy human volunteers were given 300 mg of berberine orally. Blood samples were taken 0.5, 1, 2, 3, 4, 5, 7, 12 and 24 hours after administration and evaluated for berberine concentration by HPLC. The blood concentration curve was analyzed by 3P87 Pharmacokinetics Software program (Chinese Pharmacological Association, China). Using a one compartment model, the median pharmacokinetic parameter estimates (ranges) were as follows: Peak Time: Tpeaking: 2.37 hr, peak concentration: Cmax: 394.7 ηg/ml, vanishing half life: T1/2: 2.91 h, the area under the curve AUC: 2799.0 μg/L h, clear rate CL: 130.5 L/h. The average drug retention time was 32.63 hours.

In parallel animal model studies, four canine (beagle) subjects were given 45 mg/kg of berberine orally. Serum concentrations of the drug were determined by HPLC at 2 and 3 hours after administration. There was no obvious spectrum peak detected suggesting that the concentration was below the minimum detection limit of 10 μg/ml. One dog receiving 280 mg/kg of berberine had a berberine peak showing a concentration of 31.4 μg/ml after two hours and 22.6 ηg three hours after administration. After the berberine had cleared the system, the same dog was then administered 700 mg/kg of berberine and blood samples were taken 2, 3, 5, 7, 9 and 24 hours after administration resulting in concentrations of 21.51, 44.89, 49.54, 36.35, 27.83, and 16.01 ηg/ml respectively.

Four beagles were injected intravenously with 100 mg/kg of berberine. Using a two compartment model, the pharmacokinetic parameter estimates were as follows: Vanishing half life T1/2B is 12.59±8.83 h. Area under the curve AUC is 1979.31±1140.31 μg/h.L; blood clearance rate: CL is 60.70±24.38 L/h.

In additional, parallel animal model studies, 3H-berberine was administered intravenously to 5 rabbits (25 MBq/kg) and through intravenous drip to four rabbits (46.25 Mbq/kg). 0.1 ml of blood was removed at various times and radiation emissions were measured. The pharmacokinetic parameter estimates for both groups were as follows: T1/2α respectively: 1.41±0.16 h, 1.03±0.11 h, and T1/2β respectively: 35.3±1.3 h, 35.8±2.0 h, Vd respectively 20±3 L/kg and 22.1±1.7 L/kg.

50 mg/kg of berberine was administered through stomach infusion to six rabbits. Serum samples were taken at various points after administration and RP-HPLC was used to measure the drug concentration. The blood drug concentration-time data was analyzed using the 3P87 pharmacokinetics software program (Chinese Pharmacological Association, China). Using automated fitting, the rabbit berberine pharmacokinetics model was found to match with the One Compartment Open model. The main pharmacokinetics parameters were as follows: peak time Tpeak: 0.63±0.25 h, peak concentration Cmax: 92.72±50.89 ηg/ml, vanishing half life: T1/2β: 3.11±0.58 h, the area under the curve, AUC: 491.7±295.5 μg h/L. The results indicate that berberine can be absorbed rapidly to reach the effective concentration.

The rabbit blood protein binding rate was measured by in vitro dialysis at a rate of 38±3% (XD±S, n=6).

In yet additional animal models studies, mice were injected in the tail vein with 3H-berberine (135 LBq/10 g). Tissue radiation emission was measured 5 minutes to 2 hours after administration with the distribution of the berberine concentrations from highest to lowest being: lung>liver>kidney>spleen>heart>intestine>stomach>brain.

In a final series of animal model studies, rats were orally administered 3H-berberine. Forty-eight hours after administration, excretions were tested for the presence of berberine. 2.7% of the oral dose was measured in the urine and 86% of the oral dose was measured in the fecal matter.

Rats received intravenous berberine (9.25 MBq/kg). Six days accumulation of rat urine and fecal secretions were measured for the presence of berberine. 73% of the intravenous dose of the berberine was found in the accumulated urine in both metabolized and unmetabolized forms. 10.9% of the intravenous dose was found in the fecal matter.

Three rats were given berberine (9.25 MBq/kg) intravenously. After 24 hours, gall bladder secretions were collected and evaluated for the presence of berberine. There was 10.1±0.9% (x±SD, n=3) of berberine in the gall bladder secretions.

### Example XIII

### Toxicity Analysis of Berberine

Rats and mice were administered berberine through a variety of techniques, including orally, through subcutaneous injection, peritoneal injection and intravenous injection.

In rats, toxicity was achieved with an oral dose of LD₅₀>15000 mg/kg. Toxicity through subcutaneous injection was LD₅₀ 7970-10690 mg/kg. Toxicity through peritoneal injection was LD₅₀=138.1-146.2 mg/kg and LD₅₀ 46.2-63.3 mg/kg when the berberine was administered through intravenous injection.

In mice, toxicity was achieved with an oral dose of LD₅₀>29586-4500 mg/kg. Toxicity through subcutaneous injection was LD₅₀ 13.9-20 mg/kg. Toxicity through peritoneal injection was LD₅₀ 30-32.2 mg/kg and LD₅₀ 7.6-10.2 mg/kg with intravenous injection.

For long term toxicity determination, rats were administered 300 mg/kg of berberine orally for 182 days. No abnormalities were found in blood tests, blood biochemistry, urine analysis or histopathology

To assess teratologic potential, pregnant mice were orally administered a daily dose of between 30-480 mg/kg of berberine beginning on day 7 of the pregnancy and continuing for seven days. Rats were administered berberine beginning on day 9 of their pregnancy for seven days. No birth defects were evident.

### Example XIV

### Exemplary Combinatorial Therapy Employing Berberine and Lovastatin

In accordance with the above teachings, combinatorial drug therapy employing a berberine compound or berberine related or derivative compound of Formula I, in combination with an exemplary, secondary anti-hyperlipidemia agent, was demonstrated using berberine and an exemplary statin, lovastatin, in rat model subjects. The procedures for this study accord with those of the foregoing example, and the results are provided in Table 9, below.

TABLE 9
Combinatoral anti-hyperlipidemia efficacy of berberine and lovastatin in a coordinate
treatment regimen
Cholesterol and LDL concentration in mmol/L

| | Cholesterol | Cholesterol | LDL | LDL | Number | P |
|---|---|---|---|---|---|---|
| Treatment | Day 0 | Day 15 | Day 0 | Day 15 | of rats | value |
| Normal diet | 1.35 | 1.30 | 0.8 | 0.85 | 5 | |
| High fat diet | 3.6 | 3.5 | 2.2 | 2.1 | 9 | |
| High fat diet and | 3.65 | 2.75 | 2.25 | 1.7 | 9 | <0.05 |
| Berberine | | | | | | |
| (80 mg/kg/day) | | | | | | |
| High fat diet and | 3.6 | 2.7 | 2.15 | 1.62 | 9 | <0.05 |
| Lovastatin | | | | | | |
| (10 mg/kg/day) | | | | | | |
| High fat diet and | 3.8 | 2.6 | 2.3 | 1.55 | 11 | <0.01 |

Berberine + lovastatin
Day 0 represents: untreated rats
Day 15 represents: rats treated for 15 days

The foregoing data evince combinatorial effectiveness of an exemplary berberine compound employed in a coordinate treatment protocol with a secondary anti-hyperlipidemia agent, in accordance with the teachings herein above.

### Example XV

### Effects of Berberine on Blood Glucose in Rats

Nineteen Wistar rats (Male, 250g, the Institute of Experiment Animal Sciences, Chinese Academy of Medical Sciences, Beijing) were fed high fat and high cholesterol (HFHC) diet containing 25% lard, 20% sucrose and 5% yolk powder (Institute of Experiment Animal Sciences) for 4 weeks to induce insulin resistance status. Then, after fasting for 24 hours, 20 mg/kg of streptozotocin (Sigma) dissolved in 0.01 M citric acid buffer (pH 4.3) was injected via the tail vein. A week later, rats with blood glucose concentrations over 11.1 mmol/L were considered hyperglycemic and divided into two groups of 7 and one group of 5 as the control.

The rats in the two groups of 7 were treated with berberine orally twice a day for 14 days, at 8:00 am and 5:00 pm, with a total dose of berberine at 75 mg/kg/day or 150 mg/kg/day, respectively. Blood samples were taken by tail snip after 4 hours fasting on indicating days of treatment, and blood glucose was measured. On the last day of the experiment, all of the rats fasted overnight and were then sacrificed.

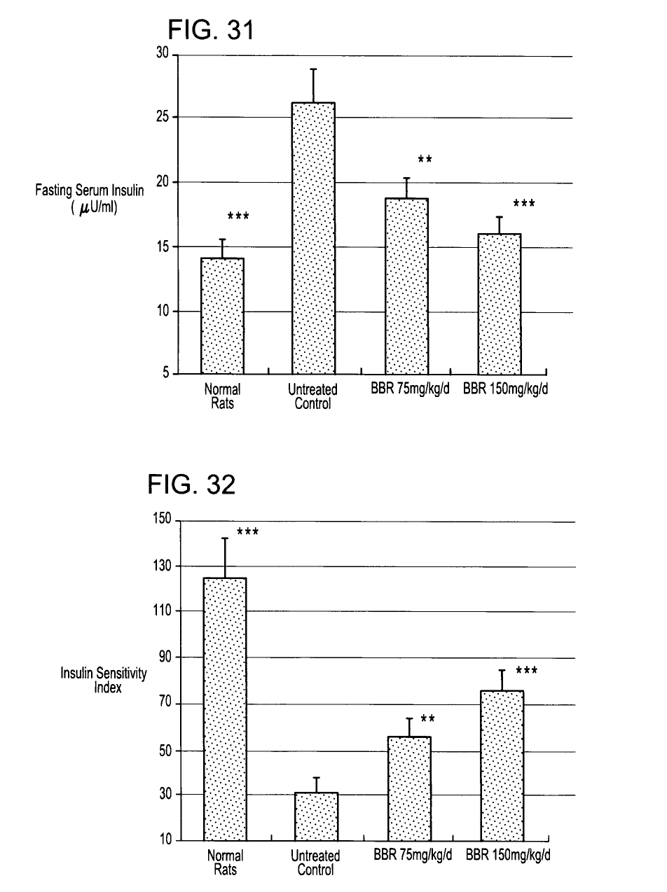

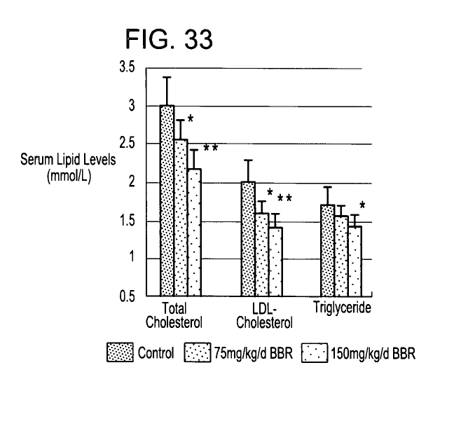

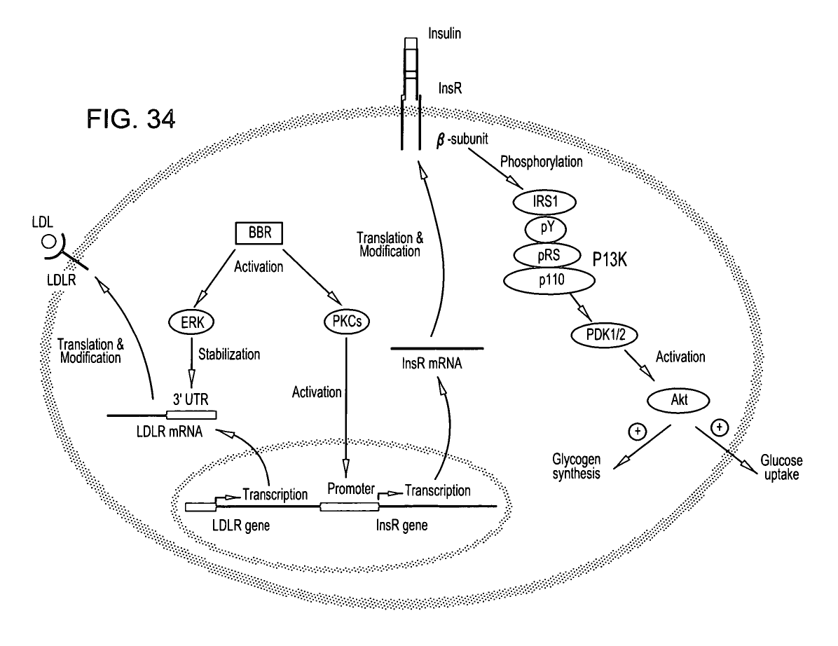

The livers were dissected and stored in liquid nitrogen for RNA extraction, real-time RT-PCR and PKC activity assay. Total blood samples were also collected to assay fasting blood glucose and serum insulin levels. The insulin levels were analyzed using radio-immunoassay (Linco Research, St Charles, Mo.). The insulin sensitivity indexes (ISI) were calculated according to the formula: 10⁴/(fasting serum insulin X fasting blood glucose) (Hanson, Am. J. Epidemiol. 15 1(2), 190-198 (2000)). The insulin level and ISI of normal rats were also determined for comparison. As can be seen in FIG. 32, the HFHC diet significantly reduced insulin sensitivity (p<0.001). It also elevated the fasting blood glucose from 7 to 12.8 mmol/L (P<0.001). Treatment for 14 days with berberine resulted in dose-dependent declines in fasting blood glucose with a dose of 75 mg/kg/day reducing glucose by 22% (p<0.01), and 150 mg/kg/day lowering glucose by 33% as compared to untreated rats on the same HFHC diet (FIG. 27).

The time-dependent effect of berberine was also observed (FIG. 27), with decreases in blood glucose observed by day 5 and statistically significant by day 9 at both 75 mg/kg/day and 150 mg/kg/day (p<0.05, <0.01).

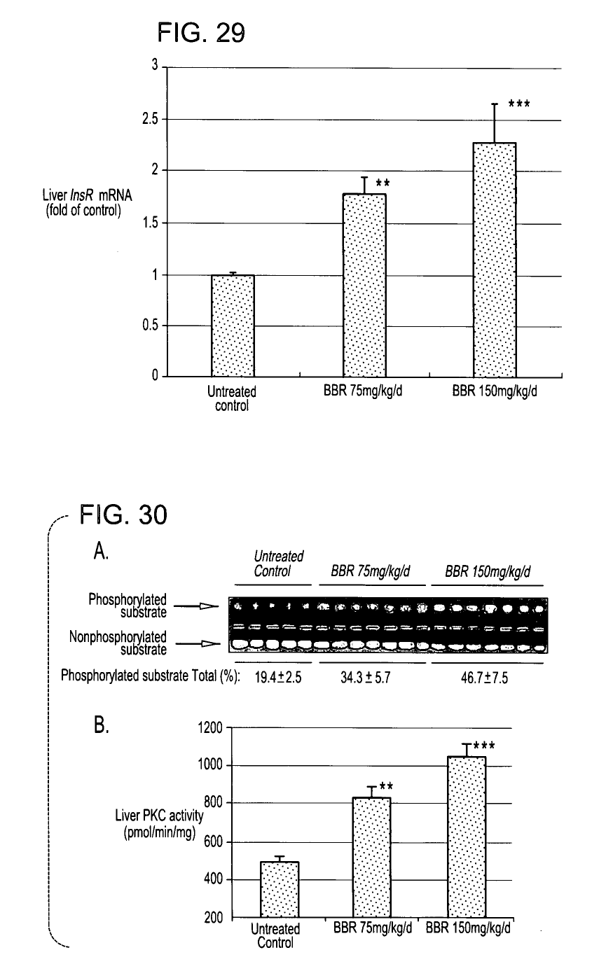

Liver mRNA extracts were used for quantitative real time RT-PCR assay. Total cellular RNA was reverse-transcribed into cDNA using the Reverse Transcription System (Promega, Madison, Wis.). Quantitative real-time PCR were performed with these cDNA using the Applied Biosystems 7500 Real-Time PCR System and TaqMan® Universal PCR Master Mix (Applied Biosystems, Foster City, Calif.). All of the 20× TaqMan® Gene Expression Assay reagents containing gene-specific primers and TaqMan® probes for human or rat InsR, ACTB and LDLR were purchased from Applied Biosystems. 2.5 μl of cDNA sample, 12.5 μl of Universal PCR Master Mix, and 1.25 μl of TaqMan® Gene Expression Assay reagent were mixed in a 25 μl reaction system, and the comparative CT method was used in relative gene quantification using the TaqMan® SDS analysis software. As can be seen in FIGS. 28 and 29, InsR mRNA was elevated in all berberine treated rats in a dose-dependent manner with a 1.8- and 2.3-fold increase in InsR mRNA in the livers of rats treated with 75 and 150 mg/kg/day respectively (p<0.01, <0.001). (Results represent the mean ±sd of liver InsR mRNA and LDLR mRNA of each group.)

These results were concurrent with the increased activity of PKC in the livers of berberine treated rats (FIG. 30). PKC activity was analyzed using the PepTag® Assay for Non-Radioactive Detection of Protein Kinase-C or cAMP-Dependent Protein Kinase kit (Promega) according to the protocol. Briefly, after homogenization and partial purification, 10 of sample protein was mixed with 5 μl of PepTag® Cl peptide (specific substrate of PKC), 5 μl of reaction buffer and 5 μl of PKC activator solution in a 25 μl reaction system. The reactions were performed at 30° C. for 30 minutes. Then, the samples were loaded onto a 0.8% agarose gel. After electrophoresis, the phosphorylated and nonphosphorylated PepTag® Cl peptide were separated, with the phosphorylated ones negatively charged. The gels were photographed under an UV light. The bands containing the phosphorylated substrate were then excised and melted. They were transferred to a 96-well plate and quantified using densitometry according to the supplier's protocol. The catalytic activity of total PKC of a specific sample was expressed as pmol/min/mg, representing the number of picomoles of phosphate transferred to the substrate per minute per milligram of proteins of the sample.

Additionally, the fasting serum insulin in the rats was measured. As shown in FIGS. 31 and 32 (**p<0.01 and ***p<0.001), untreated rats fed a HFHC diet for 4 weeks demonstrated a significant increase of fasting serum insulin and a decrease of the insulin sensitivity index (ISI) in comparison to the normal controls, indicating insulin resistance in the animals. As can be seen in FIG. 31, treatment of the animals with berberine significantly reduced the serum insulin levels in the insulin resistant rats as well as improved the ISI (FIG. 32), suggesting a restoration of the impaired insulin sensitivity and a reduction of insulin resistance.

The lipid profile of the rats was also measured after the 14 day treatment with berberine. As can be seen in FIG. 33, 150 mg/kg/day of berberine reduced cholesterol by 25%, LDL-c by 33% and triglyceride by 24% (p<0.01, **0.01 and *0.05 respectively), as compared to the control animals administered the same HFHC diet. The therapeutic efficacy observed in this animal model reflects a synergistic effect of berberine on InsR and LDLR, which antagonizes insulin resistance and significantly improves sugar- and lipid- metabolism in vivo.

### Example XVI

### Effects of Berberine on Metabolic Syndrome

Twenty-eight patients 28 (male/female, 17/11; age, 57±8 y) were diagnosed as having metabolic syndrome if they met three or more of the following criteria: fasting blood glucose>7 mmol/L, serum triglyceride>1.7 mmol/L, fasting blood insulin>13 mmcU/mL, blood pressure>135/85 and/or BMI>23. (Eckel, Lancet 365 (9468), 1415-28 (2005); Executive Summary, JAMA 285, 2486-2497 (2001); Balkau, Diabet. Med. 16, 442-443 (1999)) The patients were enrolled in this study at the Nanjing First Hospital, Nanjing, China. The patients were requested to end previous medications or therapies for at least two weeks prior to the beginning of the study.

The patients were then given 1 gram of berberine per day, orally for two months. Blood samples were taken both before and after two months of berberine treatment. Fasting blood levels of glucose, LDL-c, cholesterol, HDL-c, triglyceride and blood insulin were measured using standard methods routinely applied in hospitals. Liver and kidney functions were also monitored in the patients.

As can be seen in Table 10, treatment with berberine reduced fasting blood glucose from 10.7±0.56 to 8.0±0.45 mmol/L (Mean ±SE, p<0.001), blood insulin from 17±2.15 to 12.1±1.35 mmol/L (p<0.05), triglyceride from 2.0±0.22 to 1.47±10.14 mmol/L (p<0.001), cholesterol from 6.1±±0.11 to 4.7±0.16 mmol/L (p<0.0001), blood pressure from 156/86 to 133/79 (p<0.01 for both) and body-mass-index (BMI) from 23.8±0.63 to 23.1±0.6 (p<0.01).

TABLE 10
Therapeutic effect of berberine in patients with metabolic syndrome

| | Patients with metabolic syndrome^{# (n = 28)} | | |
|---|---|---|---|
| Clinical measurement* | Before BBR treatment | After BBR treatment | P |
| FBG (<7.0, mmol/L) | 10.7 ± 0.56 | 8.0 ± 0.45 | 0.00027 |
| Blood Insulin (2.5-13.0, mlU/L) | 17 ± 2.15 | 12.1 ± 1.35 | 0.028 |
| Blood Triglyceride (<1.7, mmol/L) | 2.0 ± 0.22 | 1.47 ± 0.14 | 0.001 |
| Blood Cholesterol <5.2, (mmol/L) | 6.1 ± 0.11 | 4.7 ± 0.16 | 0.0001 |
| BMI** | 23.8 ± 0.63 | 23.1 ± 0.6 | 0.007 |
| Blood Pressure | 156/86 | 133/79 | 0.01(for both) |

FBG: Fasting blood glucose.
^{#}BBR treatment, 1 g/day, Bid, 2 months; Values are means ± SEs.
*Nomial range and units.
**Asians at BMI of 23-24 has equivalent risk of metabolic syndrome as a BMI of 25-29.9 in white people.
BMI: Body-Mass-Index.

### Example XVII

### Effect of Berberine on InsR Expression in Human Hepatoma Cell Lines

Cells from the human hepatoma cell line HepG2 were incubated with 0, 1, 2.5, 5, 10 or 15 μg/ml of berberine respectively for eight hours. Total cellular RNA was isolated using the Ultraspec RNA lysis solution (Biotecxs Laboratory, Houston, Tex.) following the vender's protocols. 10 pg of the RNA sample was transferred to nitrocellulose membrane via a slot-blot apparatus (Schleicher & Schuell, Keene, N.H.). The blots were fixed by baking at 80° C. for 2 h, followed by hybridization to a 0.89-kb long, 32P-labelled human InsR cDNA probe. The same membranes were then stripped and re-hybridized to a human ACTB probe as internal control. Quantitative real time RT-PCR assays were also done. For the RT-PCR assay, total cellular RNA was reverse-transcribed into cDNA using the Reverse Transcription System (Promega, Madison, Wis.). Quantitative real-time PCR were performed with these cDNA using the Applied Biosystems 7500 Real-Time PCR System and TaqMan® Universal PCR Master Mix (Applied Biosystems, Foster City, Calif.). All of the 20× TaqMan® Gene Expression Assay reagents containing gene-specific primers and TaqMan® probes for human or rat InsR, ACTB and LDLR were purchased from Applied Biosystems. 2.5 μl of cDNA sample, 12.5 μl of Universal PCR Master Mix, and 1.25 μl of TaqMan® Gene Expression Assay reagent were mixed in a 25 μl reaction system, and the comparative CT method was used in relative gene quantification using the TaqMan® SDS analysis software. InsR mRNA levels were corrected by measuring ACTB mRNA levels.

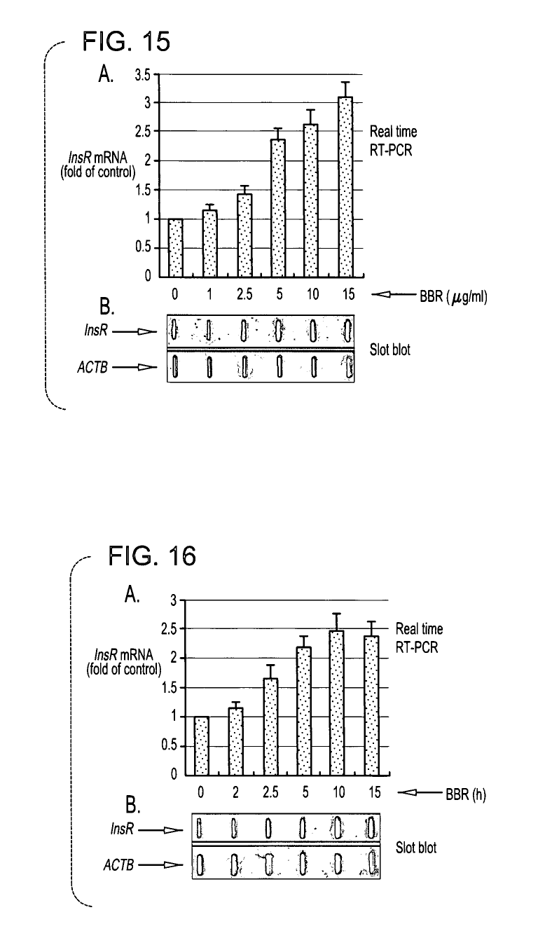

As can be seen in FIG. 15, berberine and showed a dose dependent increase in the expression of InsR mRNA. Using the abundance of InsR mRNA in untreated cells as a baseline of 1 and plotting the amount of InsR mRNA from berberine treated cells relative to that value, quantitative real time RT-PCR showed a 40% increase of InsR mRNA in cells treated with 2.5 μg/ml of berberine for 8 hours and a maximal increase of 3.2-fold of the control was seen with a concentration of 15 μg/ml (FIG. 15A). A similar magnitude of increase in InsR mRNA level was confirmed by the slot blot (FIG. 15B).

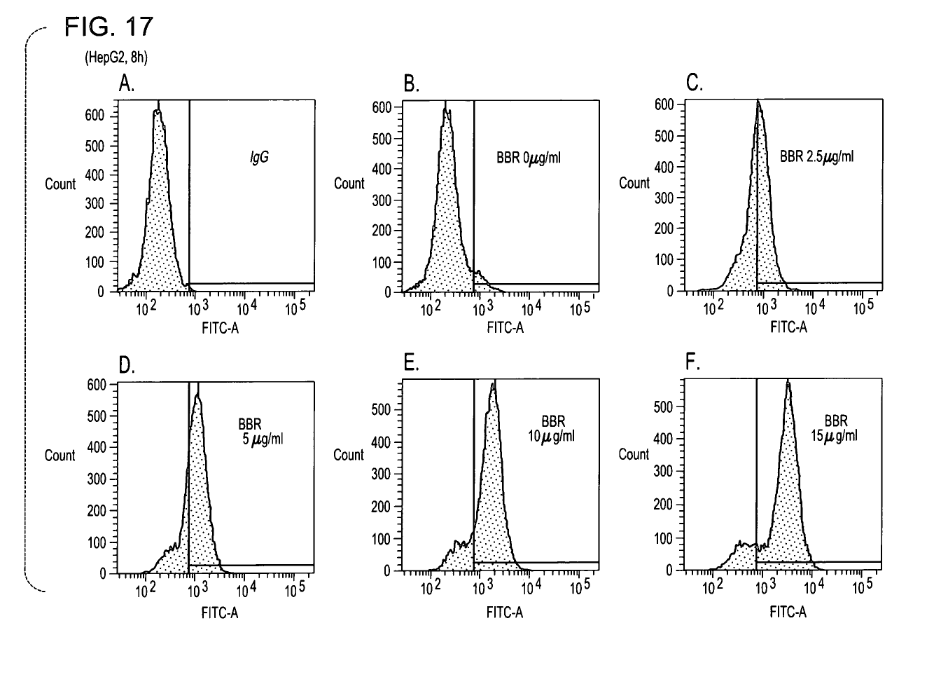

The effect of berberine was also time-dependent. HepG2 cells were treated with 7.5 μg/ml for 0, 2, 4, 6, 8 or 24 hours and total RNA was isolated as described above for slot blot and RT-PCR assays of InsR mRNA and ACTB mRNA expression. The level of InsR mRNA increased 4 h after exposure of cells to berberine and reached the peak level of 2.5-fold of the control at 8 h; the expression of InsR mRNA remained high for at least 24 h (FIG. 16). HepG2 cells cultured with 0, 2.5, 5, 10 or 15 μg/ml of berberine or normal mouse IgG (FIG. 17) for eight hours then detached, washed and resuspended in FACS solution (PBS with 0.5% BSA and 0.02% sodium azide) at a density of 1×10⁶ cells/ml. Cells were then incubated with a monoclonal antibody to InsR (NeoMarkers, Fremont, Calif.) at a final dilution of 1:100 (room temperature, 0.5 h). An isotype-matched, nonspecific mouse IgG was used as a control for nonspecific staining. Then, cells were washed and stained with FITC conjugated goat-anti-mouse IgG (Santa Cruz Biotech, 1:200 dilution). The fluorescent intensity was examined by flow cytometry (FAC Sort, Becton Dickinson, Calif.). As can be seen in FIG. 17, berberine increased cell surface expression in Caucasian lever cell line HepG2.

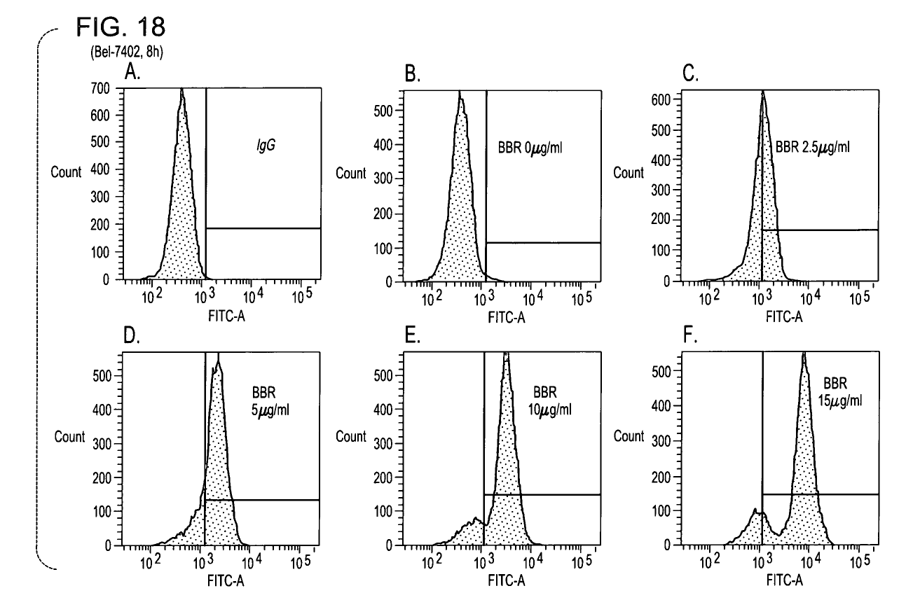

The effect of berberine on InsR was further confirmed in another hepatoma cell line, Bel-7402 of Chinese origin (FIG. 18). The results showed an identical upregulating effect of berberine on InsR expression in cells with either Asian (Bel-7402) or Caucasian (HepG2) genetic background. The expression of PPAR-r mRNA in liver cells was not changed by berberine (data not shown).

The increased InsR expression directly translated into an enhanced InsR sensitivity in target cells. Glucose consumption of human hepatic cells treated with insulin was significantly increased by berberine (**p<0.01, n=4; FIG. 19A). In order to determine the role of InsR in this effect, siRNA was used to silence InsR gene decreasing the level of InsR mRNA expression and decreasing the effectiveness of berberine on increasing InsR mRNA levels. (FIG. 19B)

Human InsR siRNA duplex, non-specific control siRNA, siRNA transfection reagent and siRNA transfection medium were obtained from Santa Cruz Biotechnology, Inc. (Santa Cruz, Calif.) and the vendor's siRNA transfection procedure was followed. Briefly, HepG2 cells were seeded onto six-well plates in antibiotic-free RPMI-1640 medium containing 10% fetal bovine serum (FBS). After 60-80% confluence of monolayer, FBS containing medium was discarded and cells were washed with siRNA transfection medium (2 mVwell). 0.8 ml of siRNA transfection medium containing 6 μl of human InsR siRNA duplex (or control siRNA) and 6 ld¹ of siRNA transfection reagent were well mixed at room temperature for 30 min, followed by loading onto washed HepG2 cells. After an 8 hr incubation, the transfection mixture was removed and fresh RPMI-1640 medium supplied with 10% FBS was added. The cells were then incubated for 24 hr. Then, the culture medium was replaced with fresh medium and incubated for an additional 24 hr. At the end of incubation, FBS containing medium was removed and replaced with serum-free fresh RPMI-1640 medium. 7.5 μg/ml of berberine and/or 0.5 nM of insulin were added to the InsR siRNA transfected or untransfected cells. After 12 hr incubation, the amount of glucose in the sample medium was determined. The glucose consumption was calculated according to the following formula: glucose level of the fresh RPMI-1640 minus glucose level of the used RPMIN-1640. Inhibition of InsR mRNA and protein expression by InsR siRNA was confirmed by either real-time RT-PCR or Western-Blot analysis.
¹ Please check that this is the correct unit. It should probably be μl?

As can be seen in FIG. 19, silencing InsR with siRNA in the liver cells strongly inhibited InsR mRNA expression and completely abolished its effect on insulin-related glucose consumption (FIG. 19 A). The results indicate that the presence of InsR on the cell surface and insulin in the circulation are both essential for berberine to increase the cellular consumption of glucose. Therefore, berberine antagonizes insulin resistance and improves cellular response to insulin through upregulating the expression of InsR on the cell surface. This effect on InsR together with the action on LDLR renders berberine useful in the treatment of sugar- and lipid-metabolic disorders.

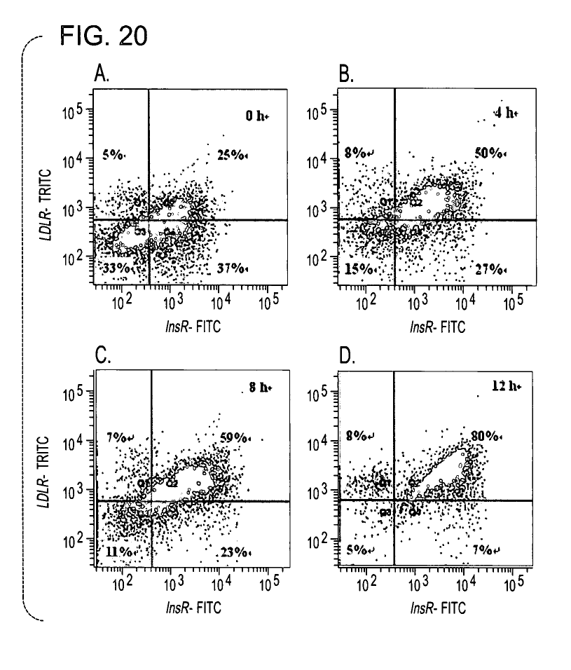

To view the simultaneous increase of the expression of LDLR and InsR by berberine, double-immune staining was conducted. HepG2 cells were treated with 7.5 μg/ml of berberine for 0, 4, 8 or 12 hours, then detached and fixed in 4% paraformaldehyde solution. Cells were then washed and treated with PBA (PBS with 5% BSA) on ice for 15 min. After discarding the supernatant, cells were resuspended in PBA and incubated simultaneously with a monoclonal antibody against InsR (Santa Cruz Biotech. Inc., Santa Cruz, Calif., 1:40) and a rabbit-polyclonal antibody against LDLR (Santa Cruz, 1:20) on ice for 1 hr. Normal mouse IgG and rabbit IgG were used as controls. After incubation, cells were washed with PBA followed by staining with a FITC-conjugated goat-anti-mouse IgG (Santa Cruz, 1:100), as well as a TRITC-conjugated goat-anti-rabbit IgG (Santa Cruz, 1:50) on ice for 40 min. After incubation with secondary antibodies, cells were washed twice with PBA and suspended in 4% paraformaldehyde. The fluorescence intensities of FITC/TRITC on cell surface were analyzed by flow cytometry. As seen in FIG. 20, the results demonstrated a remarkable upregulation of both LDLR and InsR in a similar magnitude on the surface of human hepatocytes treated with berberine, suggesting the dual-target bioactivity of berberine for metabolic syndrome.

### Example XVIII

### Effect of Berberine on InsR mRNA Stability

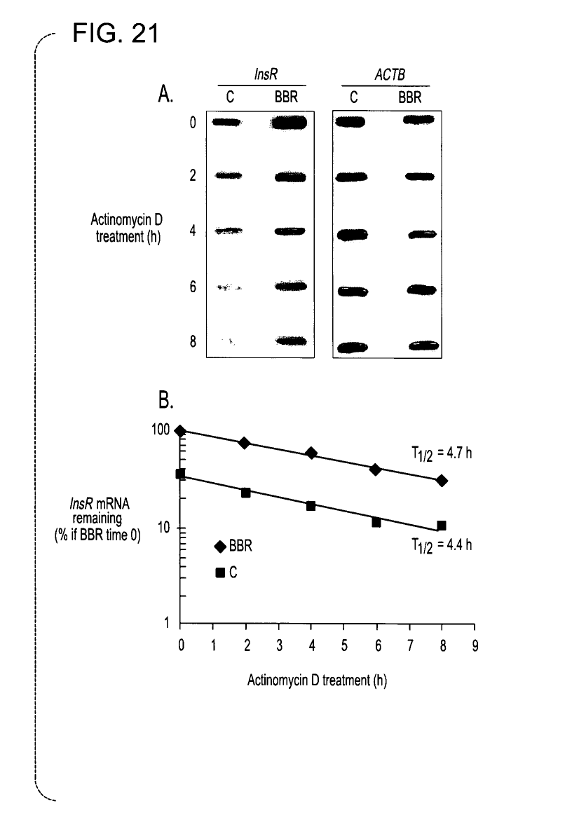

HepG2 cells were treated or untreated with 7.5 μg/ml of berberine for 8 h. Then, 5 μg/ml of actinomycin D was added to block the transcription. Total cellular RNA was harvested at 0, 2, 4, 6, or 8 hours after actinomycin D treatment, and slot-blotted to nitrocellulose membranes as described above. The membranes were respectively hybridized with InsR and ACTB specific probes as described above, and bands were quantitated through densitometry. The InsR mRNA levels were normalized to ACTB, and their remaining percentages are plotted against time and the decaying rate or half-life of InsR mRNA was calculated (FIG. 21B).

As shown in FIG. 21, unlike that of LDLR mRNA, berberine treatment did not prolong the turnover rate of InsR transcript with respect to the untreated cells (4.7 hr vs. 4.4 hr), suggesting that enhanced InsR gene expression by berberine occurs in the transcriptional rather than the post-transcriptional stage.

### Example XIX

### Analysis of InsR Promoter Activity

The InsR gene promoter contains a 1.8 kb long segment (Mitchell, Science 245, 371-378 (1989); Araki J. Biol. Chem. 262, 16186-16191 (1987)). The InsR promoter luciferase gene fusion plasmid (pGL3-1.5kIRP) was kindly provided by Dr. Araki E of the Graduate School of Medical Sciences, Kumamoto University, Honjo, Kumamoto, Japan. In this fusion construct, 1.5 kb fragment of the human insulin receptor gene promoter was inserted into the Hind III site of pGL3-basic vector forming pGL3-1.SkIRP fusion plasmid (Nakamaru, Biochem Biophys Res Commun. 328 (2) 449-454 (2005)).

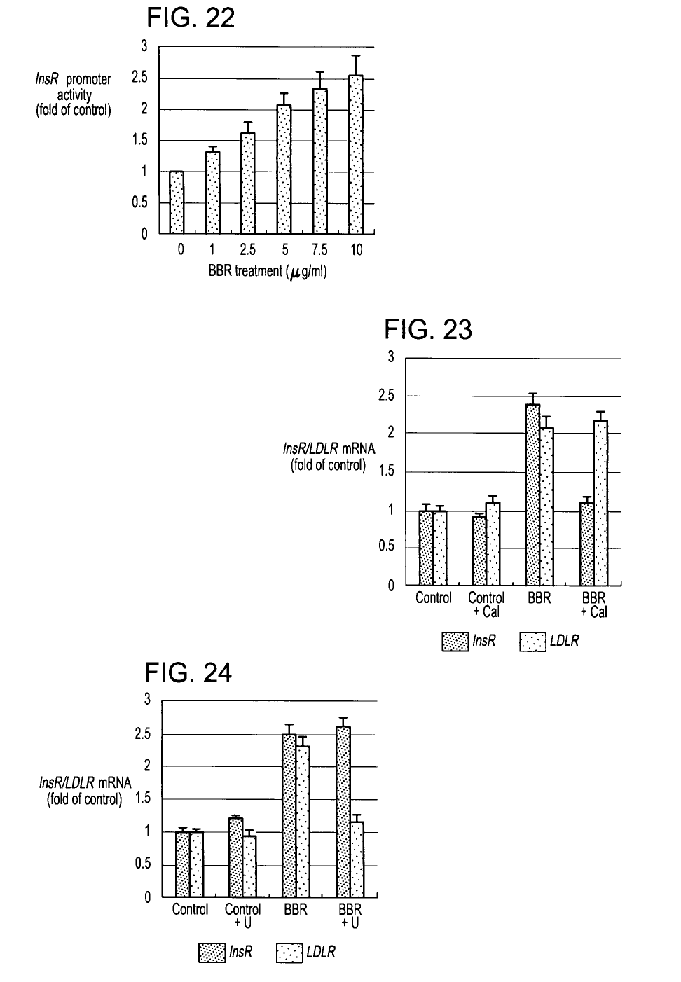

HepG2 cells (2×105) were transfected with 1 μg of the pGL3-1.SkIRP using the FuGENE 6 Transfection Reagent (Roche Applied Science, Indianapolis, Ind.). After overnight incubation, the cells were treated with DMSO as the solvent control or with 0, 1, 2.5, 5, 7.5 and 10 μg/ml berberine for 8 h. Cell lysates were prepared and luciferase activities were measured using the Luciferase Reporter Gene Assay (Roche Applied Science). The experiment was repeated 4 times. As shown in FIG. 22, berberine increased the level of Luc mRNA in the pGL3-1.5kIRP transfected cells at a dose-dependent manner, and at a concentration of 10 μg/ml, berberine elevated Luc mRNA level in the cells by 2.5-fold. The expression of the Luc mRNA in the cells transfected with pGL3 was not affected by berberine (data not shown). The results demonstrate the stimulating effect of berberine on the InsR gene promoter.

### Example XX

### Determination of the Pathway for Berberine-Induced InsR Gene Transcription

To explore the pathway responsible for the berberine-induced InsR gene transcription, different kinase inhibitors were used, including the MEK1-ERK inhibitor U0126, the p38 kinase inhibitor SB203580, the c-Jun N-terminal kinase inhibitor curcumin, the PI-3 kinase inhibitor wortmannin, and the PKC inhibitor calphostin C.

HepG2 cells were pretreated with each of the inhibitors 1 hour prior to treatment with 7.5 μg of berberine for 8 hours. Total RNA was then isolated and the relative amount of InsR and LDLR mRNA was measured by quantitative RT-PCR as described in Example XVII. It was determined that the activity of berberine on InsR gene transcription was most sensitive to the PKC inhibitor calphostin C.

Calphostin C at 0.2 μM completely eliminated the activity of berberine on InsR gene transcription, but did not change the level of LDLR mRNA (FIG. 23). In contrast, inhibition of ERK pathway by U0126 did not effect the activity of berberine on InsR transcription, but completely abolished the increase of LDLR mRNA (FIG. 24). These results indicate that the berberine pathway on InsR gene expression is separate from its effect on LDLR.

### Example XXI

### Activation of the PKC Pathway by Berberine

To determine whether berberine directly activates PKC, pGL3-1.5kIRP transfected HepG2 cells were either not treated, treated with 0.2 μM of calphostin C, 5 μg/ml of berberine or 0.5 μM of PKC activator phorbol-12-myristate-13-acetate (PMA) (Gandino, Oncogene, 5(%), 721-725 (1990) or combinations thereof for eight hours. The activity of total PKC in the control and berberine treated cells was assessed by PKC activity assay.

Cellular PKC activity was analyzed using the PepTag® Assay for Non-Radioactive Detection of Protein Kinase-C or cAMP-Dependent Protein Kinase kit (Promega) according to the protocol. Briefly, after homogenization and partial purification, 10 μl of sample protein was mixed with 5 μl of PepTag® Cl peptide (specific substrate of PKC), 5 μl of reaction buffer and 5 μl of PKC activator solution in a 25 μl reaction system. The reactions were performed at 30° C. for 30 minutes. Then, the samples were loaded onto a 0.8% agarose gel. After electrophoresis, the phosphorylated and nonphosphorylated PepTag® Cl peptide were separated, with the phosphorylated ones negatively charged. The gels were photographed under an UV light. The bands containing the phosphorylated substrate were then excised and melted. They were transferred to a 96-well plate and quantified using densitometry according to the supplier's protocol. The catalytic activity of total PKC of a specific sample was expressed as pmol/min/mg, representing the number of picomoles of phosphate transferred to the substrate per minute per milligram of proteins of the sample.

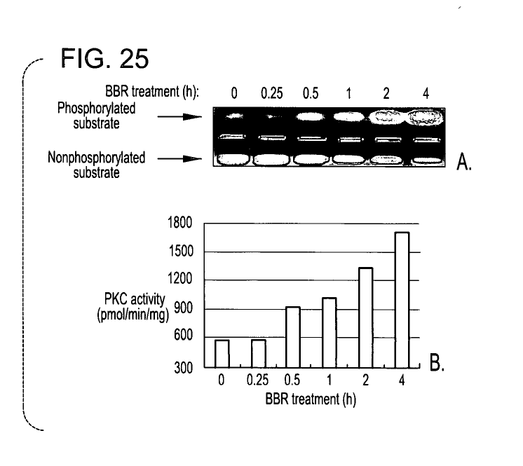

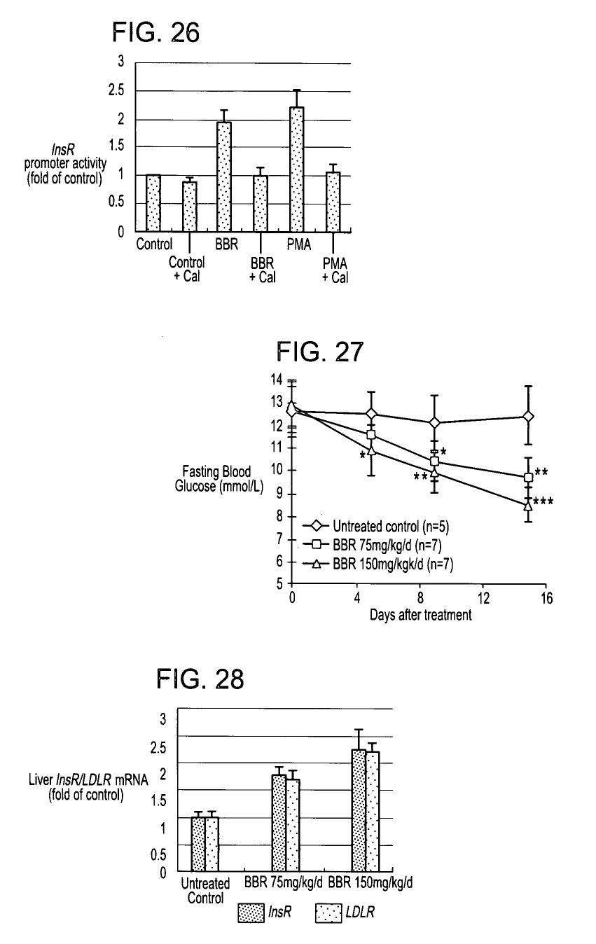

As shown in FIG. 25, PKC activity was increased in liver cells treated with berberine in a time-dependent fashion; the elevation of PKC activity was first observed at 0.5 hr (after berberine treatment) and went up with time. The kinetics of PKC activation preceded the upregulation of InsR expression by berberine. Pre-treatment of the cells with PKC inhibitor Calphostin C diminished the ability of berberine to stimulate the InsR gene promoter (FIG. 26). The PKC activator PMA exhibited the same results confirming that the PKC pathway is a part of the mechanism for the activation of the InsR gene promoter. This result supports the data in FIG. 23, and indicates that activation of PKC pathway is essential for the berberine-mediated upregulation of InsR expression.

### Example XXII

### Effect of Berberine on Blood Glucose in Hyperglycemic Patients with Type 2 Diabetes

Ninety-seven hyperglycemic patients (54 males and 43 females) with fasting blood glucose>7 mmol/L and post-prandial blood glucose>11.1 mmol/L were enrolled in a study at the Nanjing First Hospital in Nanjing, China. The patients were asked to end any current treatments for hyperglycemia at least 2 weeks prior to commencement of the study.

Fifty of the patients (male/female, 27/23; age 57±8y) were randomly assigned to be given 0.5 gram twice a day (1g/day total) of berberine (Nanjing Second Pharmaceutics, Inc., Nanjing, China), orally for 2 months. Out of these 50 patients, 25 had hyperlipidemia, 9 had hypertension and 2 had cardiovascular disease. Twenty-six patients (male/female, 15/11; age 56±11 y) were given 1.5 grams of metformin (Double-Crane Pharmaceutical, Inc., Beijing, China) per day, orally for two months. Of these twenty-six patients, 11 had hyperlipidemia, and four had hypertension. The remaining twenty-one patients (male/female, 11/10, age 49±10 y) were given 4 mg per day of rosiglitazone (Glaxowelcome, UK), orally for two months. Of these 21 patients, ten had hyperlipidemia and four had hypertension. Metformin and rosiglitazone served as reference controls as they are standard treatments for type 2 diabetes. Statistical analysis of the baselines of fasting blood glucose, HbA1c and triglycerides showed no significant differences among the groups prior to treatment (p>0.05).

Blood samples were taken prior to the commencement of therapy and after completion of the therapy. As can be seen in Table 11, berberine significantly lowered the fasting blood levels of glucose by 26% (p<0.0001), hemoglobin A1c (HbA1c) by 18% (p<0.0001) and triglycerides by 18% (p<0.002).

TABLE 11
Effects of Berberine on Fasting Blood Glucose, HbA1c,
and Triglycerides in Patients with Type 2 Diabetes.

| Measurement | | BBR | Metformin | Rosiglitazone |
|---|---|---|---|---|
| (normal range) | Treatment | Type 2 Diabetes | Type 2 Diabetes | Type 2 Diabetes |
| Fasting blood glucose (FBG) | (2 months) | (>7.0 mmol/L, n = 50) | (>7.0 mmol/L, n = 50) | (>7.0 mmol/L, n = 50) |
| FBG | Before | 10.4 ± 0.4 | 10.9 ± 0.5 | 9.1 ± 0.8 |
| (<7.0 mmol/L) | | | | |
| HbA1c | Before | 8.0 ± 0.3 | 9.4 ± 0.5 | 8.3 ± 0.4 |
| (4.0-6.0%) | After | 6.8 ± 0.2*** | 7.2 ± 0.3*** | 6.8 ± 0.3*** |
| Triglyceride | Before | 1.7 ± 0.1 | 1.7 ± 0.2 | 1.9 ± 0.3 |
| (<1.7 mmol/l) | After | 1.4 ± 0.1** | 1.6 ± 0.1 | 1.6 ± 0.1 |

Results are presented as Mean ± SE.
**p < 0.01;
***p < 0.001 as compared to the baseline of “before treatment”.

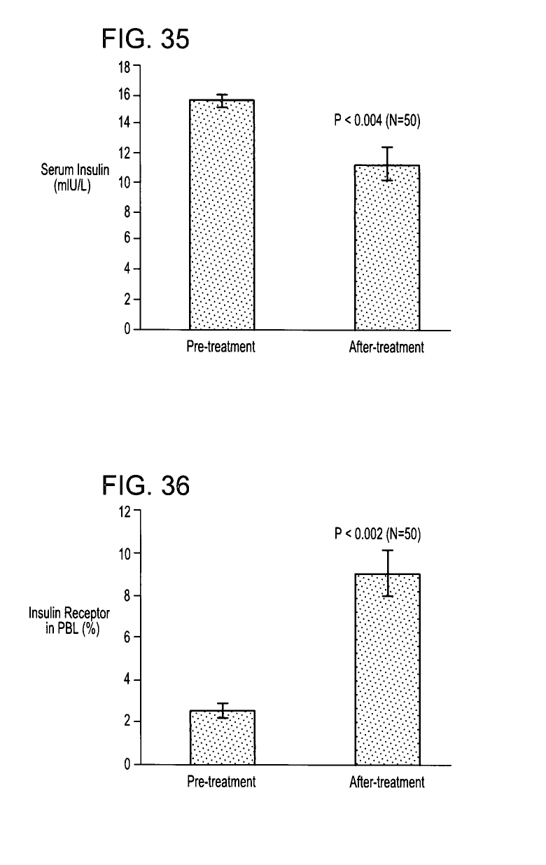

Additionally as seen in FIG. 35, serum insulin levels decreased into the normal range (2.5-13 mlU/L) after treatment with berberine. Although liver enzymes were within normal range before and after berberine treatment, the levels declined with statistical significance in ALT (31±19 vs 23±16, p<0.002) and r-GT (47±26 vs 31±23, p<0.002); kidney function remained stable and in normal range after berberine treatment (BUN, 5.8±1.0 vs 5.7±1.2; Cr, 70±13 vs 71±14).

### Example XXIII

### Determination of an Increase in InsR Expression in Patients Treated with Berberine

Peripheral blood lymphocytes (PBL) were collected for InsR expression analysis. InsR protein expressed on the surface of peripheral blood lymphocytes were stained with monoclonal antibody against human insulin receptor (Pharmagen, San Diego, Calif.) and analyzed in a flow cytometer (BD and Company, San Jose, Calif.)

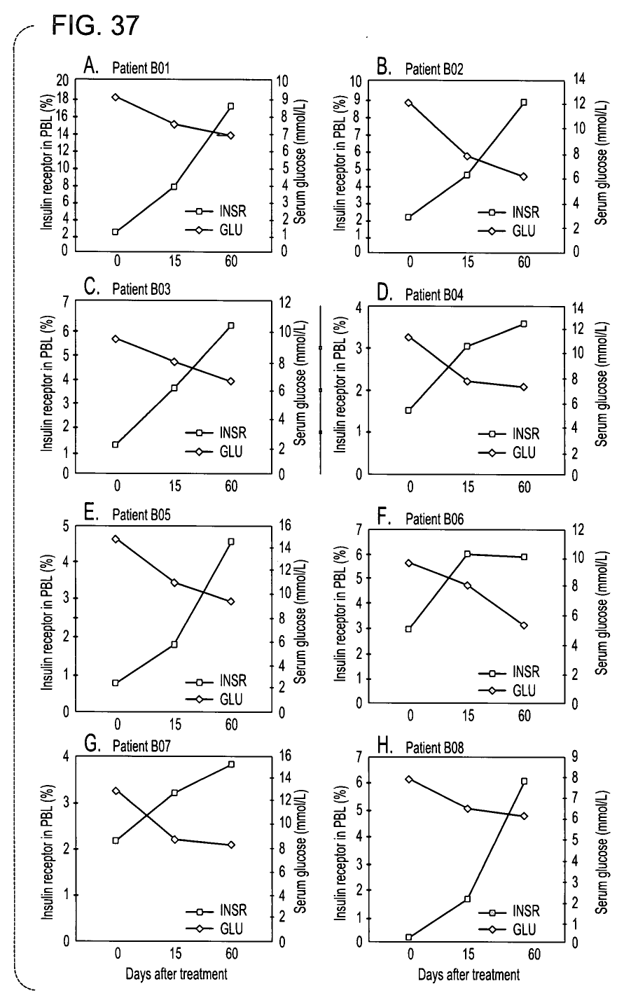

As shown in FIG. 36, the % of PBL expressing InsR on the surface significantly increased after treatment with berberine (p<0.002). Eight patients returned for further testing two weeks after the treatment with berberine ended. As shown in FIG. 37, all of the eight returning patients demonstrated a negative correlation between fasting blood glucose and insulin receptor expression, and the increase of InsR expression on the surface of lymphocytes accompanied a reduction of blood glucose. This is in agreement with the results in the animal experiments in Example XV.

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

### All Disclosure and Description Contained in the Following Publications are Collectively Incorporated Herein by Reference for All Purposes

Abidi, P., Zhou, Y., Jiang, J. D. & Liu, L. Extracellular Signal-Regulated Kinase-Dependent Stabilization of Hepatic Low-Density Lipoprotein Receptor mRNA by Herbal Medicine Berberine. Arterioscler Thromb Vasc Biol. 25 (10), 2170-2176 (2005).
Adams C M, Goldstein J L, Brown M S. Cholesterol-induced conformational change in SCAP enhanced by Insig proteins and mimicked by cationic amphiphiles. Proc Natl Acad Sci USA 100:10647-10652 (2003).
Air E L, Strowski M Z, Benoit S C, Conarello S L, Salituro G M, Guan X M, Liu K, Woods S C, Zhang B B. Small molecule insulin mimetics reduce food intake and body weight and prevent development of obesity. Nat. Med. 8(2):179-83 (2002).
American Heart Association; National Heart, Lung, and Blood Institue; Grundy, S. M., Cleeman, J. I., Daniels, S. R., Donato, K. A., Eckel, R H., Franklin, B. A., Gordon, D. J., Krauss, R. M., Savage, P. J., Smith, Jr. S. C., Spertus, J. A. & Costa, F. Diagnosis and management of the metabolic syndrome. An American Heart Association National Heart, Lung, and Blood Institute Scientific Statement. Executive summary. Cardiol Rev. 13(6),322-327 (2005).
Ansell, B. J., Watson, K. E. & Fogelman, A. M. An evidence-based assessment of the NCEP adult treatment panel II guidelines. National cholesterol education program. JAMA 282, 2051-2057 (1999).
Araki, E., Shimada, F., Uzawa, H., Mori, M. & Ebina, Y. Characterization of the promoter region of the human insulin receptor gene. J. Biol. Chem. 262, 16186-16191 (1987).
Aronoff S, Rosenblatt S, Braithwaite S, Egan J W, Mathisen A L, Schneider R L. Pioglitazone hydrochloride monotherapy improves glycemic control in the treatment of patients with type 2 diabetes: a 6-month randomized placebo-controlled dose-response study. The Pioglitazone 001 Study Group. Diabetes Care. 23(11):1605-11 (2000).
Asian-Pacific Region Diabetes Expert Group. Asian-Pacific Region Type 2 Diabetes: Control and treatment. Chinese Journal of Diabetes, 8 (suppl), 60-69 (2000).
Austin M A, Hutter C M, Zimmern R L, Humphries S E. Genetic causes of monogenic heterozygous familial hypercholesterolemia: a HuGE prevalence review. Am J Epidemiol 160:407-420 (2004).
Auwerx J H, Chait A, Wolfbauer G, Deeb S S. Involvement of second messengers in regulation of the low-density lipoprotein receptor gene. Mol Cell Biol 9:2298-2302 (1989).
Bailey C J, Turner R C. Metformin. N Engl J Med. 334(9):574-9 (1996).
Bakker O, Hudig F, Meijssen S, Wiersing W M. Effects of triiodothyronine and amiodarone on the promoter of the human LDL receptor gene. Biochem Biophys Res Commun 249:517-521 (1998).
Balkau, B. & Charles, M. A. Comment on the provisional report from the WHO consultation. European Group for the Study of Insulin Resistance (EGIR). Diabet Med. 16, 442-443 (1999).
Banks G C, Li Y, Reeves R. Differential in vivo modifications of the HMGI(Y) nonhistone chromatin proteins modulate nucleosome and DNA interactions. Biochemistry. 39(28):8333-46 (2000).
Bard-Chapeau E A, Hevener A L, Long S, Zhang E E, Olefsky J M, Feng G S. Deletion of Gab1 in the liver leads to enhanced glucose tolerance and improved hepatic insulin action. Nat. Med. 11(5):567-71 (2005).
Basheeruddin K, Li X, Rechtoris C, Mazzone T. Platelet-derived growth factor enhances Sp1 binding to the LDL receptor gene. Arterioscler Thromb Vasc Biol 15:1248-1254 (1995).
Bays H, Stein E A. Pharmacotherapy for dyslipidaemia—current therapies and future agents. Expert Opin Pharmacother 4:1901-1938 (2003).
Bensch, W. R., Gadski, R. A., Bean, J. S., et al., Effects of LY295427, a low-density lipoprotein (LDL) receptor up-regulator, on LDL receptor gene transcription and cholesterol metabolism in normal and hypercholesterolemic hamsters. J. Pharmacology & Experimental Therapeutics 289, 85-92 (1999).
Briggs M R, Yokoyama C, Wang X, Brown M S, Goldstein J L. Nuclear protein that binds sterol regulatory element of low density lipoprotein receptor promoter. I. Identification of the protein and delineation of its target nucleotide sequence. J Biol Chem 268:14490-14496 (1993).
Brown A J, Sun L, Feramisco J D, Brown M S, Goldstein J L. Cholesterol addition to ER membranes alters conformation of SCAP, the SREBP escort protein that regulates cholesterol metabolism. Mol Cell 10:237-245 (2002).
Brown M S, Goldstein J L. A proteolytic pathway that controls the cholesterol content of membranes, cells, and blood. Proc Natl Acad Sci USA 96:11041-11048 (1999).
Brown M S, Goldstein J L. Receptor-mediated pathway for cholesterol homeostasis. Science 232:34-47 (1986).
Brunetti A, Manfioletti G, Chiefari E, Goldfine I D, Foti D. Transcriptional regulation of human insulin receptor gene by the high-mobility group protein HMGI(Y). FASEB J. 15(2):492-500 (2001).
Brüning J C, Lingohr P, Gillette J, Hanstein B, Avci H, Krone W, Müller-Wieland D, Kotzka J. Estrogen receptor-α and Sp1 interact in the induction of the low density lipoprotein-receptor. J Steroid Biochem Mol Biol 86:113-121 (2003).
Catapano. The low density lipoprotein receptor: structure, function and pharmacological modulation. Pharmac Ther 43:187-219 (1989).
Chang R, Yang E, Chamblis D, Kumar A, Wise J, Mehta K D. In vivo role of the Sp1 site neighboring sterol-responsive element-I in controlling low-density lipoprotein receptor gene expression. Biochem Biophys Res Commun 218:733-739 (1996).
Cho, E. Berberini Hydrochloride, In: Pharmacopoeia of the People's Republic of China. Pharmacopoeia of the People's Republic of China 2, 437-439 (1990).
Cho, E. Berberini Hydrochloride, In: Pharmacopoeia of the People's Republic of China. Pharmacopoeia of the People's Republic of China 2, 437-439 (1990).
Corsini A, Bellosta S, Baetta R, Fumagalli R, Paoletti R, Bernini F. New insights into the pharmacodynamic and pharmacokinetic properties of statins. Pharmacol Ther 84:413-428 (1999).
Davis C G, Van Driel I R, Russell D W, Brown M S, Goldstein J L. The LDL-receptor: identification of aminoacids in cytoplasmic domain required for rapid endocytosis. J Biol Chem 262:4075-4079 (1987).
Defesche J C. Low-density lipoprotein receptor—its structure, function, and mutations. Semin Vasc Med 4:5-11 (2004).
DeFronzo, R A. & Ferrannini, E. Insulin resistance. A multifaceted syndrome responsible for NIDDM, obesity, hypertension, dyslipidemia, and atherosclerotic cardiovascular disease. Diabetes Care 14, 173-194 (1991).
Dixon, D. A., Kaplan, C.D., McIntyre, T. M., Zimmerman, G. A., Prescott, S. M. Post-transcriptional control of cyclooxygenase-2 gene expression. J. Biol. Chem. 275, 11750-11757 (2000).
Dresner, A, Laurent, D., Marcucci, M., Griffin, M. B., Dufour, S., Cline, G. W., Slezak, L. A, Andersen, D. K, Hundal, R. S., Rothman, D. L., Petersen, K F. & Shulman, G. L Effects of free fatty acids on glucose transport and IRS-1-associated phosphatidylinositol 3-kinase activity. J Clin Invest. 103 (2), 253-259 (1999).
Duncan E A, Brown M S, Goldstein J L, Sakai J. Cleavage site for sterol-regulated protease located to a Leu-Ser bond in the lumenal loop of sterol regulatory element-binding protein-2. J Biol Chem 272:12778-12785 (1997).
Duncan E A, Dave U P, Sakai J, Goldstein J L, Brown M S. Second-site cleavage in sterol regulatory element-binding protein occurs at transmembrane junction as determined by cysteine panning. J Biol Chem 273:17801-17809 (1998).
Duntas L H. Thyroid disease and lipids. Thyroid 12:287-293 (2002).
Duvillard L, Florentin E, Lizard G, Petit J M, Galland F, Monier S, Gambert P, Verges B. Cell surface expression of LDL receptor is decreased in type 2 diabetic patients and is normalized by insulin therapy. Diabetes Care 26:1540-1544 (2003).
Eckel, R. H., Grundy, S. M. & Zimmet, p.z. The metabolic syndrome. Lancet 365 (9468), 1415-28 (2005).
Eckel, R H. Lipoprotein lipase. A multifunctional enzyme relevant to common metabolic diseases. N Engl J Med 320, 1060-1068 (1989).
Espenshade P J, Li W P, Yabe D. Sterols block binding of COPII proteins to SCAP, thereby controlling SCAP sorting in ER. Proc Natl Acad Sci USA 99:11694-11699 (2002).
Evans, R. M., Barish, G. D. & Wang, Y.x. PPARs and the complex journey to obesity. Nat. Med. 10(4),355-361 (2004).
Executive Summary of The Third Report of The National Cholesterol Education Program (NCEP) Expert Panel on Detection, Evaluation, And Treatment of High Blood Cholesterol In Adults (Adult Treatment Panel III). JAMA 285, 2486-2497 (2001).
Expert panel on detection evaluation and treatment of high blood cholesterol in adults. Executive summary of the third report of the national cholesterol education program (NCEP). JAMA 285, 2486-2497 (2001).
Farese R V. Function and dysfunction of aPKC isoforms for glucose transport in insulin-sensitive and insulin-resistant states. Am J Physiol Endocrinol Metab. 283(1):E1-11 (2002).
Ferrannini E. Insulin resistance versus insulin deficiency in non-insulin-dependent diabetes mellitus: problems and prospects. Endocr Rev. 19(4):477-90 (1998).
Foti D, Chiefari E, Fedele M, Iuliano R, Brunetti L, Paonessa F, Manfioletti G, Barbetti F, Brunetti A, Croce C M, Fusco A, Brunetti A. Lack of the architectural factor HMGA1 causes insulin resistance and diabetes in humans and mice. Nat. Med. 11(7):765-73 (2005).
Foti D, Iuliano R, Chiefari E, Brunetti A. A nucleoprotein complex containing Sp1, C/EBP beta, and HMGI-Y controls human insulin receptor gene transcription. Mol Cell Biol. 23(8):2720-32 (2003).
Gandino, L., Di Renzo, M. F., Giordano, S., Bussolino, F. & Comoglio, P.M. Protein kinase-c activation inhibits tyrosine phosphorylation of the c-met protein. Oncogene 5 (5), 721-725 (1990).
Gierens H, Nauck M, Roth M, Schinker R, Schürmann C, Schamagl H, Neuhaus G, Wieland H, März W. Interleukin-6 stimulates LDL receptor gene expression via activation of sterol-responsive and Sp1 binding elements. Arterioscler Thromb Vasc Biol 20:1777-1783 (2000).
Gitlin N, Julie N L, Spurr C L, Lim K N, Juarbe H M. Two cases of severe clinical and histologic hepatotoxicity associated with troglitazone. Ann Intern Med. 129(1):36-8 (1998).
Goldstein, J. L. & Brown, M. S. Regulation of the mevalonate pathway. Nature 343, 425-430 (1990).
Goldstein, J. L., Rawson, R. B. & Brown, M. S. Mutant mammalian cells as tools to delineate the sterol regulatory element-binding protein pathway for feedback regulation of lipid synthesis. Archives of Biochemistry and Biophysics 397, 139-148 (2002).
Goto D, Okimoto T, Ono M, Shimotsu H, Abe K, Tsujita Y, Kuwano M (1997) Upregulation of low density lipoprotein receptor by gemfibrozil, a hypolipidemic agent, in human hepatoma cells through stabilization of mRNA transcripts. Arterioscler Thromb Vasc Biol 17:2707-2712.
Graham A, Russell L J. Stimulation of low-density lipoprotein uptake in HepG2 cells by epidermal growth factor via a tyrosine kinase-dependent, but protein kinase C-independent mechanism. Biochem J 298:579-584 (1994).
Grand-Perret, T., Bouillot, A., Perrot, A., Commans, S., Walker, M. & Issandou, M. SCAP ligands are potent new lipid-lowering drugs. Nature Medicine 7, 1332-1338 (2001).
Grundy, S. M. Drug therapy of the metabolic syndrome: minimizing the emerging crisis in polypharmacy. Nat Rev Drug Discov. 5 (4),295-309 (2006).
Grundy, S. M. Statin trials and goals of cholesterol-lowering therapy. Circulation 97, 1436-1439 (1998).
Grundy, S. M., Cleeman, J. I., Daniels, S. R, Donato, K. A, Eckel, R H., Franklin, B. A., Gordon, D. J., Krauss, R M., Savage, P. I, Smith, S. C. Jr, Spertus, J. A, Costa, F; American Heart Association; National Heart, Lung, and Blood Institute. Diagnosis and management of the metabolic syndrome; an American Heart Association/National Heart, Lung, and Blood Institute Scientific Statement. Circulation 112 (17), 2735-2752 (2005).
Guan Y, Hao C, Cha D R, Rao R, Lu W, Kohan D E, Magnuson M A, Redha R, Zhang Y, Breyer M D. Thiazolidinediones expand body fluid volume through PPARgamma stimulation of ENaC-mediated renal salt absorption. Nat Med. 11(8):861-6 (2005).
Haffner S M, D'Agostino R Jr, Mykkanen L, Tracy R, Howard B, Rewers M, Selby J, Savage P J, Saad M F. Insulin sensitivity in subjects with type 2 diabetes. Relationship to cardiovascular risk factors: the Insulin Resistance Atherosclerosis Study. Diabetes Care. 22(4):562-8 (1999).
Hanson, R. L., Pratley, R. E., Bogardus, C., Narayan, K. M., Roumain, J. M., Imperatore, G., Fagot-Campagna, A., Pettitt, D J., Bennett, P. H. & Knowler, W. C. Evaluation of simple indices of insulin sensitivity and insulin secretion for use in epidemiologic studies. Am J Epidemiol. 15 1(2), 190-198 (2000).
Hardardottir I, Grunfeld C, Feingold K R. Effects of endotoxin and cytokines on lipid metabolism. Curr Opin Lipidol 5:207-215 (1994).
Hirano Y, Yoshida M, Shimizu M, Sato R. Direct demonstration of rapid degradation of nuclear sterol regulatory element-binding proteins by the ubiquitin-proteasome pathway. J Biol Chem 276:36431-36437 (2001).
Hobbs H H, Russell D W, Brown M S, Goldstein J L. The LDL receptor locus in familial hypercholesterolemia: mutation analysis of a membrane protein. Annu Rev Genet 24:133-170 (1990).
Hodgin J B, Maeda N. Minireview: estrogen and mouse models of atherosclerosis. Endocrinology 143:4495-4501 (2002).
Horton J D, Goldstein J L, Brown M S. SREBPs: activators of the complete program of cholesterol and fatty acid synthesis in the liver. J Clin Invest 109:1125-1131 (2002).
Horton, J. D., Cuthbert, J. A. & Spady, D. K. Dietary fatty-acids regulate hepatic low-density lipoprotein (LDL) transport by altering LDL receptor protein and messenger-RNA levels. J. Clinc. Invest. 92, 743-749 (1993).
Hsu H Y, Nicholson A C, Hajjar D P. Basic fibroblast growth factor-induced low density lipoprotein receptor transcription and surface expression. Signal transduction pathways mediated by the bFGF receptor tyrosine kinase. J Biol Chem 269:9213-9220 (1994).
Hua X, Nohturfft A, Goldstein J L, Brown M S. Sterol resistance in CHO cells traced to point mutations in SREBP cleavage activating protein (SCAP). Cell 87:415-426 (1996).
Hua X, Sakai J, Ho Y K, Goldstein J L, Brown M S. Hairpin orientation of sterol regulatory element-binding protein-2 in cell membranes as determined by protease protection. J Biol Chem 270:29422-29427 (1995).
Huang W, Mishra V, Batra S, Dillon I, Mehta K D. Phorbol ester promotes histone H3-Ser10 phosphorylation at the LDL receptor promoter in a protein kinase C-dependent manner. J Lipid Res 45:1519-1527 (2004).
Huang, T. H., Kota, B. P., Razmovski, V. & Roufogalis, B. D. Herbal or natural medicines as modulators of peroxisome proliferator-activated receptors and related nuclear receptors for therapy of metabolic syndrome. Basic Clin Pharmacol Toxicol. 96(1),3-14 (2005).
Inukai T, Takanashi K, Takebayashi K, Tayama K, Aso Y, Takiguchi Y, Takemura Y. Estrogen markedly increases LDL-receptor activity in hypercholesterolemic patients. J Med 31:247-261 (2000).
Jensen, M. D., Caruso, M., Heiling, V. & Miles, I M. Insulin regulation of lipolysis in nondiabetic and IDDM subjects. Diabetes 38, 1595-1601 (1989).
Kapoor G S, Atkins B A, Mehta K D. Activation of Raf-1/MEK-1/2/p42/44^{MAPK} cascade alone is sufficient to uncouple LDL receptor expression from cell growth. Mol Cell Biochem 236:13-22 (2002).
Kapoor G S, Golden C, Atkins B, Mehta K D. pp90^{RSK}- and protein kinase C-dependent pathway regulates p42/44^{MAPK}-induced LDL receptor transcription in HepG2 cells. J Lipid Res 44:584-593 (2003).
Kersten S, Desvergne B, Wahli W. Roles of PPARs in health and disease. Nature. 405(6785):421-4 (2000).
Kido Y, Nakae J, Accili D. The Insulin Receptor and Its Cellular Targets. J. Clin Endocrinol Metab Vol. 86, No. 3, 972-979 (2001).
Kim, I K, Fillmore, J. I., Chen, Y., Yu, C., Moore, I. K, Pypaert, M., Lutz, E. P., Kako, Y., Knouff C, Malloy S, Wilder J, Altenburg M K, Maeda N. Doubling expression of the low density lipoprotein receptor by truncation of the 3′-untranslated region sequence ameliorates type III hyperlipoproteinemia in mice expressing the human ApoE2 isoform. J Biol Chem 276:3856-3862 (2001).
Kong W, Abidi P, Kraemer F B, Jiang J D, Liu J. In vivo activities of cytokine oncostatin M in regulation of plasma lipid levels. J Lipid Res 46:1163-1171 (2005).
Kong W, Wei J, Abidi P, Lin M, Inaba S, Li C, Wang Y, Wang Z, Si S, Pan H, Wang S, Wu J, Wang Y, Li Z, Liu J, Jiang JD. Berberine is a novel cholesterol-lowering drug working through a unique mechanism distinct from statins. Nat Med 10:1344-1351 (2004).
Kong, W. J., Liu, J. W. & Jiang, J. D. Human Low-Density Lipoprotein Receptor gene and its regulation. J Mol Med. 84, 29-36 (2006).
Kong, W. J., Wei, J., Abidi, P., Lin, M., Inaba, S., Li, c., Wang, Y. L., Wang, Z., Si, S., Pan, H., Wang, S., Wu, J. D., Wang, Y., Li, Z. R, Liu, J. & Jiang, J. D. Berberine is a novel cholesterol-lowering drug working through a unique mechanism distinct from statins. Nat. Med. 10 (12), 1344-1351 (2004).
Kotzka J, Lehr S, Roth G, Avci H, Knebel B, Müller-Wieland D. Insulin-activated Erk-mitogen-activated protein kinases phosphorylate sterol regulatory element-binding protein-2 at serine residues 432 and 455 in vivo. J Biol Chem 279:22404-22411 (2004).
Kotzka J, Müller-Wieland D, Koponen A, Njamen D, Kremer L, Roth G, Munck M, Knebel B, Krone W. ADD1/SREBP-1c mediates insulin-induced gene expression linked to the MAP kinase pathway. Biochem Biophys Res Commun 249:375-379 (1998).
Kotzka J, Müller-Wieland D, Roth G, Kremer L, Munck M, Schürmann S, Knebel B, Krone W. Sterol regulatory element binding proteins (SREBP)-1a and SREBP-2 are linked to the MAP-kinase cascade. J Lipid Res 41:99-108 (2000).
Kumar A, Middleton A, Chambers T C, Mehta K D. Differential roles of extracellular signal-regulated kinase-1/2 and p38^{MAPK} in interleukin-1β- and tumor necrosis factor-α-induced low density lipoprotein receptor expression in HepG2 cells. J Biol Chem 273:15742-15748 (1998).
LaRosa, J. C. & He, J. V. S. Effect of statins on risk of coronary disease: a meta-analysis of randomized controlled trials. JAMA 282, 2340-2346 (1999).
Lau C W, Yao X Q, Chen Z Y, Ko W H, Huang Y. Cardiovascular actions of berberine. Cardiovasc Drug Rev. 19(3):234-44 (2001).
Lau, C. W., Yao, X. Q., Chen, Z. Y., Ko, W. H., Huang, Y. Cardiovascular actions of berberine. Cardiovasc Drug Rev. 19, 234-244 (2001).
Li C, Briggs M R, Ahlborn T E, Kraemer F B, Liu J. Requirement of Sp1 and estrogen receptor α interaction in 17β-estradiol-mediated transcriptional activation of the low density lipoprotein receptor gene expression. Endocrinology 142:1546-1553 (2001).
Li C, Kraemer F B, Ahlborn T E, Liu J. Induction of low density lipoprotein receptor (LDLR) transcription by oncostatin M is mediated by the extracellular signal-regulated kinase signaling pathway and the repeat 3 element of the LDLR promoter. J Biol Chem 274:6747-6753 (1999).
Lindgren V, Luskey K L, Russell D W, Francke U. Human genes involved in cholesterol metabolism: chromosomal mapping of the loci for the low-density lipoprotein receptor and 3-hydroxy-3-methylglutaryl-coenzyme A reductase with cDNA probe. Proc Natl Acad Sci USA 82:8567-8571 (1985).
Liu J, Ahlborn T E, Briggs M R, Kraemer F B. Identification of a novel sterol-independent regulatory element in the human low density lipoprotein receptor promoter. J Biol Chem 275:5214-5221 (2000).
Liu J, Streiff R, Zhang Y L, Vestal R E, Spence M J, Briggs M R. Novel mechanism of transcriptional activation of hepatic LDL receptor by oncostatin M. J Lipid Res 38:2035-2048 (1997).
Liu, J., Hadjokas, N., Mosley, B., Vestal, R. E. Oncostatin M-specific receptor expression and function in regulating cell proliferation of normal and malignant mammary epithelial cells. Cytokine 10, 295-302 (1998).
Liu, J., Zhang, F., Li, C., Lin, M. and Briggs, M. R. Synergistic activation of human LDL receptor expression by SCAP ligand and cytokine oncostatin M. Arterioscler. Throm. Vasc. Biol 23:90-96, (2003).
Liu. Berberine. Altern Med Rev. 5, 175-177 (2000).
Luo, L. J. Experience of berberine in the treatment of diarrhea. Chin. J. Med. 41, 452-455 (1955).
Luo, L. J. Experience of berberine in the treatment of diarrhea. Chin. J. Med. 41, 452-455 (1955).
Manley S. Haemoglobin A1c—a marker for complications of type 2 diabetes: the experience from the UK Prospective Diabetes Study (UKPDS). Clin Chem Lab Med. 41(9):1182-90 (2003).
Mazzone T, Basheeruddin K, Ping L, Frazer S, Getz G S. Mechanism of the growth-related activation of the low density lipoprotein receptor pathway. J Biol Chem 264:1787-1792 (1989).
Mehta K D. Role of mitogen-activated protein kinases and protein kinase C in regulating low-density lipoprotein receptor expression. Gene Expr 10:153-164 (2002).
Michael M D, Kulkami R N, Postic C, Previs S F, Shulman G I, Magnuson M A, Kahn C R. Loss of insulin signaling in hepatocytes leads to severe insulin resistance and progressive hepatic dysfunction. Mol. Cell. 6(1):87-97 (2000).
Mitchell, P. J. & Tjian, R. Transcriptional regulation in mammalian cells by sequence-specific DNA binding proteins. Science 245, 371-378 (1989).
MRC/BHF. Heart protection study of cholesterol lowering with simvastatin in 20536 high-risk individuals: a randomised placebo-controlled trial. Lancet 360, 7-22 (2002).
Nakahara M, Fujii H, Maloney P R, Shimizu M, Sato R. Bile acids enhance low density lipoprotein receptor gene expression via a MAPK cascade-mediated stabilization of mRNA. J Biol Chem 277:37229-37234 (2002).
Nakamaru, K., Matsumoto, K., Taguchi, T., Suefuji, M., Murata, Y, Igata, M., Kawashima, J., Kondo, T., Motoshima, H., Tsunlzoe, K., Miyamura, N., Toyonaga, T., Araki, E. AICAR, an activator of AMP-activated protein kinase, down-regulates the insulin receptor expression in HepG2 cells. Biochem Biophys Res Commun. 328 (2), 449-454 (2005).
Ness G C, Lopez D. Transcriptional regulation of rat hepatic low-density lipoprotein receptor and cholesterol 7-α-hydroxylase by thyroid hormone. Arch Biochem Biophys 323:404-408 (1995).
Ness G C. Thyroid hormone. Basis for its hypocholesterolemic effect. J Fla Med Assoc 78:383-385 (1991).
Nesto R W, Bell D, Bonow R O, Fonseca V, Grundy S M, Horton E S, Le Winter M, Porte D, Semenkovich C F, Smith S, Young L H, Kahn R; American Heart Association; American Diabetes Association. Thiazolidinedione use, fluid retention, and congestive heart failure: a consensus statement from the American Heart Association and American Diabetes Association. Oct. 7, 2003. Circulation. 108(23):2941-8 (2003).
Ni Y X. [Therapeutic effect of berberine on 60 patients with type II diabetes mellitus and experimental research] Zhong Xi Yi Jie He Za Zhi. 8(12):711-3, 707 (1988).
Nohturfft A, Brown M S, Goldstein J L. Topology of SREBP cleavage activating protein, a polytopic membrane protein with a sterol sensing domain. J Biol Chem 273:17243-17250 (1998).
Nohturfft A, DeBose-Boyd R A, Scheek S, Goldstein J L, Brown M S. Sterols regulate cycling of SREBP cleavage-activating protein (SCAP) between endoplasmic reticulum and Golgi. Proc Natl Acad Sci USA 96:11235-11240 (1999).
Norturfft, A., DeBose-Boyd, R. A., Scheek, S., Goldstein, J. L. & Brown, M. S. Sterols regulate cycling of SREBP cleavage-activating protein (SCAP) between endoplasmic reticulum and Golgi. Proc.Natl.Acad.Sci. USA 96, 11235-11240 (1999).
Ohtani, Hiromasa et al. Effects of dietary cholesterol and fatty acids on plasma cholesterol level and hepatic lipoprotein metabolism. J. of Lipid Res 31 (8): 1413. (1990)
Otto C, Lehrke M, Goke B. Novel insulin sensitizers: pharmacogenomic aspects. Pharmacogenomics. 3(1):99-116 (2002).
Parini P, Angelin B, Rudling M. Importance of estrogen receptors in hepatic LDL receptor regulation. Arterioscler Thromb Vasc Biol 17:1800-1805 (1997).
Pascoe, W. S. & Storlien, L. H. Inducement by fat feeding of basal hyperglycemia in rats with abnormal beta-cell function. Model for study of etiology and pathogenesis of NIDDM. Diabetes. 39 (2), 226-233 (1990).
Phillips L S, Grunberger G, Miller E, Patwardhan R, Rappaport E B, Salzman A; Rosiglitazone Clinical Trials Study Group. Once- and twice-daily dosing with rosiglitazone improves glycemic control in patients with type 2 diabetes. Diabetes Care 24(2):308-15 (2001).
Radhakrishnan A, Sun L P, Kwon H J, Brown M S, Goldstein J L. Direct binding of cholesterol to the purified membrane region of SCAP: mechanism for a sterol-sensing domain. Mol Cell 15:259-268 (2004).
Rawson R B. The SREBP pathway—insights from Insigs and insects. Nat Rev Mol Cell Biol 4:631-640 (2003).
Reaven, G. M. Banting lecture 1988. Role of insulin resistance in human disease. Diabetes 37, 1595-1607 (1988).
Reed, M J., Meszaros, K., Entes, L. J., Claypool, M. D., Pinkett, I G., Gadbois, T. M. & Reaven, G. M. A new rat model of type 2 diabetes: the fat-fed, streptozotocin-treated rat. Metabolism 49 (11), 1390-1394 (2000).
Robinson M J, Cobb M H. Mitogen-activated protein kinase pathways. Curr Opin Cell Biol 9:180-186 (1997).
Rollason, V. & Vogt, N. Reduction of polypharmacy in the elderly: systematic review of the role of pharmacist. Drugs Aging 20, 817-832 (2003).
Roth G, Kotzka J, Kremer L, Lehr S, Lohaus, Meyer H E, Krone W, Müller-Wieland D. MAP kinases Erk1/2 phosphorylate sterol regulatory element-binding protein (SREBP)-1a at serine 117 in vitro. J Biol Chem 275:33302-33307 (2000).
Sakai, J. & Rawson, R. B. The sterol regulatory element-protein pathway: control of lipid homeostasis through regulated intracellular transport. Curr. Opin Lipidol 12, 261-266 (2001).
Saltiel A R, Kahn C R. Insulin signalling and the regulation of glucose and lipid metabolism. Nature. December 13; 414(6865):799-806 (2001).
Saltiel, A. R. New Perspectives into the Molecular Pathogenesis and Treatment of Type 2 Diabetes. Cell, Volume 104, Issue 4, Pages 517-529 (2001).
Seino S, Seino M, Nishi S, Bell G I. Structure of the human insulin receptor gene and characterization of its promoter. Proc Natl Acad Sci USA. 86(1):114-8 (1989).
Singh R P, Dhawan P, Golden C, Kapoor G S, Mehta K D. One-way cross-talk between p38^{MAPK} and p42/44^{MAPK}. Inhibition of p38^{MAPK} induces low density lipoprotein receptor expression through activation of p42/44^{MAPK} cascade. J Biol Chem 274:19593-19600 (1999).
Smith J R, Osborne T F, Goldstein J L, Brown M S. Identification of nucleotides responsible for enhancer activity of sterol regulatory element in low density lipoprotein receptor gene. J Biol Chem 265:2306-2310 (1990).
Soutar A K, Knight B L. Structure and regulation of the LDL-receptor and its gene. Br Med Bull 46:891-916 (1990).
Soutar A K. Intracellular transport of the low-density lipoprotein receptor. Biochem Soc Trans 24:547-552 (1996).
Spady D K. Hepatic clearance of plasma low-density lipoproteins. Semin Liver Dis 12:373-385 (1992).
Srivastava A K, Mehdi M Z. Insulino-mimetic and anti-diabetic effects of vanadium compounds. Diabet Med. 22(1):2-13 (2004).
Steinberger J, Daniels SR. Obesity, insulin resistance, diabetes, and cardiovascular risk in children: an American Heart Association scientific statement from the Atherosclerosis, Hypertension, and Obesity in the Young Committee (Council on Cardiovascular Disease in the Young) and the Diabetes Committee (Council on Nutrition, Physical Activity, and Metabolism). Circulation.; 107: 1448-1453 (2003)
Steinberger J, Moorehead C, Katch V, et al. Relationship between insulin resistance and abnormal lipid profile in obese adolescents. J Pediatr. 126(5 pt 1): 690-695 (1995).
Stopeck A T, Nicholson A C, Mancini F P, Hajjar D P. Cytokine regulation of low density lipoprotein receptor gene transcription in HepG2 cells. J Biol Chem 268:17489-17494 (1993).
Streicher R, Kotzka J, Müller-Wieland D, Siemeister G, Munck M, Avci H, Krone W. SREBP-1 mediates activation of the low density lipoprotein receptor promoter by insulin and insulin-like growth factor-1. J Biol Chem 271:7128-7133 (1996).
Stumvoll M, Goldstein B J, van Haeften T W. Type 2 diabetes: principles of pathogenesis and therapy. Lancet. 365(9467): 1333-46 (2005).
Südhoff T C, Goldstein J L, Brown M S, Russell D W. The LDL-receptor gene. A mosaic of exons shared with different proteins. Science 228:815-822 (1985).
Südhoff T C, Van Der Westhuyzen D R, Goldstein J L, Brown M S, Russell D W. Three direct repeats and a TATA-like sequence are required for regulated expression of the human low density lipoprotein receptor gene. J Biol Chem 262:10773-10779 (1987).
Sugiura, R., Kita, A., Shimizu, Y., Shuntoh, H., Sio, S. O. & Kuno, T. Feedback regulation of MAPK signaling by an RNA-binding protein. Nature 424, 961-965 (2003).
Taylor C E, Greenough W B 3rd. Control of diarrheal diseases. Annu Rev Public Health. 10:221-44 (1989).
Taylor S I, Accili D, Imai Y. Insulin resistance or insulin deficiency. Which is the primary cause of NIDDM? Diabetes. 43(6):735-40 (1994).
Tewari D S, Cook D M, Taub R. Characterization of the promoter region and 3′ end of the human insulin receptor gene. J Biol Chem. 264(27):16238-45 (1989).
Tolleshaug H, Goldstein J L, Schneider W J, Brown M S. Posttranslational processing of the LDL receptor and its genetic disruption in familial hypercholesterolemia. Cell 30:715-724 (1982).
Ugawa, T., Kakuta, H., Moritani, H. and Inagaki, O. Effect of YM-53601, a novel squalene synthase inhibitor, on the clearance rate of plasma LDL and VLDL in hamsters. British J. of Pharmacology 137, 561-567 (2002).
Vasudevan A R, Balasubramanyam A. Thiazolidinediones: a review of their mechanisms of insulin sensitization, therapeutic potential, clinical efficacy, and tolerability. Diabetes Technol Ther. 6(6):850-63 (2004).
Wade D P, Knight B L, Soutar A K. Regulation of low-density-lipoprotein-receptor mRNA by insulin in human hepatoma HepG2 cells. Eur J Biochem 181:727-731 (1989).
Wang X, Briggs M R, Hua X, Yokoyama C, Goldstein J L, Brown M S Nuclear protein that binds sterol regulatory element of low density lipoprotein receptor promoter. 11. Purification and characterization. J Biol Chem 268:14497-14504 (1993).
Wang X, Sato R, Brown M S, Hua X, Goldstein J L (1994) SREBP-1, a membrane-bound transcription factor released by sterol-regulated proteolysis. Cell 77:53-62 (1989).
Wilson G M, Roberts E A, Deeley R G. Modulation of LDL receptor mRNA stability by phorbol esters in human liver cell culture models. J Lipid Res 38:437-446 (1997).
Wilson G M, Vasa M Z, Deeley R G. Stabilization and cytoskeletal-association of LDL receptor mRNA are mediated by distinct domains in its 3′ untranslated region. J Lipid Res 39:1025-1032 (1998).
Windler E E T, Kovanen P T, Chao Y, Brown M S, Havel R J, Goldstein J L. The estradiol-stimulated lipoprotein receptor of rat liver. J Biol Chem 255:10464-10471 (1980).
Winzell, M. S., & Ahren B. The high-fat-fed mouse. A model for studying mechanisms and treatment of impaired glucose tolerance and type 2 diabetes. Diabetes, 53, s215-s219 (2003).
World Health Organization Expert Committee. Definition, diagnosis and classification of diabetes mellitus and its complications. Report of a WHO consultation, part 1: diagnosis and classification of diabetes mellitus, World Health Organization, Geneva (1999).
Yabe D, Brown M S, Goldstein J L. Insig-2, a second endoplasmic reticulum protein that binds SCAP and blocks export of sterol regulatory element-binding proteins. Proc Natl Acad Sci USA 99:12753-12758 (2002).
Yamamoto R, Kobayashi H, Yanagita T, Yokoo H, Kurose T, Shiraishi S, Minami S, Matsukura S, Wada A. Up-regulation of cell surface insulin receptor by protein kinase C-alpha in adrenal chromaffin cells: involvement of transcriptional and translational events. J Neurochem. 75(2):672-82 (2000).
Yamamoto T, Davis C G, Brown M S, Schneider W J, Casey M L, Goldstein J L, Russell D W. The human LDL receptor: a cysteine-rich protein with multiple Alu sequences in its mRNA. Cell 39:27-38 (1984).
Yang T, Espenshade P J, Wright M E, Yabe D, Gong Y, Aebersold R, Goldstein J L, Brown M S. Crucial step in cholesterol homeostasis: sterols promote binding of SCAP to Insig-1, a membrane protein that facilitates retention of SREBPs in ER. Cell 110:489-500 (2002).
Yang, T., Goldstein, J. L. & Brown, M. S. Overexpression of membrane domain of SCAP prevents sterols from inhibiting SCAP.SREBP exit from endoplasmic reticulum. J. Biol. Chem. 275, 29881-29886 (2000).
Yeung, A. & Tsao, P. Statin therapy: beyond cholesterol lowering and antiinflammatory effects. Circulation 105, 2937-2938 (2002).
Yki-Jarvinen H. Pathogenesis of non-insulin-dependent diabetes mellitus. Lancet. 343(8889):91-5 (1994).
Yokoyama C, Wang X, Briggs M R, Admon A, Wu J, Hua X, Goldstein J L, Brown M S. SREBP-1, a basic-helix-loop-helix-leucine zipper protein that controls transcription of the low density lipoprotein receptor gene. Cell 75:187-197 (1993).
Zeng X, Zeng X. Relationship between the clinical effects of berberine on severe congestive heart failure and its concentration in plasma studied by HPLC. Biomed Chromatogr. 13(7):442-4 (1999).
Zeng X H, Zeng X J, Li Y Y. Efficacy and safety of berberine for congestive heart failure secondary to ischemic or idiopathic dilated cardiomyopathy. Am J Cardiol. 15; 92(2): 173-6 (2003).
Zhang B, Salituro G, Szalkowski D, Li Z, Zhang Y, Royo I, Vilella D, Diez M T, Pelaez F, Ruby C, Kendall R L, Mao X, Griffin P, Calaycay J, Zierath J R, Heck J V, Smith R G, Moller D E. Discovery of a small molecule insulin mimetic with antidiabetic activity in mice. Science. 284(5416):974-7 (1999).
Zhang F, Lin M, Abidi P, Thiel G, Liu J. Specific interaction of Egr1 and c/EBPβ leads to the transcriptional activation of the human low density lipoprotein receptor gene. J Biol Chem 278:44246-44254 (2003).
Zhang, F., Ahlborn, T. E., Li, C., Kraemer, F. B. & Liu, J. Identification of Egr1 as the oncostatin M-induced transcription activator that binds to the sterol-independent regulatory element of the human LDL receptor promoter. J. Lipid Res. 43, 1477-1485 (2002).
Zhao, S. P. Etiology and diagnosis of hyperlipidemia. Clinical Serum and Lipid 1, 72-94 (1997).
Zubiaga A M, Belasco J G, Greenberg M E. The nonamer UUAUUUAUU is the key AU-rich sequence motif that mediates mRNA degradation. Mol Cell Biol 15:2219-2230 (1995).

1-142. (canceled)

143. A method for preventing or treating metabolic syndrome in a mammalian subject comprising administering an anti-metabolic syndrome effective amount of a berberine compound or berberine related or derivative compound of Formula I, or a pharmaceutically-acceptable salt, isomer, enantiomer, solvate, hydrate, polymorph or prodrug thereof, to said subject 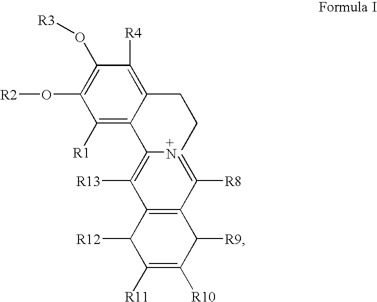
wherein each of R₁, R₂, R₃, R₄, R₈, R₉, R₁₀, R₁₁, R₁₂ and/or R₁₃ is, independently, collectively, or in any combination, selected from hydrogen, halogen, hydroxy, alkyl, alkoxy, nitro, amino, trifluoromethyl, cycloalkyl, (cycloalkyl)alkyl, alkanoyl, alkanoyloxy, aryl, aroyl, aralkyl, nitrile, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, alkanoylamino, carbamoyl, carbamyl, carbonylamino, alkylsulfonylamino, oligosaccharide and heterocyclo groups.

144. The method of claim 143, wherein R₁ is selected from methyl, ethyl, hydroxyl, or methoxy; R₂ is selected from H, methyl, ethyl, methene; R₃ is selected from H, methyl, ethyl, methene; R₄ is selected from methyl, ethyl, hydroxyl, or methoxy; R₈ is selected from straight or branched (C1-C6)alkyl, including substitution selected from methyl, ethyl, n-propyl, 1-methylethyl, n-butyl, 1-methylpropyl, 2-methylpropyl, 1,1-dimethylethyl, n-pentyl, 2-methylbutyl, 1,1-dimethylpropyl, 2,2 dimethylpropyl, 3-methylbutyl, n-hexyl, 1-methylpentyl, 1,1-dimethylbutyl, 2,2-dimethylbutyl, 3-methylpentyl, 1,2-dimethylbutyl, 1,3-dimethyl and 1-methyl-2ethylpropyl; R₉ is selected from methyl, ethyl, hydroxyl, Cl, Br; R₁₀ is selected from methyl, ethyl, hydroxyl, Cl, Br; R₁₁ is selected from methyl, ethyl, hydroxyl, Cl, Br; R₁₂ is selected from methyl, ethyl, hydroxyl, Cl, Br; and R₁₃ is selected from straight or branched (C1-C6)alkyl, including substitution selected from methyl, ethyl, n-propyl, 1-methylethyl, n-butyl, 1-methylpropyl, 2-methylpropyl, 1,1-dimethylethyl, n-pentyl, 2-methylbutyl, 1,1-dimethylpropyl, 2,2 dimethylpropyl, 3-methylbutyl, n-hexyl, 1-methylpentyl, 1,1-dimethylbutyl, 2,2-dimethylbutyl, 3-methylpentyl, 1,2-dimethylbutyl, 1,3-dimethyl and 1-methyl-2ethylpropyl.

145. The method of claim 143, further comprising administering a secondary metabolic syndrome therapeutic agent that is effective in a combinatorial formulation or coordinate treatment regimen with said berberine agent or other adjunctive therapeutic agent that is effective in a combinatorial formulation or coordinate treatment regimen with said berberine compound or berberine related or derivative compound of Formula I to treat or prevent metabolic syndrome or a related symptom or condition thereof in said subject.

146. The method of claim 145, wherein the secondary metabolic syndrome therapeutic or adjunctive therapeutic agent is administered to said subject in a coordinate administration protocol, simultaneously with, prior to, or after, administration of said berberine to the subject.

147. The method of claim 145, wherein the secondary metabolic syndrome therapeutic or adjunctive therapeutic agent is selected from anti-hyperlipidemic agents, anti-dyslipidemic agents, plasma HDL-raising agents, cholesterol-uptake inhibitors, cholesterol biosynthesis inhibitors, HMG-CoA reductase inhibitors, HMG-CoA synthase inhibitors, squalene epoxidase inhibitors, squalene synthetase inhibitors, acyl-coenzyme A cholesterol acyltransferase (ACAT) inhibitors, nicotinic acid and the salts thereof, niacinamide, cholesterol absorption inhibitors, bile acid sequestrant anion exchange resins, LDL receptor inducers, fibrates, vitamin B6, vitamin B12, vitamin B3, anti-oxidant vitamins, angiotensin II receptor (AT₁) antagonist, renin inhibitors, platelet aggregation inhibitors, hormones, insulin, ion exchange resins, omega-3 oils, benfluorex, ethyl icosapentate, amlodipine, insulin sensitizers, protein tyrosine phosphatase-1B (PTP-1B) inhibitors, dipeptidyl peptidase IV (DP-IV) inhibitors, insulin mimetics, sequestrants, nicotinyl alcohol, nicotinic acid, PPARα agonists, PPARγ agonists, PPARα/γ dual agonists, neuropeptide Y5 inhibitors, β₃ adrenergic receptor agonists, ileal bile acid transporter inhibitors, anti-inflammatories, cyclo-oxygenase 2 selective inhibitors, sulfonylureas, DPP-4 blockers, biguanides, alpha-glucosidase inhibitors, D-phenylalanine derivatives, meglitinides, diuretics, beta-blockers, angiotensin-converting enzyme (ACE) inhibitors, calcium channel blockers, vasodilators, angiotensin II receptor blockers, and alpha blockers.

148. The method of claim 147, wherein the secondary metabolic syndrome therapeutic or adjunctive therapeutic agent is a statin or HMG-CoA reductase inhibitor.

149. The method of claim 147, wherein the secondary metabolic syndrome therapeutic or adjunctive therapeutic agent is a cholesterol-uptake inhibitor or a cholesterol biosynthesis inhibitor.

150. The method of claim 147, wherein the secondary metabolic syndrome therapeutic or adjunctive therapeutic agent is an acyl-coenzyme A cholesterol acyltransferase (ACAT) inhibitor.

151. The method of claim 147, wherein the secondary metabolic syndrome therapeutic or adjunctive therapeutic agent is a cholesterol absorption inhibitor.

152. The method of claim 147, wherein the secondary metabolic syndrome therapeutic or adjunctive therapeutic agent is an anion exchange resin.

153. The method of claim 147, wherein the secondary metabolic syndrome therapeutic or adjunctive therapeutic agent is a fibrate.

154. The method of claim 147, wherein the secondary metabolic syndrome therapeutic or adjunctive therapeutic agent is a sulfonylurea.

155. The method of claim 147, wherein the secondary metabolic syndrome therapeutic or adjunctive therapeutic agent is a biguanide.

156. The method of claim 147, wherein the secondary metabolic syndrome therapeutic or adjunctive therapeutic agent is a thiazolidinedione.

157. The method of claim 147, wherein the secondary metabolic syndrome therapeutic or adjunctive therapeutic agent is an alpha-glucosidase inhibitor.

158. The method of claim 147, wherein the secondary metabolic syndrome therapeutic or adjunctive therapeutic agent is a diuretic.

159. The method of claim 147, wherein the secondary metabolic syndrome therapeutic or adjunctive therapeutic agent is a beta-blocker.

160. The method of claim 147, wherein the secondary metabolic syndrome therapeutic or adjunctive therapeutic agent is an ACE inhibitor.

161. The method of claim 147, wherein the secondary metabolic syndrome therapeutic or adjunctive therapeutic agent is a calcium channel blocker.

162. The method of claim 147, wherein the secondary metabolic syndrome therapeutic or adjunctive therapeutic agent is a vasodilator.

163. The method of claim 147, wherein the secondary metabolic syndrome therapeutic or adjunctive agent is an angiotensin II receptor blocker.

164. The method of claim 147, wherein the secondary metabolic syndrome therapeutic or adjunctive agent is an alpha blocker.

165. The method of claim 147, wherein the secondary metabolic syndrome therapeutic or adjunctive agent is an alpha 2 agonist.

166. The method of claim 143, further comprising advising or engaging the subject to undertake an additional therapeutic treatment selected from the group consisting of exercise, diet modification, or surgery.

167. The method of claim 143, wherein said anti-metabolic syndrome treating effective amount comprises between about 10 to about 1500 mg of said berberine compound or berberine related or derivative compound of Formula I per day.

168. The method of claim 143, wherein said anti-metabolic syndrome treating effective amount comprises between about 20 mg to about 1000 mg of said berberine compound or berberine related or derivative compound of Formula I per day.

169. The method of claim 143, wherein said anti-metabolic syndrome effective amount comprises between about 25 mg to about 750 mg of said berberine compound or berberine related or derivative compound of Formula I per day.

170. The method of claim 143, wherein said anti-metabolic syndrome effective amount comprises between about 50 mg to about 500 mg of berberine per day.

171. The method of claim 143, wherein said anti-metabolic syndrome effective amount of said berberine compound or berberine related or derivative compound of Formula I is administered one, two, three, or four times per day.

172. The method of claim 143, wherein the administration of said berberine compound or berberine related or derivative compound of Formula I is effective to decrease body weight by about 1-25%.

173. The method of claim 143, wherein the administration of said berberine compound or berberine related or derivative compound of Formula I is effective to decrease body weight by about 3-15%.

174. The method of claim 143, wherein the administration of said berberine compound or berberine related or derivative compound of Formula I is effective to decrease body fat percentage by about 5-50%.

175. The method of claim 143, wherein the administration of said berberine compound or berberine related or derivative compound of Formula I is effective to decrease body fat percentage by about 15-30%.

176. The method of claim 143, wherein the administration of said effective amount of the berberine compound or berberine related or derivative compound of Formula I is anti-metabolic syndrome effective to decrease total cholesterol in said subject to less than about 200 mg/dL.

177. The method of claim 143, wherein the administration of said effective amount of the berberine compound or berberine related or derivative compound of Formula I is anti-metabolic syndrome effective to decrease total cholesterol in said subject to less than about 175 mg/dL.

178. The method of claim 143, wherein the administration of berberine is anti-metabolic syndrome effective to decrease LDL levels in said subject to less than about 130 mg/dL.

179. The method of claim 143, wherein the administration of said effective amount of the berberine compound or berberine related or derivative compound of Formula I is anti-metabolic syndrome effective to decrease LDL levels in said subject by at least about 20%.

180. The method of claim 143, wherein the administration of said effective amount of the berberine compound or berberine related or derivative compound of Formula I is anti-metabolic syndrome effective to decrease triglycerides in said subject to less than about 150 mg/dL.

181. The method of claim 143, wherein the administration of said effective amount of the berberine compound or berberine related or derivative compound of Formula I is anti-metabolic syndrome effective to decrease triglycerides in said subject by about 20 mg/dL to about 50 mg/dL.

182. The method of claim 143, wherein the administration of said effective amount of the berberine compound or berberine related or derivative compound of Formula I is anti-metabolic syndrome effective to decrease hs-CRP in said subject to about 2.0 mg/L.

183. The method of claim 143, wherein the administration of said effective amount of the berberine compound or berberine related or derivative compound of Formula I is anti-metabolic syndrome effective to decrease hs-CRP in said subject by about 0.5 mg/L to about 2.0 mg/L.

184. The method of claim 143, wherein the administration of said effective amount of the berberine compound or berberine related or derivative compound of Formula I is anti-metabolic syndrome effective to decrease fasting glucose by about 10% to 40%, or to less than about 100-125 mg/dL.

185. The method of claim 143, wherein the administration of said effective amount of the berberine compound or berberine related or derivative compound of Formula I is anti-metabolic syndrome effective to decrease non-fasting blood glucose to between about 140 to 200 mg/dL.

186. The method of claim 143, wherein the administration of said effective amount of the berberine compound or berberine related or derivative compound of Formula I is anti-metabolic syndrome effective to increase glucose consumption in a hyperinsulinemic euglycemic clamp study to above 7.5 mg/min.

187. The method of claim 143, wherein the administration of said effective amount of the berberine compound or berberine related or derivative compound of Formula I is anti-metabolic syndrome effective to decrease glycohemoglobin (HbA1c) to less than 14%.

188. The method of claim 143, wherein the administration of said effective amount of the berberine compound or berberine related or derivative compound of Formula I is anti-metabolic syndrome effective to decrease glycohemoglobin (HbA1c) to between 5 and 8%.

189. The method of claim 143, wherein the administration of said effective amount of the berberine compound or berberine related or derivative compound of Formula I is anti-metabolic syndrome effective to increase ¹³CO₂ consumption by about 15% to about 30%.

190. The method of claim 143, wherein the administration of said effective amount of the berberine compound or berberine related or derivative compound of Formula I is anti-metabolic syndrome effective to lower blood pressure to less than about 150/100 mmHg.

191. The method of claim 143, wherein the administration of said effective amount of the berberine compound or berberine related or derivative compound of Formula I is anti-metabolic syndrome effective to lower a d-dimer level by about 15%.

192-528. (canceled)


SEQUENCE LISTING (3 sequences)

SEQ ID NO: 1 - DNA, 23 bp, Artificial Sequence
  Description of Artificial Sequence Synthetic primer
gctggagagcaactgcaraaggc

SEQ ID NO: 2 - DNA, 21 bp, Artificial Sequence
  Description of Artificial Sequence Synthetic primer
gcagaccagtagatccagagg

SEQ ID NO: 3 - DNA, 10 bp, Artificial Sequence
  Description of Artificial Sequence Synthetic oligonucleotide
atcaccccac